(19) United States
(12) Patent Application Publication
(10) Pub. No.: US 2018/0185516 A1
(43) Pub. Date: Jul. 5, 2018

(54) DELIVERY OF TARGET SPECIFIC NUCLEASES

(71) Applicants: Sangamo Therapeutics, Inc., Richmond, CA (US); Acuitas Therapeutics, Inc., Vancouver (CA)

(72) Inventors: Steven M. Ansell, Vancouver (CA); Christopher Barbosa, Vancouver (CA); Anthony Conway, Richmond, CA (US); Xinyao Du, Vancouver (CA); Michael J. Hope, Vancouver (CA); Michael C. Holmes, Richmond, CA (US); Gary K. Lee, Richmond, CA (US); Paulo Jia Ching Lin, Vancouver (CA); Thomas Madden, Vancouver (CA); Barbara Mui, Vancouver (CA)

(21) Appl. No.: 15/835,957

(22) Filed: Dec. 8, 2017

Related U.S. Application Data
(60) Provisional application No. 62/432,042, filed on Dec. 9, 2016.
(60) Provisional application No. 62/458,373, filed on Feb. 13, 2017.
(60) Provisional application No. 62/503,470, filed on May 9, 2017.
(60) Provisional application No. 62/559,186, filed on Sep. 15, 2017.

(51) Int. Cl. A61K 48/00; C12N 15/11; C12N 9/22; C12N 15/90; A61K 35/76; A61K 9/51

(52) U.S. Cl. CPC A61K 48/0008; C12N 15/11; C12N 9/22; C12N 2310/20; A61K 35/76; A61K 9/5123; C12N 2800/80; C12N 15/907

(57) ABSTRACT

Described herein are lipid nanoparticles comprising cationic lipids and other lipids and also comprising engineered nucleases facilitate transfer of nucleic acids to cells.

### CROSS-REFERENCE TO RELATED APPLICATIONS

The present application claims the benefit of U.S. Provisional Application No. 62/432,042, filed Dec. 9, 2016; U.S. Provisional No. 62/458,373, filed Feb. 13, 2017; U.S. Provisional No. 62/503,470, filed May 9, 2017; and U.S. Provisional No. 62/559,186, filed Sep. 15, 2017, the disclosures of which are hereby incorporated by reference in their entireties.

### TECHNICAL FIELD

The present disclosure is in the fields of polypeptide and genome engineering and the use of cationic lipids and other lipid components to facilitate transfer of nucleic acids to cells.

### BACKGROUND

Gene therapy holds enormous potential for a new era of human therapeutics. These methodologies will allow treatment for conditions that heretofore have not been addressable by standard medical practice. One area that is especially promising is the ability to add a transgene to a cell to cause that cell to express a product that previously was not being produced in that cell. Examples of uses of this technology include the insertion of a gene encoding a therapeutic protein, insertion of a coding sequence encoding a protein that is somehow lacking in the cell or in the individual and insertion of a sequence that encodes a structural nucleic acid such as a microRNA.

Artificial nucleases, such as engineered zinc finger nucleases (ZFN), transcription-activator like effector nucleases (TALENs), the CRISPR/Cas system with an engineered crRNA/tracr RNA (‘single guide RNA’), also referred to as RNA guided nucleases, and/or nucleases based on the Argonaute system (e.g., from T. thermophilus, known as ‘TtAgo’, (Swarts et al (2014) Nature 507(7491): 258-261), comprise DNA binding domains (nucleotide or polypeptide) associated with or operably linked to cleavage domains, and have been used for targeted alteration of genomic sequences. For example, nucleases have been used to insert exogenous sequences, inactivate one or more endogenous genes, create organisms (e.g., crops) and cell lines with altered gene expression patterns, and the like. See, e.g., U.S. Pat. Nos. 9,394,545; 9,150,847; 9,045,763; 9,005,973; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; and 20150056705. For instance, a pair of nucleases (e.g., zinc finger nucleases, TALENs, dCas-Fok fusions) may be used to cleave genomic sequences. Each member of the pair generally includes an engineered (non-naturally occurring) DNA-binding protein linked to one or more cleavage domains (or half-domains) of a nuclease. When the DNA-binding proteins bind to their target sites, the cleavage domains that are linked to those DNA binding proteins are positioned such that dimerization and subsequent cleavage of the genome can occur.

Transgenes can be delivered to a cell by a variety of ways, such that the transgene becomes integrated into the cell's own genome and is maintained there. In recent years, a strategy for transgene integration has been developed that uses cleavage with site-specific nucleases for targeted insertion into a chosen genomic locus (see, e.g., co-owned U.S. Pat. No. 7,888,121). Nucleases, such as zinc finger nucleases (ZFNs), transcription activator-like effector nucleases (TALENs), or nuclease systems such as the CRISPR/Cas system (utilizing an engineered guide RNA), are specific for targeted genes and can be utilized such that the transgene construct is inserted by either homology directed repair (HDR) or by end capture during non-homologous end joining (NHEJ) driven processes. See, e.g., U.S. Pat. Nos. 9,394,545; 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

Transgenes may be introduced and maintained in cells in a variety of ways. Following a “cDNA” approach, a transgene is introduced into a cell such that the transgene is maintained extra-chromosomally rather than via integration into the chromatin of the cell. The transgene may be maintained on a circular vector (e.g. a plasmid, or a non-integrating viral vector such as AAV or Lentivirus), where the vector can include transcriptional regulatory sequences such as promoters, enhancers, polyA signal sequences, introns, and splicing signals (PCT/US2016/42099). An alternate approach involves the insertion of the transgene in a highly expressed safe harbor location such as the albumin gene (see U.S. Pat. Nos. 9,394,545 and 9,150,847). This approach has been termed the In Vivo Protein Replacement Platform™ or IVPRP. Following this approach, the transgene is inserted into the safe harbor (e.g., Albumin) gene via nuclease-mediated targeted insertion where expression of the transgene is driven by the Albumin promoter. The transgene is engineered to comprise a signal sequence to aid in secretion/excretion of the protein encoded by the transgene.

“Safe harbor” loci include loci such as the AAVS1, HPRT, Albumin and CCR5 genes in human cells, and Rosa26 in murine cells. See, e.g., U.S. Pat. Nos. 9,394,545; 9,150,847; 7,888,121; 7,972,854; 7,914,796; 7,951,925; 8,110,379; 8,409,861; 8,586,526; U.S. Patent Publications 20030232410; 20050208489; 20050026157; 20060063231; 20080159996; 201000218264; 20120017290; 20110265198; 20130137104; 20130122591; and 20140017212. Nuclease-mediated integration offers the prospect of improved transgene expression, increased safety and expressional durability, as compared to classic integration approaches that rely on random integration of the transgene, since it allows exact transgene positioning for a minimal risk of gene silencing or activation of nearby oncogenes.

The clinical translation of gene therapy has been hampered by the issues surrounding the delivery of nucleic acids, both in vivo and in vitro/ex vivo. Viral approaches offer great promise but they too have limitations. For example, although vectors such as adeno-associated virus (AAV) are generally considered safe, their payload is limited (<4.8 kb) and the efficiency of certain serotypes is reduced by the presence of innate antibodies in otherwise potential patients. In addition, multiple dosing can be similarly affected by the development of an immune response post the initial dosing. In addition, manufacturing of such viral vectors can be less than straightforward, especially when one considers the eventual viral yields that will be required to use these delivery vectors in the clinic (Nayerossadat et al (2012) Adv BiomedRes 1:27). The ideal delivery vehicle, viral or non-viral should have low antigenic potential, high capacity to accommodate genetic material, high transduction efficiency, controlled and targeted transgene expression and a facile manufacturing processes, all while having reasonable expense and safety for both patients and the environment (Wang et al (2015) J. Funct Biomat 6:379-394).

One non-viral approach relates to the use of nanoparticles comprising nucleic acids to deliver their payloads to target cells. Nanoparticles can be solid in nature, and comprise materials including polysaccharides, lipids, proteins, biodegradable polymers, and metal oxides. Other nanoparticles are in a liquid form, and are mainly liposomes, micelles or emulsion systems composed of amphiphilic molecules or polymers. Lipid nanoparticles (LNP) are one of the most promising types of nanoparticles due to high encapsulating efficiency of nucleic acids, high stability and compatibility with biologic environments (Lee et al (2016) Am J Cancer Res 6(5): 1118-1134).

However, delivery via LNPs is currently inefficient due primarily to suboptimal characteristics of the generally available lipid components.

Thus, there remains a need for additional methods and compositions to deliver gene therapy reagents to biological systems.

### SUMMARY

The present disclosure provides methods and compositions to increase the efficiency of gene therapy through the use of LNP reagents comprising gene therapy reagents. Thus, described herein are compositions comprising LNPs capable of delivering mRNAs encoding engineered transcription factors or engineered nucleases, and/or DNAs encoding engineered transcription factors, engineered nucleases and/or donor (transgenes) for use in gene therapy. The disclosure also provides methods of using these compositions for regulation of a gene of interest, targeted cleavage of cellular chromatin in a region of interest to knock out one or more genes, and/or integration of a transgene via targeted integration at a predetermined region of interest in cells.

Thus, in one aspect, described herein are novel LNPs comprising one or more cationic lipids and comprising one or more nucleic acids (e.g., a nucleic acid (DNA and/or mRNA) encoding one or more proteins such as one or more engineered transcription factors (e.g., activators or repressors), one or more engineered nucleases, one or more donors (transgenes), one or more shRNAs, etc.). Upon delivery to a cell (in vitro or in vivo) the proteins encoded by the nucleic acids exhibit increased activity in the cell and/or provide improved tolerability to the delivery process in the cell, as compared to when the nucleic acid(s) are delivered by other (nonviral or viral) delivery mechanisms. In some embodiments, a nucleic acid of the LNP comprises one or more mRNAs that encode one or more nucleases or transcription factors. In some aspects, the engineered transcription factors comprise one or more zinc finger proteins (ZF-TF), one or more TAL-effector domain proteins (TALEs), one or more CRISPR/Cas transcription factors (CRISPR-TFs). In some aspects, the nuclease(s) comprise(s) one or more zinc finger nucleases (ZFNs). In further aspects, the nuclease(s) comprise(s) a Tale Effector-like nuclease (TALEN), one or more CRISPR/Cas nuclease(s), one or more MegaTALs and/or one or more meganuclease(s). In some embodiments, a nucleic acid of the LNP comprises a donor DNA. In some aspects, the donor DNA is a plasmid, a minigene, or a linear DNA. In further aspects, the nucleic acid comprising a DNA comprising a transgene for delivery to the cell, can serve as template for targeted integration, or can be maintained extra-chromosomally.

In some embodiments, the LNPs comprise cationic lipid molecules. In some aspects, the LNPs also comprise neutral lipids, charged lipids, steroids, including cholesterols and/or their analogs, and/or polymer conjugated lipids.

In some embodiments, the cationic lipids are selected from the molecules having the following Formulas (I, II, III and IV):

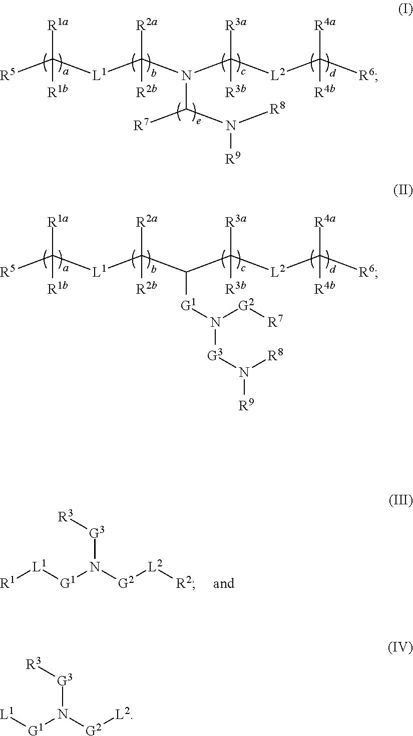

or a pharmaceutically acceptable salt, tautomer or stereoisomer thereof, wherein R¹, R², R³, R^{1a}, R^{1b}, R^{2a}, R^{2b}, R^{3a}, R^{3b}, R^{4a}, R^{4b}, R⁵, R⁶, R⁷, R⁸, R⁹, L¹, L², G¹, G², G³, a, b, c, d and e are as defined herein for each of Formulas (I), (II), (III) and (IV). In some embodiments, the cationic lipid is I-5 or I-6, as shown below:

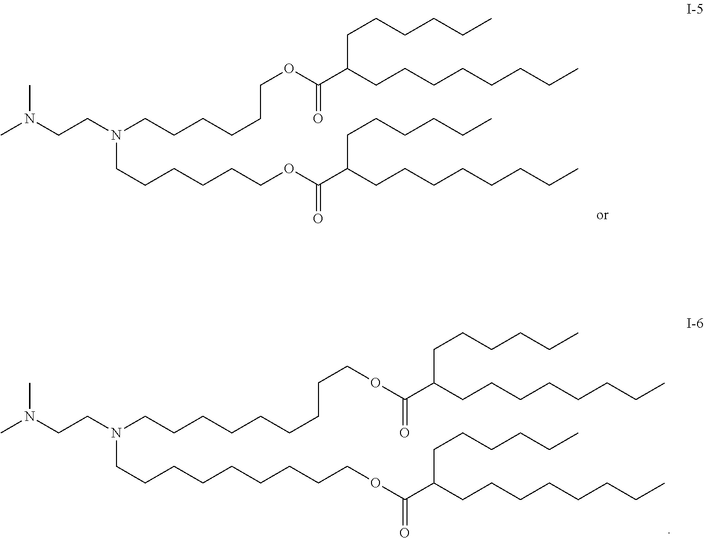

In other embodiments, the cationic lipid is II-9, II-11 or II-36, as shown below:

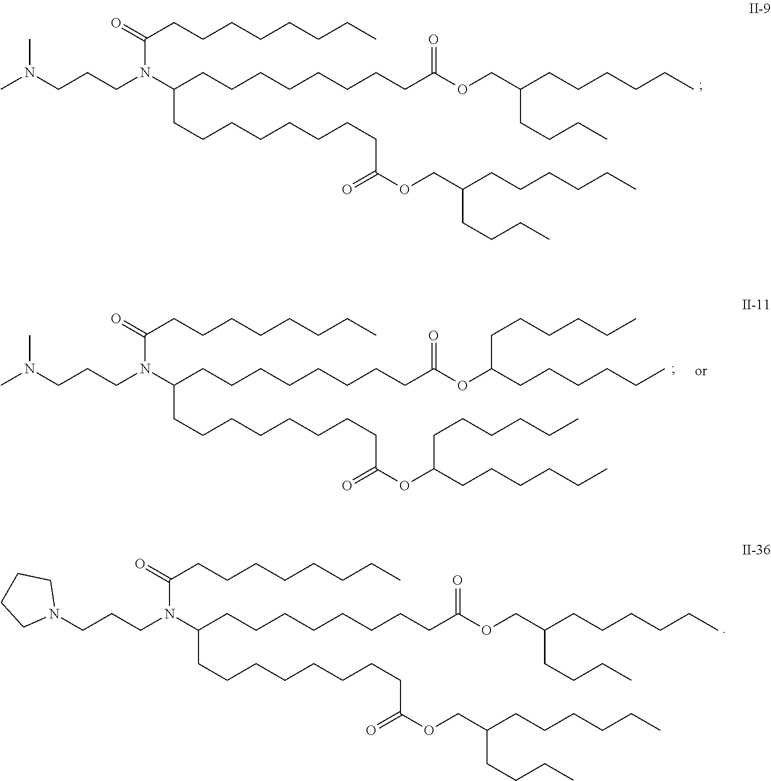

In other embodiments, the cationic lipid is 111-25, III-45 or III-49 as shown below:

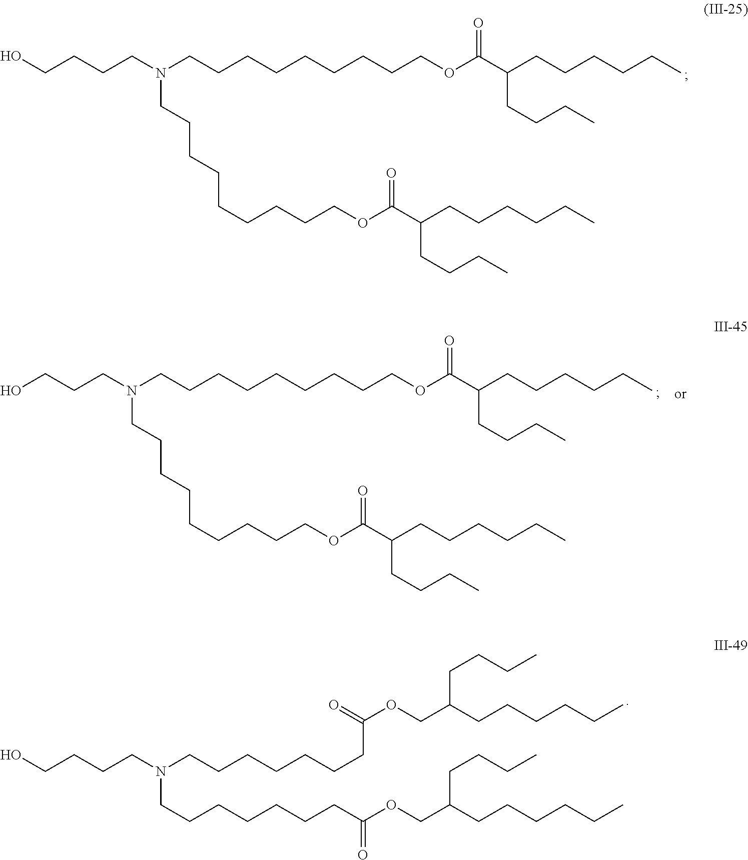

In other different embodiments, the cationic lipid is IV-12 as shown below:

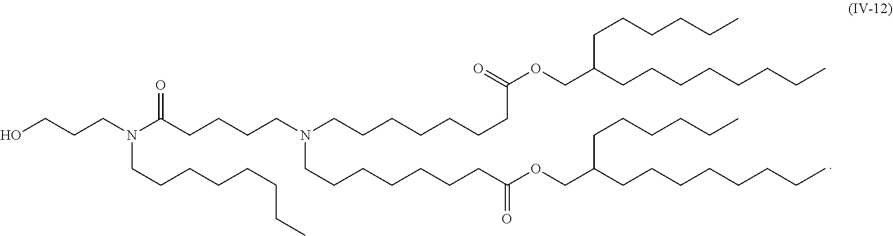

Pharmaceutical compositions comprising one or more LNPs as described herein are also provided. In some embodiments, the therapeutic agent comprises a RNA or DNA, and in some aspects, the RNA is an mRNA. In further aspects, the mRNA encodes a nuclease or transcription factor. In some embodiments, the pharmaceutical compositions further comprise one or more components selected from neutral lipids, charged lipids, steroids and polymer conjugated lipids. Such compositions are useful for formation of lipid nanoparticles for the delivery of the therapeutic agent.

In still further embodiments, the LNPs comprise a pegylated lipid having the following structure (V):

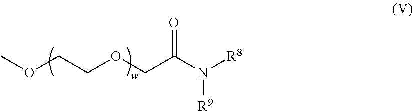

or a pharmaceutically acceptable salt, tautomer or stereoisomer thereof, wherein R⁸, R⁹ and w are as defined herein for Formula (V).

Thus, in one embodiment, described herein is a LNP comprising a cationic lipid, wherein the cationic lipid is selected from a lipid of Formula I, II, III and IV, and optionally a pegylated lipid of Formula V, wherein the LNP also comprises one or more nucleic acids (e.g., nucleic acids encoding one or more engineered nucleases and/or donor (transgene) molecules).

In some embodiments, the nucleic acid encodes an engineered nuclease wherein the nuclease comprises a DNA binding domain and a cleavage domain. In other embodiments, the nucleic acid of the LNP encodes an engineered transcription factor comprising a DNA binding domain and transcriptional domain (e.g., activation or repression domain). In some aspects, the DNA binding domain is a zinc finger DNA binding domain, a TALE DNA binding domain, a RNA molecule (e.g., single guide (sg) RNA of a CRISPR/Cas nuclease), or a meganuclease DNA binding domain. In further aspects, the cleavage domain of the nuclease comprises a wild-type or an engineered (mutated) cleavage domain from an endonuclease, a meganuclease DNA cleavage domain, or a Cas DNA cleavage domain. In some embodiments, the DNA cleavage domain is FokI. In further embodiments, the FokI domain comprises mutations in the dimerization domain (see e.g. U.S. Pat. No. 8,623,618) or in the regions of the FokI domain that non-specifically interact with the phosphate backbone of the DNA molecule (See e.g. U.S. patent application Ser. No. 15/685,580).

In another aspect, fusion polypeptides comprising a DNA binding domain and an engineered cleavage half-domain as described herein are provided. In certain embodiments, the DNA-binding domain is a zinc finger binding domain (e.g., an engineered zinc finger binding domain). In other embodiments, the DNA-binding domain is a TALE DNA-binding domain. In still further embodiments, the DNA binding domain is a catalytically inactive Cas9 or Cfp1 protein (dCas9 or dCfp1). In some embodiments, the engineered cleavage half-domain forms a nuclease complex with a catalytically inactive engineered cleavage half-domain to form a nickase (see U.S. Pat. No. 9,200,266). In certain embodiments, the zinc finger domain recognizes a target site in an albumin gene or a globin gene in red blood cells (RBCs). See, e.g., U.S. Publication No. 2014001721, incorporated by reference in its entirety herein. In other embodiments, the ZFN, TALEN, and/or CRISPR/Cas system binds to and/or cleaves a safe-harbor gene, for example a CCR5 gene, a PPP1R12C (also known as AAVS1) gene, albumin, HPRT or a Rosa gene. See, e.g., U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20060063231; 20080159996; 201000218264; 20120017290; 20110265198; 20130137104; 20130122591; 20130177983; 20130177960 and 20140017212. The nucleases (or components thereof) may be provided as a polynucleotides encoding one or more ZFN, TALEN, and/or CRISPR/Cas system described herein. The polynucleotides may be, for example, mRNA. In some aspects, the mRNA may be chemically modified (See e.g. Kormann et al, (2011) Nature Biotechnology 29(2):154-157). In other aspects, the mRNA may comprise an ARCA cap introduced during synthesis (see U.S. Pat. Nos. 7,074,596 and 8,153,773). In some aspects the mRNA may comprise a cap introduced by enzymatic modification. The enzymatically introduced cap may comprise Cap0, Cap1 or Cap2 (See e.g. Smietanski et al, (2014) Nature Communications 5:3004). In further aspects, the mRNA may be capped by chemical modification. In further embodiments, the mRNA may comprise a mixture of unmodified and modified nucleotides (see U.S. Patent Publication 20120195936). In still further embodiments, the mRNA may comprise a WPRE element (see U.S. patent application Ser. No. 15/141,333), and in further embodiments, the WPRE element may be a mutated WPRE element (see e.g. Zanta-Boussif et al (2009) Gene Ther 16(5):605-19). In some embodiments, the mRNA is double stranded (See e.g. Kariko et al (2011) Nucl Acid Res 39:e142). In other embodiments the mRNA is single stranded. In some embodiments, the polyA track at the end of the message is extended. In preferred embodiments, the polyA track comprises 50 more polyAs, including for example, 50, 51 or 64 poly As. In more preferred embodiments, the polyA track comprises 128 poly As, or comprises 193 poly As or more. In preferred embodiments, the poly A track comprises 50, 51, 64, 70, 80, 90, 100, 110, 120, 128, 130, 140, 150, 160, 170, 180, 190, 193, 200 or more poly As. In other embodiments, some, most or all of the uridines in the wobble positions in the codons of the mRNAs are altered to another nucleotide. In some embodiments, 5%, 6%, 7%, 8%, 9%, 10%, 11%, 12%, 13%, 14%, 15% or more of the nucleotides in the wobble positions are altered.

The methods and compositions of the invention also include LNPs comprising ZFNs with mutations to amino acids within the ZFP DNA binding domain (“ZFP backbone”) that can interact non-specifically with phosphates on the DNA backbone, but they do not comprise changes in the DNA recognition helices. Thus, the invention includes mutations of cationic amino acid residues in the ZFP backbone that are not required for nucleotide target specificity. See, e.g., U.S. application Ser. No. 15/685,580.

In some embodiments, the LNPs comprise a donor molecule (e.g., DNA or RNA). In some aspects, the donor is a plasmid, minicircle or a linear DNA. In further embodiments, the LNPs comprise both RNAs and DNAs. In some aspects, the RNAs and/or DNAs encode fusion proteins, and in some instances, the fusion proteins are engineered nucleases or engineered transcription factors. RNAs and DNAs may be provided in any combination, including but not limited to, RNA nuclease(s) and DNA donors, RNA nucleases and donors, RNA donors and DNA nucleases, and DNA nucleases and donors. In some embodiments, the RNAs provided encode specific nucleases for cleaving an endogenous gene, and the DNAs provided comprise transgene cassettes for insertion into the cleaved gene. The DNA or RNA transgenes can comprise an open reading frame encoding a therapeutic protein or mRNA of interest, and can further comprise regulatory sequences such as promoter sequences, and can still further comprise sequences associated with increased expression of the transgene including intron and enhancer sequences. In further embodiments, the promoter sequences have tissue specific expression patterns. In some embodiments, the DNA transgenes comprise homology arms to enhance nuclease-driven targeted integration. In other embodiments, the DNA or RNA transgene comprises a cDNA encoding a therapeutic protein, or may encode a RNA molecule of interest such as a shRNA, miRNA, RNAi etc. In further embodiments, the donor may be a cDNA that may comprise the full-length transgene, or may comprise a truncated or fragment of the transgene. In some embodiments, the transgene for insertion is a wild type gene for insertion into a cell that does not express a wild type version of the gene. In other embodiments, the transgene for insertion encodes an engineered therapeutic protein such as an antibody or a modified version of a therapeutic protein with improved qualities compared to the wild type version thereof.

Donor sequences can range in length from 50 to 5,000 nucleotides (or any integral value of nucleotides therebetween) or longer. In some embodiments, the donor comprises a full-length gene flanked by regions of homology with the targeted cleavage site. In some embodiments, the donor lacks homologous regions and is integrated into a target locus through homology independent mechanism (i.e. NHEJ). In other embodiments, the donor comprises a smaller piece of nucleic acid flanked by homologous regions for use in the cell (i.e. for gene correction via targeted integration). In some embodiments, the donor comprises a gene encoding a functional or structural component such as a shRNA, RNAi, miRNA or the like. In other embodiments, the donor comprises sequences encoding one or more regulatory elements that bind to and/or modulate expression of a gene of interest. In other embodiments, the donor is a regulatory protein of interest (e.g. ZFP TFs, TALE TFs and/or a CRISPR/Cas TF) that binds to and/or modulates expression of a gene of interest.

In some embodiments, the transgene encodes a protein for treatment of a patient in need thereof, and is integrated using the methods and compositions of the invention into a safe harbor locus. In some aspects, the safe harbor is selected from AAVS1, albumin, HPRT or Rosa. The transgene may encode a protein such that the methods of the invention can be used for production of protein that is deficient or lacking (e.g., “protein replacement”). In some instances, the protein may be involved treatment for a lysosomal storage disease. Other therapeutic proteins may be expressed, including protein therapeutics for conditions as diverse as epidermolysis bullosa or AAT deficient emphysema. In other aspects, the transgene may comprise sequences (e.g., engineered sequences) such that the expressed protein has characteristics which give it novel and desirable features (increased half-life, changed plasma clearance characteristics etc.). Engineered sequences can also include amino acids derived from the albumin sequence. In some aspects, the transgenes encode therapeutic proteins, therapeutic hormones, plasma proteins, antibodies and the like. In some aspects, the transgenes may encode proteins involved in blood disorders such as clotting disorders. In some embodiments, the protein is an engineered transcription factor, for example a repressor, for treatment of a neurological disorder (e.g., an Htt repressor for treatment of Huntington's). See, e.g., U.S. Pat. No. 9,234,016 and U.S. Publication No. 20150335708.

Also provided herein are cells that have been modified by the LNPs, virus, polypeptides and/or polynucleotides of the invention. In some embodiments, the cells comprise a nuclease-mediated insertion of a transgene, or a nuclease-mediated knock out of a gene. The modified cells, and any cells derived from the modified cells do not necessarily comprise the nucleases of the invention more than transiently, but the modifications mediated by such nucleases remain. Cells of the invention can be eukaryotic or prokaryotic cells. In some embodiments, eukaryotic cells can comprise, but are not limited to, mammalian cells, plant cells, stem cells, embryonic stem cells, hematopoietic stem cells, hepatic cells, pulmonary cells, muscle cells, cardiac cells, neuronal cells, skin cells, bone cells, gastrointestinal cells, kidney cells and tumor cells. The cells can also be fungal cells, primate cells, mouse cells and human cells.

In yet another aspect, methods for targeted cleavage of cellular chromatin in a region of interest; methods of causing targeted alterations (e.g., insertions and/or deletions) to occur in a cell; methods of treating infection; and/or methods of treating disease are provided. These methods may be practiced in vitro, ex vivo or in vivo. The methods involve cleaving cellular chromatin at a predetermined region of interest in cells by expressing a pair of fusion polypeptides as described herein (i.e., a pair of fusion polypeptides in which one fusion polypeptide comprises the engineered cleavage half-domains as described herein). In certain embodiments, the targeted cleavage of the on-target site is increased by at least 50 to 200% (or any value therebetween) or more, including 50%-60% (or any value therebetween), 60%-70% (or any value therebetween), 70%-80% (or any value therebetween), 80%-90% (or any value therebetween, 90% to 200% (or any value therebetween), as compared to cleavage domains without the mutations as described herein. Similarly, using the methods and compositions as described herein, off-target site cleavage is reduced by 1-100 or more fold, including but not limited to 1-50 fold (or any value therebetween).

Targeted alterations include, but are not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations. Alterations can also include conversion of base pairs that are part of a coding sequence such that the encoded amino acid is altered. Alternations can be facilitated by homology-directed repair mechanisms or non-homology directed repair mechanisms.

A composition comprising any of the LNPs described herein is also provided. In some embodiments, the composition comprises one or more types of LNPs where each LNP type comprises alternate nucleic acids and/or alternate cationic lipids. In some instances, the cationic lipids are described by any one of Formulas I-IV. In further embodiments, the LNPs can comprise a pegylated lipid as described by Formula V. In some instances, the composition further comprises a viral particle. In some aspects, the viral particle is an AAV, adenovirus, lentivirus or the like. In some embodiments, the virus comprises a DNA, while in further embodiments, the LNP comprises mRNAs and the virus comprises a DNA donor as described herein. Especially preferred are compositions comprising a LNP comprising mRNAs encoding a nuclease, and an AAV comprising a DNA donor. In some aspects, the nuclease is an engineered nuclease (e.g., a ZFN, TALEN, MegaTAL, meganuclease, or TtAgo or CRISPR/Cas system).

In some embodiments, the composition of the invention comprises additional compositions such as buffers, stabilizers, etc.

In some embodiments, the composition comprising the LNPs comprising mRNA encoding a fusion protein is administered to a patient in need thereof as a single dose. In other embodiments, the composition is administered to a patient 1, 2, 3 or more times. In some embodiments, the composition is administered to a patient and then re-administered 7, 14, 21, 28, 30, 40, 50, 75, 100, 200 or more days after the first administration. In further embodiments, additional administration is performed 1, 2, 5, or 10 years following the first administration, or following the one or more administrations done in the first year.

The LNPs of the invention can be used for treatment of a patient in need thereof. Thus, the invention comprises methods for treating a patient in need thereof wherein the method comprises formulating a composition comprising the LNPs of the invention and administering the composition to a patient such that a disease is prevented or treated. In other embodiments, the LNPs of the invention can be used for transduction of cells in vitro. Thus, the invention comprises methods for transducing a cell with LNPs, optionally formulated as a composition, to introduce the gene therapy reagents of the invention into the cell. The LNPs as described herein may be administered sequentially and/or repeatedly, including but not limited to, administration of an LNP nuclease before and/or after administration of an LNP donor.

In another aspect, described herein is a kit comprising LNPs comprising nucleic acids as described herein (e.g., a fusion protein as described herein or a polynucleotide encoding one or more zinc finger proteins, cleavage domains, transcriptional activation or repression domains and/or fusion proteins as described herein; virus comprising donors of interest as described herein, ancillary reagents; and optionally instructions and suitable containers. The kit may also include one or more nucleases or polynucleotides encoding such nucleases.

Thus, the methods and compositions of the invention comprise at least the following embodiments:

1. A lipid nanoparticle (LNP) comprising one or more polynucleotides having activity as a gene therapy reagent.

2. The LNP of 1, wherein a) one or more of the polynucleotides encode one or more engineered nucleases, one or more engineered transcription factors and/or one or more transgenes encoding therapeutic proteins, for example proteins deficient or lacking in a subject having a disorder such as a lysosomal storage disease or a clotting disorder, or b) wherein the polynucleotide encodes an antisense RNA.

3. The LNP of 1 or 2, wherein the polynucleotides are randomly integrated into the genome, integrated in a targeted manner into the genome or expressed episomally in a cell.

4. The LNP of 2 or 3, wherein the nuclease or the transcription factor comprises a zinc finger protein, a TAL-effector domain or a single guide RNA of a CRISPR/Cas system, the nuclease further comprises a wild-type or engineered (mutant) cleavage domain such as FokI or a Cas endonuclease domain and the transcription factor further comprises a transcriptional regulatory domain such as an activation domain or a repression domain.

5. The LNP of any of 1 to 4, wherein the polynucleotides comprise DNA and/or RNA.

6. The LNP of 4, wherein the RNA is mRNA and the DNA is a plasmid, a minigene, or a linear DNA.

7. The LNP of any of 1 to 6, comprising a first polynucleotide encoding a nuclease and a second polynucleotide comprising a transgene.

8. The LNP of 7, wherein expression of the nuclease in a cell results in targeted integration of the transgene into the genome of a cell.

9. The LNP of any of 1 to 8 comprising cationic lipid molecules and optionally neutral lipids, charged lipids, steroids including cholesterol and/or their analogs, and/or polymer conjugated lipids.

10. The LNP of 9, wherein the cationic lipid is selected from compounds having the following Formulas (I, II, III and IV):

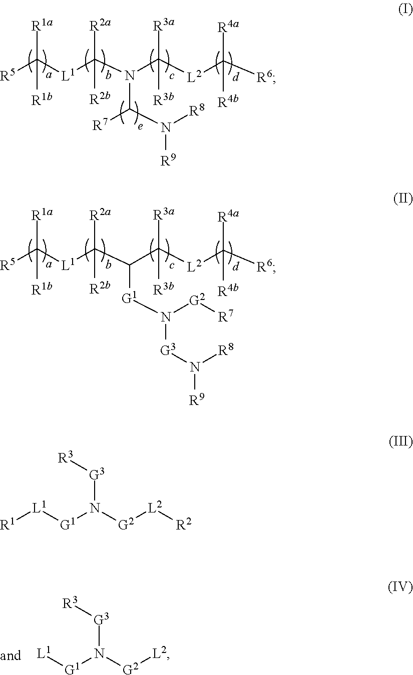

or a pharmaceutically acceptable salt, tautomer or stereoisomer thereof, wherein R¹, R², R³, R^{1a}, R^{1b}, R^{2a}, R^{2b}, R^{3a}, R^{3b}, R^{4a}, R^{4b}, R⁵, R⁶, R⁷, R⁸, R⁹, L¹, L², G¹, G², G³, a, b, c, d and e are as defined herein for each of Formulas (I), (II), (III) and (IV), including compounds I-5, II-6, II-9, II-11, II-36, III-25, III-45, III-49 and IV-12 shown below:

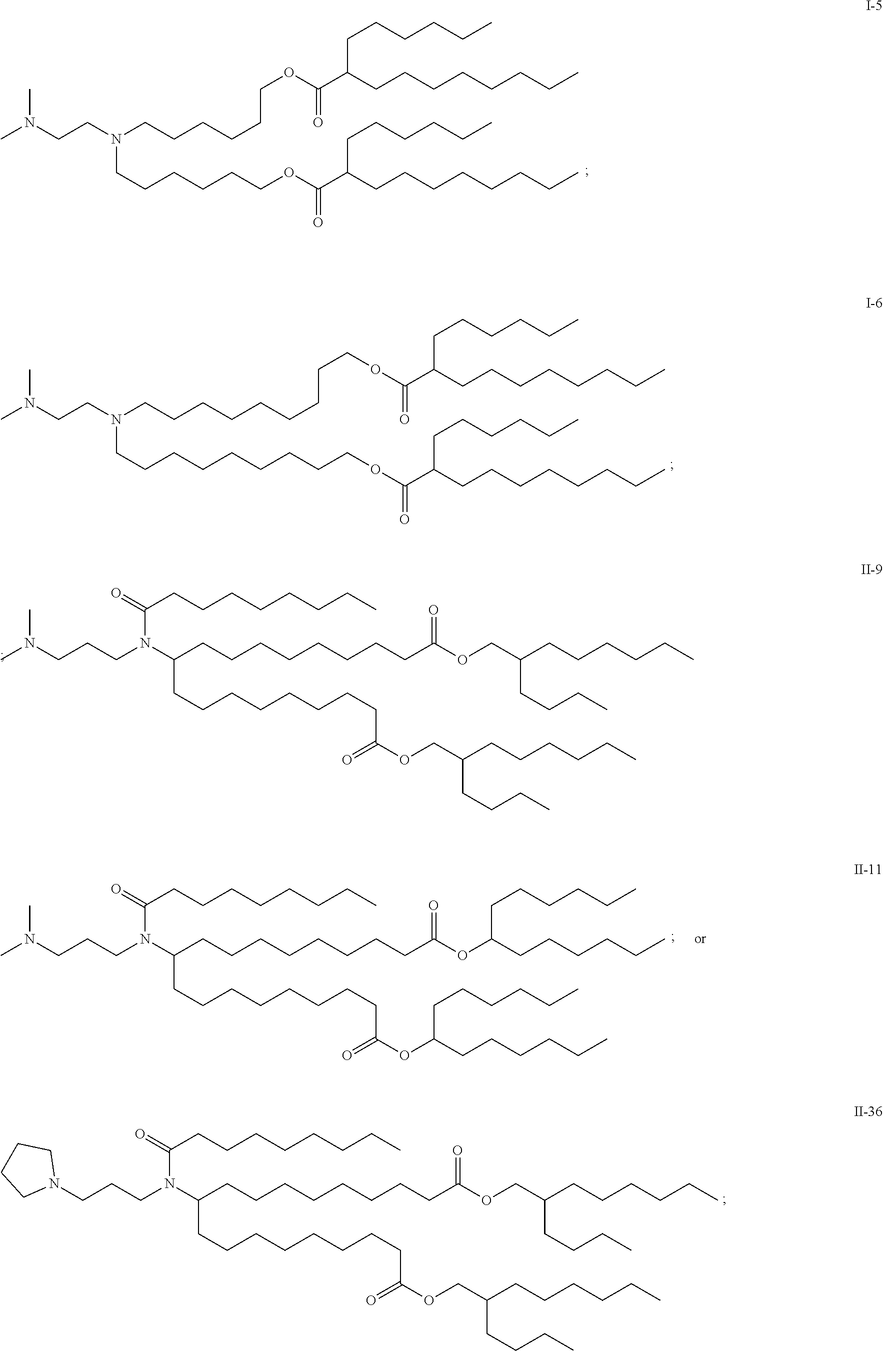

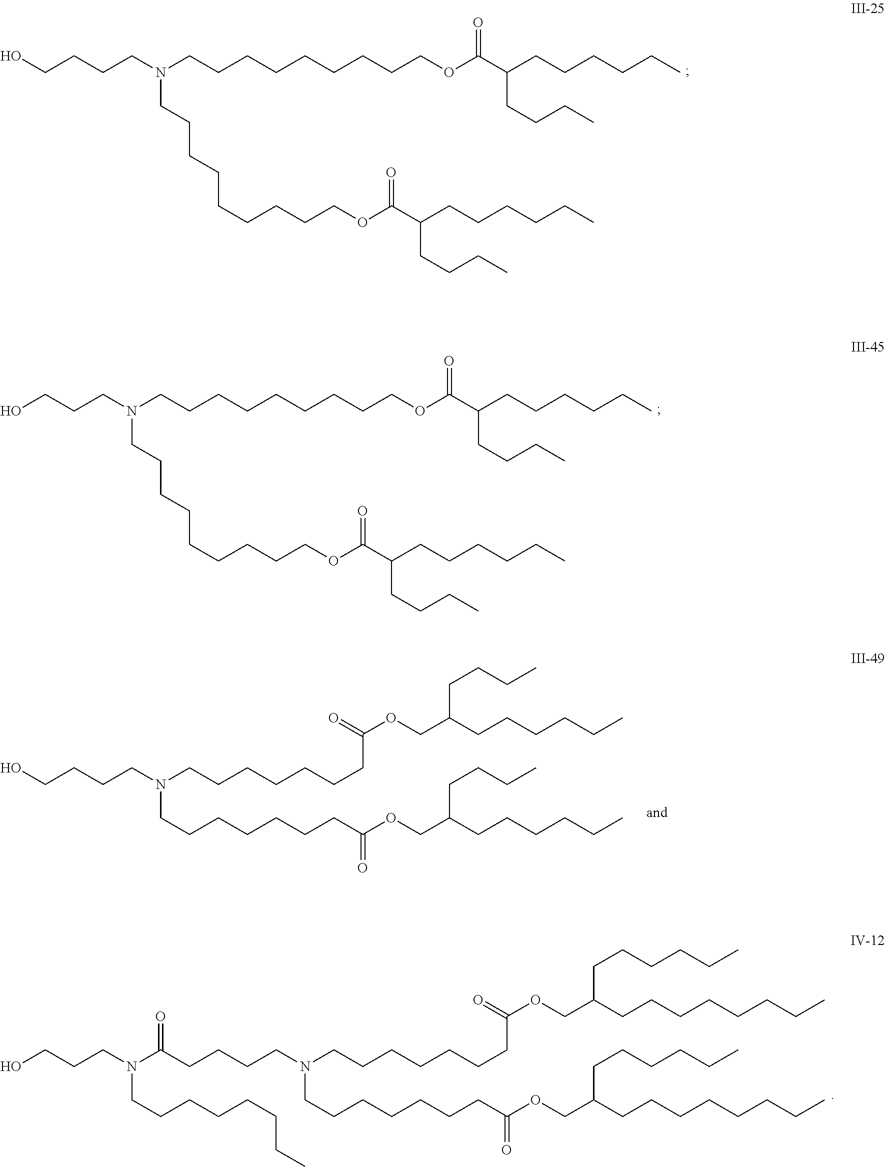 [SMILES: CCCCCCC(CCCC)C(=O)OCCCCCCCCCN(CCCCO)CCCCCCCCCOC(=O)C(CCCC)CCCCCC ; CCCCCCC(CCCC)C(=O)OCCCCCCCCCN(CCCO)CCCCCCCCCOC(=O)C(CCCC)CCCCCC ; CCCCCCC(CCCC)COC(=O)CCCCCCCN(CCCCO)CCCCCCCC(=O)OCC(CCCC)CCCCCC ; CCCCCCCCC(CCCCCC)COC(=O)CCCCCCCN(CCCCCCCC(=O)OCC(CCCCCC)CCCCCCCC)CCCCC(=O)N(CCCO)CCCCCCCC]

11. The LNP of any of 1 to 10, comprising a pegylated lipid having the following structure (V):

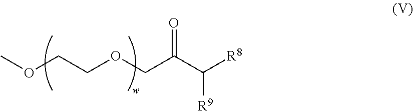

or a pharmaceutically acceptable salt, tautomer or stereoisomer thereof, wherein R⁸, R⁹ and w are as defined herein for Formula (V).

12. A pharmaceutical composition comprising one or more LNPs according to any of 1 to 11, optionally wherein the composition includes different LNPs.

13. A cell comprising one or more LNPs according to any of 1 to 11 or comprising a pharmaceutical composition according to 12 or a cell descended from the cell.

14. A genetically modified cell that has been modified by an LNP according to any of 1 to 11 or a cell descended from the genetically modified cell, wherein the genetic modifications include point mutations (i.e., conversion of a single base pair to a different base pair), substitutions (i.e., conversion of a plurality of base pairs to a different sequence of identical length), insertions of one or more base pairs, and/or deletions of one or more base pairs.

15. A method of delivering one or more polynucleotides to a cell or subject, the method comprising administering one or more LNPs according to any of 1 to 11 or a pharmaceutical composition according to 12.

16. A method of cleaving a region of interest in a cell, the method comprising delivering one or more LNPs according to any of 1 to 11 or a pharmaceutical composition of 12, wherein at least one LNP comprises a polynucleotide encoding a nuclease that cleaves the region of interest.

17. The method of 16, wherein the region of interest is in a safe harbor gene, optionally an AAVS1 gene, an albumin gene, a Rosa gene, a CCR5 gene, a CXCR gene or an HPRT gene.

18. The method of any of 15 to 17, wherein the one or more LNPs comprises a donor comprising a transgene and the transgene is integrated into the genome of the cell following cleavage by the nuclease.

19. A method of treating a patient in need thereof, the method comprising administering one or more LNPs according to the method of any of 15 to 18.

20. The method of any of 15 to 19, wherein the method is performed in vitro, ex vivo, or in vivo.

21. The method of any of 15 to 20, wherein the LNPs and/or pharmaceutical composition is administered one or more times, optionally, 1, 2, 3 or more times.

22. The method of any of 21, wherein the LNPs and/or pharmaceutical composition is administered to a patient and re-administered 7, 14, 21, 28, 30, 40, 50, 75, 100, and/or 200 or more days after the initial administration.

23. Use of one or more LNPs according to any of 1 to 11 or a pharmaceutical composition of 12 for the treatment of a disorder in a subject.

24. The use of 23, wherein the polynucleotide of the LNP encodes a transcriptional regulator that modulates expression of a target gene in the subject.

25. The use of 23, wherein at least one of the polynucleotides of the LNP encodes a therapeutic protein lacking or deficient in the subject and optionally a polynucleotide of the LNP encodes a nuclease.

26. The use of 25, wherein the therapeutic protein is integrated into the genome via nuclease-mediated targeted integration, optionally into an AAVS1 gene, an albumin gene, a Rosa gene, a CCR5 gene, a CXCR gene or an HPRT gene.

27. The use of any of 23 to 26, wherein the LNPs and/or pharmaceutical composition is administered one or more times, optionally, 1, 2, 3 or more times.

28. The method of any of 21, wherein the LNPs and/or pharmaceutical composition is administered to a patient and re-administered 7, 14, 21, 28, 30, 40, 50, 75, 100, and/or 200 or more days after the initial administration.

29. A kit comprising one or more LNPs according to 1 to 11 or pharmaceutical composition according to 12.

These and other aspects will be readily apparent to the skilled artisan in light of disclosure as a whole.

### BRIEF DESCRIPTION OF THE DRAWINGS

FIGS. 1A through 1F are graphs showing the results of in vivo administration of LNPs comprising nucleic acids encoding exemplary ZFNs. FIG. 1A depicts single doses of separate (individual) 30724 and 30725 ZFN mRNA (dual HBB 3′ UTR; 25% 2 thiouridine (2tU) and 25% 5 methyl cytosine (5mC) nucleoside substitution; ARCA-capped; silica-purified) mixed together and formulated into LNP formulation comprising cationic lipid I-6 and injected into mice at a range of doses and livers harvested. Animals for this study were not pre-treated with dexamethasone. FIG. 1B shows the results for doses of separate 30724 and 30725 ZFN mRNA (dual HBB 3′ UTR; 25% 2tU & 25% 5mC nucleoside substitution; ARCA-capped; silica-purified) mixed together and formulated into LNP formulation comprising cationic lipid I-6 and injected into mice at 3 mg/kg and livers harvested. Animals for this study were not pre-treated with dexamethasone. FIG. 1C shows the results for single doses of either 30724 and 30725 (separate mRNAs) or 48641 and 31523 (separate mRNAs) ZFN mRNA (dual HBB 3′ UTR; 25% 2tU & 25% 5mC nucleoside substitution; ARCA-capped; silica-purified) mixed together and formulated into LNP formulation I-6 and injected into mice at 2 mg/kg and livers harvested. Animals were not pre-treated with dexamethasone. FIG. 1D shows the results of single doses of one 2A-linked 48641 and 31523 ZFN mRNA (25% 2tU & 25% 5mC nucleoside substitution; ARCA-capped; silica-purified) with either the dual HBB or WPRE 3′ UTR mixed together and formulated into LNP formulation comprising cationic lipid I-6 and injected into mice at 2 mg/kg and livers harvested. Animals were not pre-treated with dexamethasone. FIG. 1E depicts the results for single doses of separate 48641 and 31523 ZFN mRNA (WPRE 3′ UTR; 25% 2tU & 25% 5mC nucleoside substitution; ARCA-capped; silica-purified) mixed together and formulated into LNP formulation comprising cationic lipid I-6 or I-5 and injected into mice at 2 mg/kg and livers harvested. Animals were not pre-treated with dexamethasone. FIG. 1F shows the results for single doses of separate 48641 and 31523 ZFN mRNA (WPRE 3′ UTR; 25% 2tU & 25% 5mC nucleoside substitution; ARCA-capped; silica-purified) mixed together and formulated into LNP formulation comprising cationic lipid I-5 and injected into mice at a range of doses and livers harvested. Animals were not pre-treated with dexamethasone. For all data shown, “Dual HBB 3′ UTR” refers to two copies of the 3′ untranslated region (UTR) of the human beta globin gene (see Example X for details); “25% 2tU” refers to mRNA comprising 25% pseudo uridine; “25% 5 mM C” refers to mRNA comprising 25% methyl cytidine; “ARCA-capped” refers to mRNA comprising ARCA (anti-reverse Cap Analog) caps; “silica-purified” refers to the method used to purify the mRNAs (see Example 2); “WPRE” means a RNA structure known as a woodchuck posttranscriptional regulatory element where the mRNAs comprised a WPRE stem loop structure for added stability. In all graphs, the individual data points represent individual mice. The data demonstrates that LNPs comprising the ZFN pairs 30724/30725 and 48641/31523 were able to induce cleavage in the mouse livers when formulated with either the I-6 or I-5 cationic lipid.

FIGS. 2A through 2D are graphs comparing the results for the I-5 and II-9 cationic lipid containing LNPs and further optimization studies for the nuclease-encoding mRNAs. FIG. 2A shows the nuclease activity for treatment with LNPs comprising mRNAs encoding ZFNs 48641 and 31523 (the mRNAs comprised WPRE 3′ UTR; 25% 2tU & 25% 5mC nucleoside substitution; ARCA-capped; silica-purified) mixed together and formulated into LNP formulation comprising cationic lipids I-5 or II-9 and injected into mice at 1 or 3 mg/kg and livers harvested. Animals were not pre-treated with dexamethasone. FIG. 2B shows the results for repeat dosing (28 day intervals) of separate 48641 and 31523 ZFN mRNA (WPRE 3′ UTR; 25% 2tU & 25% 5mC nucleoside substitution; ARCA-capped; silica-purified) mixed together and formulated into LNP formulation comprising cationic lipid II-9 and injected into mice at 2 mg/kg and livers harvested. Animals were not pre-treated with dexamethasone. FIG. 2C shows the results for single doses of separate 48641 and 31523 ZFN mRNA (WPRE 3′ UTR; 25% 2tU & 25% 5mC or 25% pU nucleoside substitution; ARCA-capped; silica-purified) mixed together and formulated into an LNP formulation comprising cationic lipid II-9 and injected into mice at 2 mg/kg and livers harvested. Animals were not pre-treated with dexamethasone. FIG. 2D shows the result for Repeat dosing (14 day intervals) of separate 48641 and 31523 ZFN mRNA (WPRE 3′ UTR; 25% pU nucleoside substitution; ARCA-capped; silica-purified) mixed together and formulated into an LNP formulation comprising cationic lipid II-9 and injected into mice at 2 mg/kg and livers harvested. Animals were not pre-treated with dexamethasone.

FIGS. 3A through 3C are graphs depicting results from experiments to optimize purification schemes and different mRNA compositions and caps. FIG. 3A shows repeat dosing (14-day intervals) of individual 48641 and 31523 ZFN mRNA (WPRE 3′ UTR; 25% pU nucleoside substitution) mixed together and formulated into LNP formulation comprising cationic lipid II-9 and injected into mice at 3.5 mg/kg and livers harvested. Animals were pre-treated with dexamethasone. FIG. 3B shows the results for repeat dosing (14 day intervals) of individual 48641 and 31523 ZFN mRNA (WPRE 3′ UTR; 25% pU nucleoside substitution; Cap1) mixed together and formulated into an LNP formulation comprising cationic lipid II-9 and injected into mice at 2 mg/kg and livers harvested. Animals were pre-treated with dexamethasone. FIG. 3C depicts the results from repeat dosing (14-day intervals) of individual 48641 and 31523 ZFN mRNA (WPRE 3′ UTR; 25% pU nucleoside substitution (open circles) or no pU substitution (closed circles); silica-purified comprising the different caps indicated) mixed together and formulated into an LNP formulation comprising cationic lipid II-9 and injected into mice at 2 mg/kg and livers harvested. Animals were pre-treated with dexamethasone.

FIGS. 4A and 4B are graphs depicting the results from experiments testing the effect of immunosuppression on the activity of the nucleases delivered via LNPs. FIG. 4A shows the results from repeat dosing (14-day intervals) of individual 48641 and 31523 ZFN mRNA (WPRE 3′ UTR; 25% pU nucleoside substitution; ARCA-capped; silica-purified) mixed together and formulated into an LNP formulation comprising cationic peptide II-9 and injected into mice at 2 mg/kg and livers harvested. FIG. 4B shows the results of single doses of individual (separate) 48641 and 31523 ZFN mRNA (WPRE 3′ UTR; Cap1; silica-purified) mixed together and formulated into LNP formulation comprising cationic lipid II-9 and injected into mice at 2 mg/kg and livers harvested. Animals were either pre-treated with dexamethasone or treated with Solu-medrol® (methylprednisolone sodium succinate) one day prior to LNP dosing and then daily for 3 additional days.

FIGS. 5A and 5B are graphs depicting the in vitro activity of the nucleases delivered via LNP. FIG. 5A shows nuclease activity from single doses of individual 48641 and 31523 ZFN mRNA (WPRE 3′ UTR; wild-type resides or 25% pU nucleoside substitution; ARCA-capped or Cap1 as indicated; silica-purified) mixed together and formulated into an LNP formulation comprising cationic lipid II-9 and added into Hepa1-6 cell culture at a range of doses. FIG. 5B shows nuclease activity from single doses of individual 48641 and 31523 ZFN mRNA (murine Albumin), 59771 and 59790 ZFN mRNA (murine TTR), or 58780 and 61748 ZFN mRNA (murine PCSK9) (WPRE 3′ UTR; 25% pU nucleoside substitution; Cap1; silica-purified) mixed together and formulated into an LNP formulation comprising cationic lipid II-9 and added into Hepa1-6 liver cell culture at a range of doses.

FIGS. 6A through 6D depict the results from in vivo dosing of nucleases targeting murine albumin or TTR delivered via LNP. FIG. 6A shows the results of repeat dosing (14-day intervals for a total of 4 doses) of individual 48641 and 31523 ZFN mRNA (murine Albumin) or 59771 and 59790 ZFN mRNA (murine TTR) (WPRE 3′ UTR; 25% pU nucleoside substitution; ARCA-capped; silica-purified) mixed together and formulated into an LNP formulation comprising cationic lipid II-9 and injected into mice at 0.8 mg/kg and livers harvested after 2 or 4 doses. Animals were pre-treated with dexamethasone. FIG. 6B shows the results of an ELISA to determine TTR in the plasma following treatment. Plasma was collected from mice described in FIG. 6A. FIG. 6C shows the results from repeat dosing (14-day intervals for a total of 4 doses) of individual 48641 and 31523 ZFN mRNA (murine Albumin) or 58780 and 61748 ZFN mRNA (murine PCSK9) (WPRE 3′ UTR; 25% pU nucleoside substitution; ARCA-capped; silica-purified) mixed together and formulated into an LNP formulation comprising cationic lipid II-9 and injected into mice at 0.8 mg/kg and livers harvested after 2 or 4 doses. Animals were pre-treated with dexamethasone. FIG. 6D shows the results of an ELISA to determine PCSK9 in the plasma following treatment. Plasma was collected from mice described in FIG. 6C.

FIGS. 7A and 7B are graphs depicting the results of targeted integration of an IDS gene into the albumin locus. FIG. 7A shows the activity of the nucleases. Single doses of individual 48641 and 31523 ZFN mRNA (WPRE 3′ UTR; 25% pU nucleoside substitution; Cap1; silica-purified) mixed together and formulated into an LNP formulation comprising the cationic lipid II-9 and injected into mice at a range of doses along with 1.5e12 vector genomes (vg) AAV8 encoding a human IDS transgene donor. The IDS transgene comprised homology arms flanking the ZFN cut site in the mouse albumin gene and a splice acceptor just upstream of the transgene coding region. Animals were pre-treated with dexamethasone. Livers were harvested for indel analysis for determination of nuclease activity. FIG. 7B is a graph depicting IDS activity in the mice shown in FIG. 7A. In both graphs, every data point represents an individual mouse.

FIGS. 8A through 8F are graphs from additional studies using the LNPs for nuclease activity using the IVPRP approach for transgene integration. FIG. 8A shows the results of single doses of individual 48641 and 31523 ZFN mRNA (WPRE 3′ UTR; 25% 2tU & 25% 5mC nucleoside substitution; ARCA-capped; silica-purified) mixed together and formulated into an LNP formulation comprising cationic lipid I-5 and injected into mice at 2 mg/kg either 1 day after (pre-delivery) or at the same time as (co-delivery) 1.5e12 vector genomes (vg) AAV6 or AAV8 encoding a human IDS transgene donor with homology arms flanking the ZFN cut site and a splice acceptor just upstream of the transgene coding region. Animals were pre-treated with dexamethasone. Livers were harvested for indel analysis. FIG. 8B is a graph depicting IDS activity in plasma collected from the mice described in FIG. 8A. FIG. 8C shows the results of single doses of individual 48641 and 31523 ZFN mRNA (WPRE 3′ UTR; 25% 2tU & 25% 5mC or 25% pU nucleoside substitution; ARCA-capped; silica-purified) mixed together and formulated into an LNP formulation comprising cationic lipid I-5 or II-9 and injected into mice at 2 mg/kg at the same time as 1.5e12 vector genomes (vg) AAV8 encoding a human IDS transgene donor. The donor comprised homology arms flanking the ZFN cut site in the albumin gene and a splice acceptor just upstream of the transgene coding region. Animals were pre-treated with dexamethasone. Livers were harvested for indel analysis. FIG. 8D is a graph of IDS activity found in the plasma of the mice described in FIG. 8C. FIG. 8E is a graph showing the nuclease activity results from single doses of individual 48641 and 31523 or 48652 and 31527 ZFN mRNA (WPRE 3′ UTR; unmodified residues or 25% pU nucleoside substitution; ARCA-capped or Cap1; silica- or HPLC-purified) mixed together and formulated into an LNP formulation comprising cationic lipid II-9 and injected into mice at 2 mg/kg at the same time as 1.5e12 vector genomes (vg) AAV8 encoding a human IDS transgene donor. The donor had homology arms flanking the ZFN cut site in the albumin gene and a splice acceptor just upstream of the transgene coding region. Animals were pre-treated with dexamethasone. Livers were harvested for indel analysis. FIG. 8F is a graph showing the IDS activity in the plasma collected from the mice described in FIG. 8E.

FIGS. 9A and 9B are graphs depicting the results of the IVPRP approach with LNP nuclease delivery. FIG. 9A shows the nuclease activity results using single doses of individual 48641 and 31523 ZFN mRNA (WPRE 3′ UTR; unmodified residues or 25% pU nucleoside substitution; Cap1; silica-purified) mixed together and formulated into an LNP formulation comprising cationic lipid II-9 and injected into mice at 2 mg/kg at the same time as 1.5e12 vector genomes (vg) AAV8 encoding a human IDS transgene donor. The donor comprised homology arms flanking the ZFN cut site in the albumin gene and a splice acceptor just upstream of the transgene coding region. Animals were either pre-treated with dexamethasone just prior to LNP dosing or just prior to and for an additional 3 days after dosing. Livers were harvested for indel analysis. FIG. 9B shows the IDS activity in plasma was collected from the mice described in FIG. 9A.

FIGS. 10A and 10B are graphs depicting the results using the IVPRP approach varying the cationic lipid in the LNP formulation and using a Factor IX (FIX) donor. FIG. 10A shows the nuclease activity results for single doses of individual 48641 and 31523 ZFN mRNA (WPRE 3′ UTR; 25% pU nucleoside substitution; ARCA-capped; silica-purified) mixed together and formulated into an LNP formulation comprising cationic lipid II-9 or I-5 and injected into mice at 2 or 3.5 mg/kg at the same time as 1.5e12 vector genomes (vg) AAV8 encoding a human FIX transgene donor. The FIX donor comprised homology arms flanking the ZFN cut site and a splice acceptor just upstream of the transgene coding region. Animals were pre-treated with dexamethasone just prior to LNP dosing. Livers were harvested for indel analysis. FIG. 10B shows the FIX activity results in plasma collected from the mice described in FIG. 10A.

FIGS. 11A through 11D are graphs depicting the results using repeat dosing in the IVPRP approach. FIG. 11A is a graph showing nuclease activity following repeat dosing (14-day intervals) of individual 48641 and 31523 ZFN mRNA (WPRE 3′ UTR; 25% pU nucleoside substitution; ARCA-capped; silica-purified) mixed together and formulated into an LNP formulation comprising cationic lipid II-9 and injected into mice at 2 mg/kg. The first dosing also included co-delivery of 1.5e12 vector genomes (vg) AAV8 encoding a human IDS transgene donor with homology arms flanking the ZFN cut site in the albumin gene and a splice acceptor just upstream of the transgene coding region. Animals were pre-treated with dexamethasone prior to each LNP dosing. Livers were harvested for indel analysis. FIG. 11B is a graph showing the IDS activity detected in plasma was collected from the mice described in FIG. 11A. FIG. 11C is a graph depicting the nuclease activity results of repeat dosing (7 day intervals) of individual 48641 and 31523 ZFN mRNA (WPRE 3′ UTR; 25% pU nucleoside substitution; ARCA-capped; silica-purified) mixed together and formulated into an LNP formulation comprising cationic lipid II-9 and injected into mice at 2 mg/kg. The first dosing also included co-delivery of 1.5e12 vector genomes (vg) AAV8 encoding a human IDS transgene donor with homology arms flanking the ZFN cut site in the albumin gene and a splice acceptor just upstream of the transgene coding region. Animals were pre-treated with dexamethasone prior to each LNP dosing. Livers were harvested for indel analysis. FIG. 11D shows IDS activity in plasma that was collected from the mice described in FIG. 11C.

FIG. 12 is a graph showing nuclease activity in vivo following single doses of individual 48641 and 31523 ZFN mRNA (WPRE 3′ UTR; unmodified residues; Cap1; HPLC-purified) mixed together and formulated into an LNP formulation comprising cationic lipid II-9 or I-5 and injected into mice at a range of doses. Animals were not pre-treated with dexamethasone. Skin surrounding the injection site was harvested for indel analysis.

FIG. 13 is a graph showing the results of repeat doses of individual 48641 and 31523 ZFN mRNA (WPRE 3′ UTR; 25% pU nucleoside substitution; Cap1; silica-purified) mixed together and formulated into LNP formulations II-9, II-11, or III-45 and injected into mice at 1 mg/kg and livers harvested 7 days later. Animals (all female, not fasting) were pre-treated with dexamethasone (5 mg/kg, 30 minutes prior to LNP dosing).

FIGS. 14A through 14C are graphs depicting data following single doses of the LNPs comprising the ZFN, assaying for cleavage activity and transgene expression as well as liver function. FIG. 14A shows the data for single doses of individual 48641 and 31523 ZFN mRNA (WPRE 3′ UTR; 25% pU nucleoside substitution; Cap1; silica-purified) mixed together and formulated into LNP formulation II-9 and injected into mice at a range of doses along with 1.5e12 vector genomes (vg) AAV8 encoding a human IDS transgene donor with homology arms flanking the ZFN cut site and a splice acceptor just upstream of the transgene coding region. Animals were pre-treated with dexamethasone. Livers were harvested for indel analysis 28 days post-dosing. FIG. 14B shows IDS activity assay in plasma collected from the mice described in FIG. 14A. FIG. 14C shows results of liver function test (LFT) in serum collected from the mice described in FIG. 14A one day post-dosing. “LFT” refers toliver function test; “ALT” refers to alanine transaminase; “AST” refers to aspartate transaminase. The results demonstrated dose-dependent cleavage in vivo as well as dose dependent expression of the IDS transgene. Additionally, treatment of the animals with the LNPs did not cause any notable changes in liver function.

FIG. 15 is a graph depicting dose-dependent activity of the IDS transgene. Single doses of individual 48641 and 31523 ZFN mRNA (WPRE 3′ UTR; 25% pU nucleoside substitution; Cap1; silica-purified) mixed together and formulated into LNP formulation II-9 and injected into mice at 0.5 mg/kg along with AAV8 encoding a human IDS transgene donor with homology arms flanking the ZFN cut site and a splice acceptor just upstream of the transgene coding region at a range of vector genome (vg) doses. Animals were pre-treated with dexamethasone. Plasma was collected 28 days post-dosing and analyzed for IDS activity assay. The data demonstrated that the amount of IDS activity measured in the plasma displayed an AAV IDS transgene donor dose-dependent response.

FIG. 16 is a graph depicting results following 6 repeat administrations of II-9 cationic lipid containing LNPs comprising mRNAs encoding ZFNs 48641 and 31523 (the mRNAs comprised WPRE 3′ UTR; 25% pU nucleoside substitution; Cap1) that were mixed together and formulated into LNP formulation and injected into mice at 0.5 (silica-purified mRNA used, shown in closed squares) or 2 (HPLC-purified mRNA used, shown by open circles) mg/kg and livers harvested. Animals were pre-treated with dexamethasone (5 mg/kg, 30 minutes prior to LNP dosing). Control animals are shown as “PBS” (closed circles).

FIGS. 17A and 17B are graphs depicting biodistribution of genome modification following a single administration of II-9 cationic lipid containing LNPs comprising mRNAs encoding ZFNs 48641 and 31523 (the mRNAs comprised WPRE 3′ UTR; 25% pU nucleoside substitution; Cap1, silica-purified) that were mixed together and formulated into LNP formulation and injected into mice at 2.0 mg/kg and livers, bone marrow, and spleens harvested. Animals were pre-treated with dexamethasone (5 mg/kg, 30 minutes prior to LNP dosing). FIG. 17A shows genome modification (indels) in the indicated organs (liver, spleen, bone marrow). FIG. 17B shows genome modification of mice which were either sacrificed and unmanipulated prior to liver harvest (unperfused) or perfused transcardially with buffered saline prior to liver harvest to remove blood cells within the liver (unsorted). A fraction of the perfused liver was digested with collagenase to create a single cell suspension, then fluorescently immunostained with a kupffer cell-specific marker and an endothelial cell-specific marker. Stained cells were then FACS-sorted into endothelial cell marker positive, kupffer cell marker positive, or marker negative (hepatocyte) cell populations. Genomic DNA was then harvested from these sorted cells and analyzed for genome modification (indels).

FIG. 18 is a graph depicting genome modification (% indels) following a single administration of II-9 cationic lipid containing LNPs comprising 25% pU substituted or unmodified mRNAs encoding ZFNs 48641 and 31523 (the mRNAs comprised WPRE 3′ UTR; Cap1, silica-purified) that were mixed together and formulated into LNP formulation and injected into mice at 0.5 mg/kg and livers harvested. Animals were pre-treated with dexamethasone (5 mg/kg, 30 minutes prior to LNP dosing). Genomic DNA was then harvested and analyzed for genome modification (indels) as described in Example 2.

FIG. 19 is a graph depicting genome modification following a single administration of II-9 cationic lipid containing LNPs comprising 25% pU substituted or unmodified mRNAs encoding ZFNs 48641 and 31523 (the mRNAs comprised WPRE 3′ UTR; Cap1, silica-purified) that were mixed together and formulated into LNP formulation and injected into mice at 0.5 mg/kg and livers harvested. Animals were pre-treated with dexamethasone (5 mg/kg, 30 minutes prior to LNP dosing). Genomic DNA was then harvested and analyzed for genome modification (indels) as described in Example 2.

FIGS. 20A through 20C are graphs depicting cleavage activity and transgene expression data following single or multiple doses of the LNPs comprising the ZFN and a single dose of AAV comprising the human IDS transgene donor. FIG. 20A shows the data for single and multiple doses of individual 48641 and 31523 ZFN mRNA (WPRE 3′ UTR; unmodified residues; Cap1; HPLC-purified) mixed together and formulated into LNP formulation II-9 and injected into mice at 0.5 mg/kg along with 1.5e12 vector genomes (vg) AAV8 or AAV6 encoding a human IDS transgene donor with homology arms flanking the ZFN cut site and a splice acceptor just upstream of the transgene coding region. Animals were pre-treated with dexamethasone. Groups which were repeat dosed with LNP were dosed in 7-day intervals. Livers were harvested for indel analysis 35 days post-initial dosing. FIG. 20B shows IDS activity assay in plasma of subjects treated under the indicated conditions. FIG. 20C shows IDS activity assay in the indicated tissues (liver, spleen, kidney, marrow and brain) under the indicated conditions, collected from the mice described in FIG. 20A. The results demonstrated dose-dependent cleavage in vivo as well as dose dependent expression of the IDS transgene.

FIG. 21 is a graph depicting results from optimization studies using different mRNA compositions and polyA lengths. FIG. 21 shows repeat dosing (14-day intervals) of individual 48641 and 31523 ZFN mRNA (WPRE 3′ UTR; unmodified residues; Cap1; silica-purified), containing different polyA lengths and composition of uridines in the protein coding sequence as indicated, mixed together and formulated into LNP formulation comprising cationic lipid II-9 and injected into mice at 0.5 mg/kg and livers harvested 7 days post-dosing. “64polyA,” “128polyA,” and “193polyA refer, respectively, to 64, 128 or 193 long polyA regions while “uridine-depleted” refers to polynucleotides with at a percentage of uridines deleted from the wobble positions in the codons (see Examples). Geometric shapes in the graph indicate individual subjects. Animals were pre-treated with dexamethasone. The results demonstrate that a longer polyA tail and depleting the construct of as many uridines as possible while retaining the same amino acid sequence of the resulting translated protein yields the highest levels of gene modification.

FIGS. 22A through 22F are graphs depicting cleavage activity, protein knockdown, liver function and inflammatory cytokine secretion following single or multiple doses of LNPs comprising various ZFNs targeting exon 2 of the murine TTR gene. FIG. 22A shows the on-target cleavage data for single doses of individual 69121/69128 and 69052/69102 ZFN mRNA (WPRE 3′ UTR; unmodified residues; Cap1; silica-purified; 193 polyA tail; uridine-depleted) mixed together and formulated into LNP formulation II-9 and injected into mice at a range of doses. Animals were pre-treated with dexamethasone. Livers were harvested for indel and heparinized plasma harvested for protein knockdown analysis 35 days post-initial dosing. FIG. 22B shows murine TTR ELISA assay in plasma collected from the mice described in FIG. 22A. FIGS. 22C and 22D shows results of liver function test (LFT) in serum collected from the mice described in FIG. 22A one-day post-dosing. “LFT” refers to liver function test; “ALT” refers to alanine transaminase; “AST” refers to aspartate transaminase. FIGS. 22E and 22F are graphs showing genome editing in off-target organs spleen and kidney, respectively, collected at the same time as livers from 22A. The results demonstrated dose-dependent on-target, with minimal off-target, cleavage in vivo as well as dose dependent knockdown of mTTR protein expression. Additionally, treatment of the animals with the LNPs did not cause any notable changes in liver function.

FIG. 23 depicts a graph showing the results of repeat doses of individual 48641 and 31523 ZFN mRNA (WPRE 3′ UTR; 25% pU nucleoside substitution; Cap1; silica-purified) mixed together and formulated at an intermediate amino lipid (N):mRNA (P) ratio into LNP formulations II-9, II-36, III-25, III-45, III-49, or IV-12 and injected into mice at 0.5 mg/kg and livers harvested 7 days later. Formulation II-9 was also formulated at low and high N:P ratios as well as at an intermediate N:P ratio but a larger LNP of ˜100 nm. “N:P ratios” are the ratios of nitrogen (N) to phosphate (P) in the composition. N represents the nitrogen in the charged lipid while P represents the phosphate on the nucleic acid backbone. Thus, “high N:P” formulations have more lipid to nucleic acid than low N:P formulations. Animals (all male, fasted overnight prior to injection) were pre-treated with dexamethasone (5 mg/kg, 30 minutes prior to LNP dosing).

### DETAILED DESCRIPTION

Disclosed herein are methods and compositions for delivery of nucleases to cells. These methods comprise the use of novel lipid nanoparticles (LNP) comprising cationic lipids and optionally pegylated lipids. Embodiments of the LNPs are used to deliver mRNAs encoding the nucleases where the mRNAs can comprise a variety of caps (ARCA, Cap1, Cap2 or Cap0) and/or a variety of nucleoside compositions in the mRNA sequence. The LNPs comprising the ZFN-encoding mRNAs can be used to treat cells in vitro and in vivo. In animals, the LNPs can be co-dosed with a donor such that the nuclease delivered via the LNP can directed targeted integration of the donor.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

A “gene therapy reagent” or “gene therapy polynucleotide” is a reagent used to modulate gene expression in a cell. Gene therapy reagents can comprise nucleases and transcription factors where the reagents interact with a gene to modulate its expression. In some embodiments, the nucleases are engineered to cleave a specific sequence in a gene, and in other embodiments, engineered transcription factors are used to activate or repress a desired gene. In some embodiments, gene therapy reagents can also comprise specific donor molecules, for example, a transgene encoding a therapeutic protein, fragments of a transgene, nucleases or transcription factors.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g. an engineered nuclease) and a macromolecule (e.g. DNA) that are not dependent on-target sequence.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity. In the case of an RNA-guided nuclease system, the RNA guide is heterologous to the nuclease component (Cas9 or Cfp1) and both may be engineered.

A “DNA binding molecule” is a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g. the FokI domain), while in other embodiments, the DNA binding molecule is a guide RNA component of an RNA-guided nuclease (e.g. Cas9 or Cfp1).

A “DNA binding protein” (or binding domain) is a protein, or a domain within a larger protein, that binds DNA in a sequence-specific manner, for example through one or more zinc fingers or through interaction with one or more RVDs in a zinc finger protein or TALE, respectively. The term zinc finger DNA binding protein is often abbreviated as zinc finger protein or ZFP.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205, each incorporated by reference herein in its entirety.

Zinc finger and TALE DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the “repeat variable diresidue” or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

A “selected” zinc finger protein, TALE protein or CRISPR/Cas system is not found in nature whose production results primarily from an empirical process such as phage display, interaction trap, or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

“TtAgo” is a prokaryotic Argonaute protein thought to be involved in gene silencing. TtAgo is derived from the bacteria Thermus thermophilus. See, e.g. Swarts et al, ibid; G. Sheng et al., (2013) Proc. Natl. Acad. Sci. U.S.A. 111, 652). A “TtAgo system” is all the components required including e.g. guide DNAs for cleavage by a TtAgo enzyme.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In certain methods of the disclosure, one or more targeted nucleases as described herein create a double-stranded break (DSB) in the target sequence (e.g., cellular chromatin) at a predetermined site (e.g., a gene or locus of interest). The DSB mediates integration of a construct (e.g. donor) as described herein and a “donor” polynucleotide, having homology to the nucleotide sequence in the region of the break, can be introduced into the cell. The presence of the DSB has been shown to facilitate integration of the donor sequence. Optionally, the construct has homology to the nucleotide sequence in the region of the break. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional engineered nucleases can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Pat. Nos. 7,888,121; 7,914,796; 8,034,598; 8,623,618 and U.S. Patent Publication No. 2011/0201055, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 100,000,000 nucleotides in length (or any integer value therebetween), preferably between about 100 and 100,000 nucleotides in length (or any integer therebetween), more preferably between about 2000 and 20,000 nucleotides in length (or any value therebetween) and even more preferable, between about 5 and 15 kb (or any value therebetween).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids, minicircles and certain viral genomes. The liver specific constructs described herein may be epiosomally maintained or, alternatively, may be stably integrated into the cell.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, ligases, deubiquitinases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein, the term “product of an exogenous nucleic acid” includes both polynucleotide and polypeptide products, for example, transcription products (polynucleotides such as RNA) and translation products (polypeptides).

A “fusion” molecule is a molecule in which two or more subunit molecules are linked, preferably covalently. The subunit molecules can be the same chemical type of molecule, or can be different chemical types of molecules. Examples of fusion molecules include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “modification” of gene expression refers to a change in the activity of a gene. Modulation of expression can include, but is not limited to, gene activation and gene repression, including by modification of the gene via binding of an exogenous molecule (e.g., engineered transcription factor). Modulation may also be achieved by modification of the gene sequence via genome editing (e.g., cleavage, alteration, inactivation, random mutation). Gene inactivation refers to any reduction in gene expression as compared to a cell that has not been modified as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

A “safe harbor” locus is a locus within the genome wherein a gene may be inserted without any deleterious effects on the host cell. Most beneficial is a safe harbor locus in which expression of the inserted gene sequence is not perturbed by any read-through expression from neighboring genes. Non-limiting examples of safe harbor loci that are targeted by nuclease(s) include CCR5, CCR5, HPRT, AAVS1, Rosa and albumin. See, e.g., U.S. Pat. Nos. 7,951,925; 8,771,985; 8,110,379; 7,951,925; U.S. Publication Nos. 20100218264; 20110265198; 20130137104; 20130122591; 20130177983; 20130177960; 20150056705 and 20150159172.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

“Eukaryotic” cells include, but are not limited to, fungal cells (such as yeast), plant cells, animal cells, mammalian cells and human cells (e.g., T-cells), including stem cells (pluripotent and multipotent).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid or protein (e.g., coding function, ability to hybridize to another nucleic acid, enzymatic activity assays) are well-known in the art.

A polynucleotide “vector” or “construct” is capable of transferring gene sequences to target cells. Typically, “vector construct,” “expression vector,” “expression construct,” “expression cassette,” and “gene transfer vector,” mean any nucleic acid construct capable of directing the expression of a gene of interest and which can transfer gene sequences to target cells. Thus, the term includes cloning, and expression vehicles, as well as integrating vectors.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the expression cassettes of the invention can be administered. Subjects of the present invention include those with a disorder or those at risk for developing a disorder.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease. An “intended” or “on-target” sequence is the sequence to which the binding molecule is intended to bind and an “unintended” or “off-target” sequence includes any sequence bound by the binding molecule that is not the intended target.

The term “lipid” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

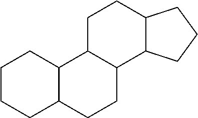 [SMILES: C1CCC2C(C1)CCC1C3CCCC3CCC21]

Non-limiting examples of steroids include cholesterol, and the like.

A “cationic lipid” refers to a lipid capable of being positively charged. Exemplary cationic lipids include one or more amine group(s) which bear the positive charge. Preferred cationic lipids are ionizable such that they can exist in a positively charged or neutral form depending on pH. The ionization of the cationic lipid affects the surface charge of the lipid nanoparticle under different pH conditions. This charge state can influence plasma protein absorption, blood clearance and tissue distribution (Semple, S. C., et al., (1998) Adv. Drug Deliv Rev 32:3-17) as well as the ability to form endosomolytic non-bilayer structures (Hafez, I. M., et al., (2001) Gene Ther 8:1188-1196) critical to the intracellular delivery of nucleic acids.

The term “lipid nanoparticle” refers to particles having at least one dimension on the order of nanometers (e.g., 1-1,000 nm) which include one or more of the compounds of formula (I), (II), (III) or (IV) or other specified cationic lipids. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the invention comprise a nucleic acid. Such lipid nanoparticles typically comprise a compound of Formula (I), (II), (III) or (IV) and one or more excipient selected from neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids, cationic lipids, pegylated lipids and their method of preparation are disclosed in, e.g., U.S. Patent Publication Nos. 2004/0142025, 2007/0042031 and PCT Pub. Nos. WO 2013/016058, WO 2013/086373, WO 2015/199952, WO 2017/004143 and WO 2017/075531, the full disclosures of which are herein incorporated by reference in their entirety for all purposes.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include compounds of Formula (IV), 1-(monomethoxy polyethyleneglycol)-2,3-dimyristoylglycerol (PEG DMG) and the like.

The term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of a number of lipid species that exist in either a positively charged or negatively charged form independent of the pH within a useful physiological range e.g. pH˜3 to pH˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. DOTAP, DOTMA), dialkyl dimethylaminopropanes, ethyl phosphocholines, dimethylaminoethane carbamoyl sterols (e.g. DC-Chol).

“Alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C1-C24 alkyl), one to twelve carbon atoms (C1-C12 alkyl), one to eight carbon atoms (C1-C8 alkyl) or one to six carbon atoms (C1-C6 alkyl) and which is attached to the rest of the molecule by a single bond, e.g., methyl, ethyl, n propyl, 1 methylethyl (iso propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group is optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless otherwise stated specifically in the specification, a cycloalkyl group is optionally substituted.

“Heterocyclyl” or “heterocyclic ring” refers to a stable 3 to 18 membered non aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1 dioxo thiomorpholinyl. Unless stated otherwise specifically in the specification, a heterocyclyl group may be optionally substituted.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (ORa, where Ra is C1-C12 alkyl or cycloalkyl); carboxyl groups (OC(═O)Ra or —C(═O)ORa, where Ra is H, C1-C12 alkyl or cycloalkyl); amine groups (NRaRb, where Ra and Rb are each independently H, C1-C12 alkyl or cycloalkyl); C1-C12 alkyl groups; and cycloalkyl groups. In some embodiments the substituent is a C1-C12 alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

Embodiments of the invention disclosed herein are also meant to encompass LNPs comprising all pharmaceutically acceptable compounds of the compound of Formula (I), (II), (III) and (IV) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labelled compounds of structure (I), (II), (III) and (IV), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I), (II), (III), (IV) and (V) can generally be prepared by conventional techniques known to those skilled in the art or by processes analogous to those described in the Preparations and Examples as set out below using an appropriate isotopically-labeled reagent in place of the non-labeled reagent previously employed.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the invention which, when administered to a mammal, preferably a human, is sufficient to effect treatment in the mammal, preferably a human. The amount of a lipid nanoparticle of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. Cancer, monogenic diseases and graft versus host disease are non-limiting examples of conditions that may be treated using the compositions and methods described herein.

The cationic lipids (e.g., compounds of Formula (I), (II), (III) or (IV)), or their pharmaceutically acceptable salts may contain one or more asymmetric centers and may thus give rise to enantiomers, diastereomers, and other stereoisomeric forms that may be defined, in terms of absolute stereochemistry, as (R) or (S) or, as (D) or (L) for amino acids. The present invention is meant to include all such possible isomers, as well as their racemic and optically pure forms. Optically active (+) and (−), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

The “wobble position” is defined as the third nucleotide in an mRNA encoding a codon which can be changed (substituted) with an alternative nucleotide without changing the identity of the resulting amino acid once translated.

DNA-Binding Molecules and Domains

Described herein are compositions comprising a DNA-binding domain that specifically binds to a target site in any gene or locus of interest. Any DNA-binding domain can be used in the compositions and methods disclosed herein, including but not limited to a zinc finger DNA-binding domain, a TALE DNA binding domain, the DNA-binding portion (sgRNA) of a CRISPR/Cas nuclease, or a DNA-binding domain from a meganuclease.

In certain embodiments, the DNA binding domain comprises a zinc finger protein. Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties. In certain embodiments, the DNA-binding domain comprises a zinc finger protein disclosed in U.S. Patent Publication No. 2012/0060230 (e.g., Table 1), incorporated by reference in its entirety herein.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in U.S. Pat. No. 6,794,136.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in U.S. Pat. No. 6,794,136.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Usually, the ZFPs of the LNPs described herein include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains.

ZFN-LNPs as described herein can include ZFNs in which the ZFP is further altered to increase its specificity for its intended target relative to other unintended cleavage sites, known as off-target sites, for example by introduction of mutations to the ZFP backbone as described in U.S. patent application Ser. No. 15/685,580. Thus, the engineered repressors and/or engineered nucleases described herein can comprise mutations in one or more of their DNA binding domain backbone regions and/or one or more mutations in their transcriptional regulatory domains. These ZFPs can include mutations to amino acids within the ZFP DNA binding domain (‘ZFP backbone’) to remove amino acid residues that can interact non-specifically with phosphates on the DNA backbone, but they do not comprise changes in the DNA recognition helices. Thus, the invention includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In preferred embodiments, mutations are made at position (−5), (−9) and/or position (−14) relative to the DNA binding helix. In some embodiments, a zinc finger may comprise one or more mutations at (−5), (−9) and/or (−14). In further embodiments, one or more zinc finger in a multi-finger zinc finger protein may comprise mutations in (−5), (−9) and/or (−14). In some embodiments, the amino acids at (−5), (−9) and/or (−14) (e.g. an arginine (R) or lysine (K)) are mutated to an alanine (A), leucine (L), Ser (S), Asp (N), Glu (E), Tyr (Y) and/or glutamine (Q). In other embodiments, the fusion polypeptides can comprise mutations in the zinc finger DNA binding domain where the amino acids at the (−5), (−9) and/or (−14) positions are changed to any of the above listed amino acids in any combination (see e.g. U.S. patent application Ser. No. 15/685,580).

In some embodiments, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128.

In other embodiments, the DNA binding domain comprises an engineered domain from a Transcriptional Activator-Like (TAL) effector (TALE) similar to those derived from the plant pathogens Xanthomonas (see Boch et al, (2009) Science 326: 1509-1512 and Moscou and Bogdanove, (2009) Science 326: 1501) and Ralstonia (see Heuer et al (2007) Applied and Environmental Microbiology 73(13): 4379-4384); U.S. Patent Application Nos. 20110301073 and 20110145940. The plant pathogenic bacteria of the genus Xanthomonas are known to cause many diseases in important crop plants. Pathogenicity of Xanthomonas depends on a conserved type III secretion (T3 S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS 1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TAL effectors depends on the sequences found in the tandem repeats. The repeated sequence comprises approximately 102 base pairs and the repeats are typically 91-100% homologous with each other (Bonas et al, ibid). Polymorphism of the repeats is usually located at positions 12 and 13 and there appears to be a one-to-one correspondence between the identity of the hypervariable diresidues (the repeat variable diresidue or RVD region) at positions 12 and 13 with the identity of the contiguous nucleotides in the TAL-effector's target sequence (see Moscou and Bogdanove, (2009) Science 326:1501 and Boch et al (2009) Science 326:1509-1512). Experimentally, the natural code for DNA recognition of these TAL-effectors has been determined such that an HD sequence at positions 12 and 13 (Repeat Variable Diresidue or RVD) leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN), including TALENs with atypical RVDs. See, e.g., U.S. Pat. No. 8,586,526.

In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224).

In still further embodiments, the nuclease comprises a compact TALEN. These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley et al (2013) Nat Comm: 1-8 DOI: 10.1038/ncomms2782). In addition, the nuclease domain may also exhibit DNA-binding functionality. Any TALENs may be used in combination with additional TALENs (e.g., one or more TALENs (cTALENs or FokI-TALENs) with one or more mega-TALEs.

In addition, as disclosed in these and other references, zinc finger domains and/or multi-fingered zinc finger proteins or TALEs may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in U.S. Pat. No. 6,794,136. In certain embodiments, the DNA-binding domain is part of a CRISPR/Cas nuclease system, including a single guide RNA (sgRNA) DNA binding molecule that binds to DNA. See, e.g., U.S. Pat. No. 8,697,359 and U.S. Patent Publication Nos. 20150056705 and 20150159172. The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

In some embodiments, the DNA binding domain is part of a TtAgo system (see Swarts et al, ibid; Sheng et al, ibid). In eukaryotes, gene silencing is mediated by the Argonaute (Ago) family of proteins. In this paradigm, Ago is bound to small (19-31 nt) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan et al., (2005) Mol. Cell 19, 405; Olovnikov, et al. (2013) Mol. Cell 51, 594; Swarts et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts et al. ibid). TtAgo associates with either 15 nt or 13-25 nt single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al., ibid). Ago from Rhodobacter sphaeroides (RsAgo) has similar properties (Olivnikov et al. ibid).

Exogenous guide DNAs of arbitrary DNA sequence can be loaded onto the TtAgo protein (Swarts et al. ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37° C. Ago-RNA-mediated DNA cleavage could be used to affect a panopoly of outcomes including gene knock-out, targeted gene addition, gene correction, targeted gene deletion using techniques standard in the art for exploitation of DNA breaks.

Thus, any DNA-binding domain can be used.

Fusion Molecules

LNPs as describe herein can include fusion molecules comprising DNA-binding domains (e.g., ZFPs or TALEs, CRISPR/Cas components such as single guide RNAs) and a heterologous regulatory (functional) domain (or functional fragment thereof) are also provided. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases) and their associated factors and modifiers. U.S. Patent Application Publication Nos. 20050064474; 20060188987 and 20070218528 for details regarding fusions of DNA-binding domains and nuclease cleavage domains, incorporated by reference in their entireties herein.

Suitable domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel et al., EMBO J. 11, 4961-4968 (1992) as well as p300, CBP, PCAF, SRC1 PvALF, AtHD2A and ERF-2. See, for example, Robyr et al. (2000) Mol. Endocrinol. 14:329-347; Collingwood et al. (1999)J. Mol. Endocrinol. 23:255-275; Leo et al. (2000) Gene 245:1-11; Manteuffel-Cymborowska (1999) Acta Biochim. Pol. 46:77-89; McKenna et al. (1999) J. SteroidBiochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

It will be clear to those of skill in the art that, in the formation of a fusion protein (or a nucleic acid encoding same) between a DNA-binding domain and a functional domain, either an activation domain or a molecule that interacts with an activation domain is suitable as a functional domain. Essentially any molecule capable of recruiting an activating complex and/or activating activity (such as, for example, histone acetylation) to the target gene is useful as an activating domain of a fusion protein. Insulator domains, localization domains, and chromatin remodeling proteins such as ISWI-containing domains and/or methyl binding domain proteins suitable for use as functional domains in fusion molecules are described, for example, in U.S. Patent Applications 2002/0115215 and 2003/0082552 and in WO 02/44376.

Exemplary repression domains include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al. (2000) Proc. Natl. Acad. Sci. USA 97:3930-3935. Furthermore, single guide RNAs of the CRISPR/Cas system associate with functional domains to form active transcriptional regulators and nucleases.

In certain embodiments, the target site is present in an accessible region of cellular chromatin. Accessible regions can be determined as described, for example, in U.S. Pat. Nos. 7,217,509 and 7,923,542. If the target site is not present in an accessible region of cellular chromatin, one or more accessible regions can be generated as described in U.S. Pat. Nos. 7,785,792 and 8,071,370. In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3) (Cordingley et al. (1987) Cell 48:261-270; Pina et al. (1990) Cell 60:719-731; and Cirillo et al. (1998) EMBO J. 17:244-254).

The fusion molecule may be formulated with a pharmaceutically acceptable carrier, as is known to those of skill in the art. See, for example, Remington's Pharmaceutical Sciences, 17th ed., 1985; and U.S. Pat. Nos. 6,453,242 and 6,534,261.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

Additional exemplary functional domains are disclosed, for example, in U.S. Pat. Nos. 6,534,261 and 6,933,113.

Functional domains that are regulated by exogenous small molecules or ligands may also be selected for use in the LNPs described herein. For example, RheoSwitch® technology may be employed wherein a functional domain only assumes its active conformation in the presence of the external RheoChem™ ligand (see for example US 20090136465). Thus, the ZFP may be operably linked to the regulatable functional domain wherein the resultant activity of the ZFP-TF is controlled by the external ligand.

Nucleases

In certain embodiments, the fusion protein comprises a DNA-binding binding domain and cleavage (nuclease) domain. As such, gene modification can be achieved using a nuclease, for example an engineered nuclease. Engineered nuclease technology is based on the engineering of naturally occurring DNA-binding proteins. For example, engineering of homing endonucleases with tailored DNA-binding specificities has been described. Chames et al. (2005) Nucleic Acids Res 33(20):e178; Arnould et al. (2006) J. Mol. Biol. 355:443-458. In addition, engineering of ZFPs has also been described. See, e.g., U.S. Pat. Nos. 6,534,261; 6,607,882; 6,824,978; 6,979,539; 6,933,113; 7,163,824; and 7,013,219.

In addition, ZFPs and/or TALEs have been fused to nuclease domains to create ZFNs and TALENs—a functional entity that is able to recognize its intended nucleic acid target through its engineered (ZFP or TALE) DNA binding domain and cause the DNA to be cut near the DNA binding site via the nuclease activity. See, e.g., Kim et al. (1996) Proc Nat'l Acad Sci USA 93(3):1156-1160. More recently, such nucleases have been used for genome modification in a variety of organisms. See, for example, United States Patent Publications 20030232410; 20050208489; 20050026157; 20050064474; 20060188987; 20060063231; and International Publication WO 07/014275.

Thus, the methods and compositions described herein are broadly applicable and may involve any nuclease of interest. Non-limiting examples of nucleases include meganucleases, TALENs and zinc finger nucleases. The nuclease may comprise heterologous DNA-binding and cleavage domains (e.g., zinc finger nucleases; meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site).

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site specific interaction with the target sequence of the user's choosing have been published (see U.S. Pat. No. 8,586,526). In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224). In addition, the nuclease domain may also exhibit DNA-binding functionality.

In still further embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley et al (2013) Nat Comm: 1-8 DOI: 10.1038/ncomms2782). Any TALENs may be used in combination with additional TALENs (e.g., one or more TALENs (cTALENs or FokI-TALENs) with one or more mega-TALs) or other DNA cleavage enzymes.

In certain embodiments, the nuclease comprises a meganuclease (homing endonuclease) or a portion thereof that exhibits cleavage activity. Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

DNA-binding domains from naturally-occurring meganucleases, primarily from the LAGLIDADG family, have been used to promote site-specific genome modification in plants, yeast, Drosophila, mammalian cells and mice, but this approach has been limited to the modification of either homologous genes that conserve the meganuclease recognition sequence (Monet et al. (1999), Biochem. Biophysics. Res. Common. 255: 88-93) or to pre-engineered genomes into which a recognition sequence has been introduced (Route et al. (1994), Mol. Cell. Biol. 14: 8096-106; Chilton et al. (2003), Plant Physiology. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622). Accordingly, attempts have been made to engineer meganucleases to exhibit novel binding specificity at medically or biotechnologically relevant sites (Porteus et al. (2005), Nat. Biotechnol. 23: 967-73; Sussman et al. (2004), J. Mol. Biol. 342: 31-41; Epinat et al. (2003), Nucleic Acids Res. 31: 2952-62; Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication Nos. 20070117128; 20060206949; 20060153826; 20060078552; and 20040002092). In addition, naturally-occurring or engineered DNA-binding domains from meganucleases can be operably linked with a cleavage domain from a heterologous nuclease (e.g., FokI) and/or cleavage domains from meganucleases can be operably linked with a heterologous DNA-binding domain (e.g., ZFP or TALE).

In other embodiments, the nuclease is a zinc finger nuclease (ZFN) or TALE DNA binding domain-nuclease fusion (TALEN). ZFNs and TALENs comprise a DNA binding domain (zinc finger protein or TALE DNA binding domain) that has been engineered to bind to a target site in a gene of choice and cleavage domain or a cleavage half-domain (e.g., from a restriction and/or meganuclease as described herein).

As described in detail above, zinc finger binding domains and TALE DNA binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain or TALE protein can have a novel binding specificity, compared to a naturally-occurring protein. Engineering methods include, but are not limited to, rational design and various types of selection. Rational design includes, for example, using databases comprising triplet (or quadruplet) nucleotide sequences and individual zinc finger or TALE amino acid sequences, in which each triplet or quadruplet nucleotide sequence is associated with one or more amino acid sequences of zinc fingers or TALE repeat units which bind the particular triplet or quadruplet sequence. See, for example, U.S. Pat. Nos. 6,453,242 and 6,534,261, incorporated by reference herein in their entireties.

Selection of target sites; and methods for design and construction of fusion proteins (and polynucleotides encoding same) are known to those of skill in the art and described in detail in U.S. Pat. Nos. 7,888,121 and 8,409,861, incorporated by reference in their entireties herein.

In addition, as disclosed in these and other references, zinc finger domains, TALEs and/or multi-fingered zinc finger proteins may be linked together using any suitable linker sequences, including for example, linkers of 5 or more amino acids in length (e.g., TGEKP (SEQ ID NO:41), TGGQRP (SEQ ID NO:42), TGQKP (SEQ ID NO:43), and/or TGSQKP (SEQ ID NO:44). See, e.g., U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. See, also, U.S. Pat. No. 8,772,453.

Thus, nucleases such as ZFNs, TALENs and/or meganucleases can comprise any DNA-binding domain and any nuclease (cleavage) domain (cleavage domain, cleavage half-domain). As noted above, the cleavage domain may be heterologous to the DNA-binding domain, for example a zinc finger or TAL-effector DNA-binding domain and a cleavage domain from a nuclease or a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites, but may lie 1 or more kilobases away from the cleavage site, including between 1-50 base pairs (or any value therebetween), 1-100 base pairs (or any value therebetween), 100-500 base pairs (or any value therebetween), 500 to 1000 base pairs (or any value therebetween) or even more than 1 kb from the cleavage site.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises a FokI cleavage domain used to generate the crystal structures 1FOK.pdb and 2FOK.pdb (see Wah et al (1997) Nature 388:97-100) having the sequence shown below:

Wild type FokI cleavage half domain
(SEQ ID NO: 40)
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAD
EMQRYVEENQTRNKHINPNEWWKVYPSSVTEFKFLFVSGHFKGNYKAQLT
RLNHITNCNGAVLSVEELLIGGEMIKAGTLTLEEVRRKFNNGEINF

Cleavage half domains derived from FokI may comprise a mutation in one or more of amino acid residues as shown in SEQ ID NO:40. Mutations include substitutions (of a wild-type amino acid residue for a different residue, insertions (of one or more amino acid residues) and/or deletions (of one or more amino acid residues). In certain embodiments, one or more of residues 414-426, 443-450, 467-488, 501-502, and/or 521-531 (numbered relative to SEQ ID NO:40) are mutated since these residues are located close to the DNA backbone in a molecular model of a ZFN bound to its target site described in Miller et al. ((2007) Nat Biotechnol 25:778-784). In certain embodiments, one or more residues at positions 416, 422, 447, 448, and/or 525 are mutated. In certain embodiments, the mutation comprises a substitution of a wild-type residue with a different residue, for example a serine (S) residue. See, e.g., U.S. application Ser. No. 15/685,580.

In certain embodiments, the cleavage domain comprises one or more engineered cleavage half-domain (also referred to as dimerization domain mutants) that minimize or prevent homodimerization, as described, for example, in U.S. Pat. Nos. 7,914,796; 8,034,598 and 8,623,618; and U.S. Patent Publication No. 20110201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I (numbered relative to SEQ ID NO:40) are all targets for influencing dimerization of the Fok I cleavage half-domains. The mutations may include mutations to residues found in natural restriction enzymes homologous to FokI. In a preferred embodiment, the mutation at positions 416, 422, 447, 448 and/or 525 (numbered relative to SEQ ID NO:40) comprise replacement of a positively charged amino acid with an uncharged or a negatively charged amino acid. In another embodiment, the engineered cleavage half domain comprises mutations in amino acid residues 499, 496 and 486 in addition to the mutations in one or more amino acid residues 416, 422, 447, 448, or 525, all numbered relative to SEQ ID NO:40.

In certain embodiments, the compositions described herein include engineered cleavage half-domains of Fok I that form obligate heterodimers as described, for example, in U.S. Pat. Nos. 7,914,796; 8,034,598; 8,961,281 and 8,623,618; U.S. Patent Publication Nos. 20080131962 and 20120040398. Thus, in one preferred embodiment, the invention provides fusion proteins wherein the engineered cleavage half-domain comprises a polypeptide in which the wild-type Gln (Q) residue at position 486 is replaced with a Glu (E) residue, the wild-type Ile (I) residue at position 499 is replaced with a Leu (L) residue and the wild-type Asn (N) residue at position 496 is replaced with an Asp (D) or a Glu (E) residue (“ELD” or “ELE”) in addition to one or more mutations at positions 416, 422, 447, 448, or 525 (numbered relative to SEQ ID NO: 1). In another embodiment, the engineered cleavage half domains are derived from a wild-type FokI cleavage half domain and comprise mutations in the amino acid residues 490, 538 and 537, numbered relative to wild-type FokI (SEQ ID NO: 1) in addition to the one or more mutations at amino acid residues 416, 422, 447, 448, or 525. In a preferred embodiment, the invention provides a fusion protein, wherein the engineered cleavage half-domain comprises a polypeptide in which the wild-type Glu (E) residue at position 490 is replaced with a Lys (K) residue, the wild-type Ile (I) residue at position 538 is replaced with a Lys (K) residue, and the wild-type His (H) residue at position 537 is replaced with a Lys (K) residue or an Arg (R) residue (“KKK” or “KKR”) (see U.S. Pat. No. 8,962,281, incorporated by reference herein) in addition to one or more mutations at positions 416, 422, 447, 448, or 525. See, e.g., U.S. Pat. Nos. 7,914,796; 8,034,598 and 8,623,618, the disclosures of which are incorporated by reference in its entirety for all purposes. In other embodiments, the engineered cleavage half domain comprises the “Sharkey” and/or some of the “Sharkey” mutations (see Guo et al, (2010) J. Mol. Biol. 400(1):96-107).

In another embodiment, the engineered cleavage half domains are derived from a wild-type FokI cleavage half domain and comprise mutations in the amino acid residues 490, and 538, numbered relative to wild-type FokI in addition to the one or more mutations at amino acid residues 416, 422, 447, 448, or 525. In a preferred embodiment, the invention provides a fusion protein, wherein the engineered cleavage half-domain comprises a polypeptide in which the wild-type Glu (E) residue at position 490 is replaced with a Lys (K) residue, and the wild-type Ile (I) residue at position 538 is replaced with a Lys (K) residue (“KK”) in addition to one or more mutations at positions 416, 422, 447, 448, or 525. In a preferred embodiment, the invention provides a fusion protein, wherein the engineered cleavage half-domain comprises a polypeptide in which the wild-type Gln (Q) residue at position 486 is replaced with an Glu (E) residue, and the wild-type Ile (I) residue at position 499 is replaced with a Leu (L) residue (“EL”) (See U.S. Pat. No. 8,034,598, incorporated by reference herein) in addition to one or more mutations at positions 416, 422, 447, 448, or 525.

In one aspect, the invention provides a fusion protein wherein the engineered cleavage half-domain comprises a polypeptide in which the wild-type amino acid residue at one or more of positions 387, 393, 394, 398, 400, 402, 416, 422, 427, 434, 439, 441, 447, 448, 469, 487, 495, 497, 506, 516, 525, 529, 534, 559, 569, 570, 571 in the FokI catalytic domain are mutated. In some embodiments, these mutations in the FokI domain prevent or lessen non-specific interactions between the FokI domain and the phosphate contained in a DNA backbone. In some embodiments, the one or more mutations alter the wild type amino acid from a positively charged residue to a neutral residue or a negatively charged residue. In any of these embodiments, the mutants described may also be made in a FokI domain comprising one or more additional mutations. In preferred embodiments, these additional mutations are in the dimerization domain, e.g. at positions 499, 496, 486, 490, 538 and 537.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases (e.g., ZFNs and/or TALENs) can be screened for activity prior to use, for example in a yeast-based chromosomal system as described in as described in U.S. Pat. No. 8,563,314.

In certain embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al, (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “Cas” includes both Cas9 and Cfp1 proteins. Thus, as used herein, a “CRISPR/Cas system” refers both CRISPR/Cas and/or CRISPR/Cfp1 systems, including both nuclease and/or transcription factor systems.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof such as derivative Cas proteins. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein. In some embodiments, the Cas protein is a small Cas9 ortholog for delivery via an AAV vector (Ran et al (2015) Nature 510, p. 186).

The nuclease(s) may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266; 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffery et al. (2016) Nucleic Acids Res. 44(2):e11. doi: 10.1093/nar/gkv878. Epub 2015 Oct. 19.

### Compounds

In an aspect, the invention includes LNPs comprising cationic lipid compounds which are capable of combining with other lipid components such as neutral lipids, charged lipids, steroids and/or polymer conjugated-lipids to form lipid nanoparticles with oligonucleotides. Without wishing to be bound by theory, it is thought that these lipid nanoparticles shield oligonucleotides from degradation in the serum and provide for effective delivery of oligonucleotides to cells in vitro and in vivo.

In one embodiment, the LNPs comprise a polynucleotide having activity as a gene therapy reagent and a lipid compound having the structure of Formula (I)

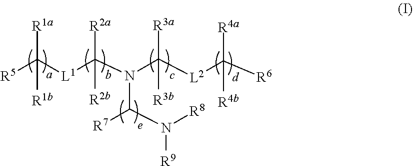

or a pharmaceutically acceptable salt, tautomer, prodrug or stereoisomer thereof, wherein:

L¹ and L² are each independently —O(C═O)—, (C═O)O— or a carbon-carbon double bond;

R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently methyl or cycloalkyl;

R⁷ is, at each occurrence, independently H or C₁-C₁₂ alkyl;

R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

a and d are each independently an integer from 0 to 24;

b and c are each independently an integer from 1 to 24; and

e is 1 or 2.

In certain embodiments of the Formula (I) compound at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments, at least one of R^{1a}, R^{2a}, R^{3a} or R^{4a} is C₁-C₁₂ alkyl, or at least one of L¹ or L² is —O(C═O)— or —(C═O)O—; and R^{1a} and R^{1b} are not isopropyl when a is 6 or n-butyl when a is 8.

In the compound of Formula I, any one of L¹ or L² may be —O(C═O)— or a carbon-carbon double bond. L¹ and L² may each be —O(C═O)— or may each be a carbon-carbon double bond.

In some embodiments of Formula I, one of L¹ or L² is —O(C═O)—. In other embodiments of Formula I, both L¹ and L² are —O(C═O)—.

In some embodiments of Formula I, one of L¹ or L² is —(C═O)O—. In other embodiments of Formula I, both L¹ and L² are —(C═O)O—.

In some embodiments of Formula I, one of L¹ or L² is a carbon-carbon double bond. In other embodiments of Formula I, both L¹ and L² are a carbon-carbon double bond.

In still other embodiments of Formula I, one of L¹ or L² is —O(C═O)— and the other of L¹ or L² is —(C═O)O—. In more embodiments of Formula I, one of L¹ or L² is —O(C═O)— and the other of L¹ or L² is a carbon-carbon double bond. In yet more embodiments of Formula I, one of L¹ or L² is —(C═O)O— and the other of L¹ or L² is a carbon-carbon double bond.

It is understood that “carbon-carbon” double bond, as used throughout this disclosure, refers to one of the following structures:

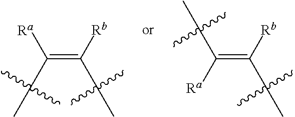

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments of Formula I, the lipid compounds have the following structure (Ia):

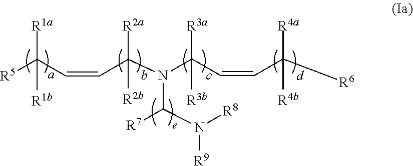

In other embodiments of Formula I, the lipid compounds have the following structure (Ib):

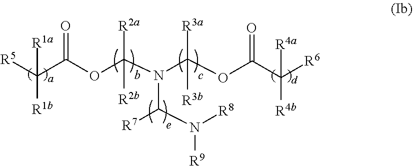

In yet other embodiments of Formula I, the lipid compounds have the following structure (Ic):

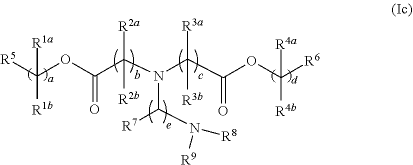

In certain embodiments of Formula I, a, b, c and d are each independently an integer from 2 to 12 or an integer from 4 to 12. In other embodiments of Formula I, a, b, c and d are each independently an integer from 8 to 12 or 5 to 9. In some certain embodiments of Formula I, a is 0. In some embodiments of Formula I, a is 1. In other embodiments of Formula I, a is 2. In more embodiments of Formula I, a is 3. In yet other embodiments of Formula I, a is 4. In some embodiments of Formula I, a is 5. In other embodiments of Formula I, a is 6. In more embodiments of Formula I, a is 7. In yet other embodiments of Formula I, a is 8. In some embodiments of Formula I, a is 9. In other embodiments of Formula I, a is 10. In more embodiments of Formula I, a is 11. In yet other embodiments of Formula I, a is 12. In some embodiments of Formula I, a is 13. In other embodiments of Formula I, a is 14. In more embodiments of Formula I, a is 15. In yet other embodiments of Formula I, a is 16.

In some embodiments of Formula I, b is 1. In other embodiments of Formula I, b is 2. In more embodiments of Formula I, b is 3. In yet other embodiments of Formula I, b is 4. In some embodiments of Formula I, b is 5. In other embodiments of Formula I, b is 6. In more embodiments of Formula I, b is 7. In yet other embodiments of Formula I, b is 8. In some embodiments of Formula I, b is 9. In other embodiments of Formula I, b is 10. In more embodiments of Formula I, b is 11. In yet other embodiments of Formula I, b is 12. In some embodiments of Formula I, b is 13. In other embodiments of Formula I, b is 14. In more embodiments of Formula I, b is 15. In yet other embodiments of Formula I, b is 16.

In some embodiments of Formula I, c is 1. In other embodiments of Formula I, c is 2. In more embodiments of Formula I, c is 3. In yet other embodiments of Formula I, c is 4. In some embodiments of Formula I, c is 5. In other embodiments of Formula I, c is 6. In more embodiments of Formula I, c is 7. In yet other embodiments of Formula I, c is 8. In some embodiments of Formula I, c is 9. In other embodiments of Formula I, c is 10. In more embodiments of Formula I, c is 11. In yet other embodiments of Formula I, c is 12. In some embodiments of Formula I, c is 13. In other embodiments of Formula I, c is 14. In more embodiments of Formula I, c is 15. In yet other embodiments of Formula I, c is 16.

In some certain embodiments of Formula I, d is 0. In some embodiments of Formula I, d is 1. In other embodiments of Formula I, d is 2. In more embodiments of Formula I, d is 3. In yet other embodiments of Formula I, d is 4. In some embodiments of Formula I, d is 5. In other embodiments of Formula I, d is 6. In more embodiments of Formula I, d is 7. In yet other embodiments of Formula I, d is 8. In some embodiments of Formula I, d is 9. In other embodiments of Formula I, d is 10. In more embodiments of Formula I, d is 11. In yet other embodiments of Formula I, d is 12. In some embodiments of Formula I, d is 13. In other embodiments of Formula I, d is 14. In more embodiments of Formula I, d is 15. In yet other embodiments of Formula I, d is 16.

In some other various embodiments of Formula I, a and d are the same. In some other embodiments of Formula I, b and c are the same. In some other specific embodiments of Formula I a and d are the same and b and c are the same.

The sum of a and b and the sum of c and d are factors which may be varied to obtain a lipid having the desired properties. In one embodiment of Formula I, a and b are chosen such that their sum is an integer ranging from 14 to 24. In other embodiments of Formula I, c and d are chosen such that their sum is an integer ranging from 14 to 24. In further embodiment of Formula I, the sum of a and b and the sum of c and d are the same. For example, in some embodiments of Formula I the sum of a and b and the sum of c and d are both the same integer which may range from 14 to 24. In still more embodiments of Formula I, a. b, c and d are selected such the sum of a and b and the sum of c and d is 12 or greater.

In some embodiments of Formula I, e is 1. In other embodiments of Formula I, e is 2.

The substituents at R^{1a}, R^{2a}, R^{3a} and R^{4a} are not particularly limited. In certain embodiments of Formula I R^{1a}, R^{2a}, R^{3a} and R^{4a} are H at each occurrence. In certain other embodiments of Formula I at least one of R^{1a}, R^{2a}, R^{3a} and R^{4a} is C₁-C₁₂ alkyl. In certain other embodiments of Formula I at least one of R^{1a}, R^{2a}, R^{3a} and R^{4a} is C₁-C₈ alkyl. In certain other embodiments of Formula I at least one of R^{1a}, R^{2a}, R^{3a} and R^{4a} is C₁-C₆ alkyl. In some of the foregoing embodiments of Formula I, the C₁-C₈ alkyl is methyl, ethyl, n-propyl, iso-propyl, n-butyl, iso-butyl, tert-butyl, n-hexyl or n-octyl.

In certain embodiments of Formula I, R^{1a}, R^{1b}, R^{4a} and R^{4b} are C₁-C₁₂ alkyl at each occurrence.

In further embodiments of Formula I, at least one of R^{1b}, R^{2b}, R^{3b} and R^{4b} is H or R^{1b}, R^{2b}, R^{3b} and R^{4b} are H at each occurrence.

In certain embodiments of Formula I, R^{1b} together with the carbon atom to which it is bound is taken together with an adjacent R^{1b} and the carbon atom to which it is bound to form a carbon-carbon double bond. In other embodiments of Formula I R^{4b} together with the carbon atom to which it is bound is taken together with an adjacent R^{4b} and the carbon atom to which it is bound to form a carbon-carbon double bond.

The substituents at R⁵ and R⁶ are not particularly limited in the foregoing embodiments. In certain embodiments of Formula I one or both of R⁵ or R⁶ is methyl. In certain other embodiments of Formula I one or both of R⁵ or R⁶ is cycloalkyl for example cyclohexyl. In these embodiments the cycloalkyl may be substituted or not substituted. In certain other embodiments the cycloalkyl is substituted with C₁-C₁₂ alkyl, for example tert-butyl.

The substituents at R⁷ are not particularly limited in the foregoing embodiments. In certain embodiments of Formula I at least one R⁷ is H. In some other embodiments, R⁷ is H at each occurrence. In certain other embodiments of Formula I R⁷ is C₁-C₁₂ alkyl.

In certain other embodiments of Formula I, one of R⁸ or R⁹ is methyl. In other embodiments, both R⁸ and R⁹ are methyl.

In some different embodiments of Formula I, R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, the LNP comprises a compound having one of the structures set forth in Table I below.

TABLE I
Representative Lipid Compounds of Formula (I)

| | | Prep. |
|---|---|---|
| No. | Structure | Method* |
| I-1 | 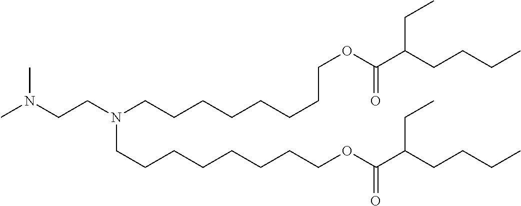 [SMILES: CCCCC(CC)C(=O)OCCCCCCCCN(CCCCCCCCOC(=O)C(CC)CCCC)CCN(C)C] | B |
| I-2 | 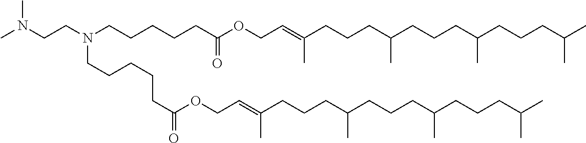 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | A |
| I-3 | 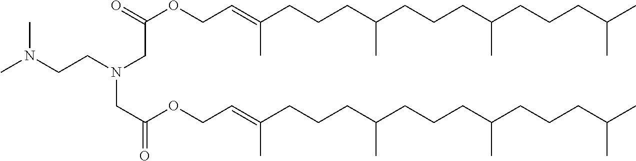 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | A |
| I-4 | 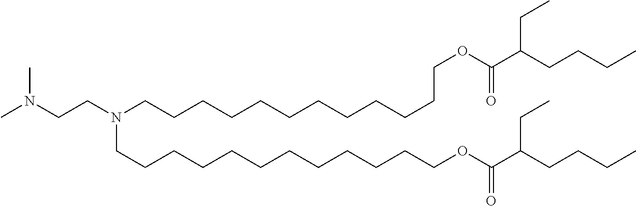 [SMILES: CCCCC(CC)C(=O)OCCCCCCCCCCCCN(CCCCCCCCCCCCOC(=O)C(CC)CCCC)CCN(C)C] | B |
| I-5 | 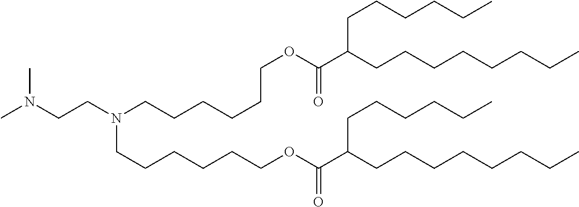 [SMILES: CCCCCCCCC(CCCCCC)C(=O)OCCCCCCN(CCCCCCOC(=O)C(CCCCCC)CCCCCCCC)CCN(C)C] | B |
| I-6 | 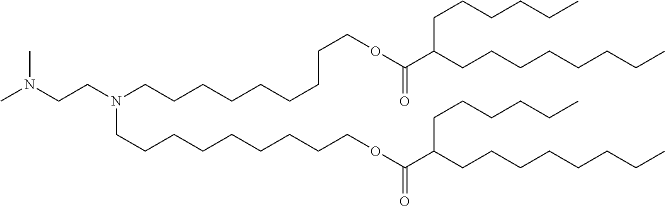 [SMILES: CCCCCCCCC(CCCCCC)C(=O)OCCCCCCCCCN(CCCCCCCCCOC(=O)C(CCCCCC)CCCCCCCC)CCN(C)C] | B |
| I-7 | 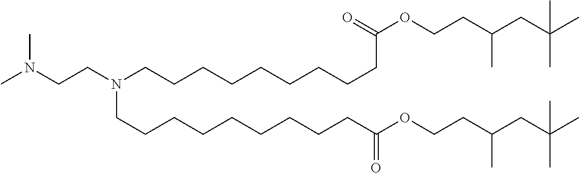 [SMILES: CC(CCOC(=O)CCCCCCCCCN(CCCCCCCCCC(=O)OCCC(C)CC(C)(C)C)CCN(C)C)CC(C)(C)C] | A |
| I-8 | 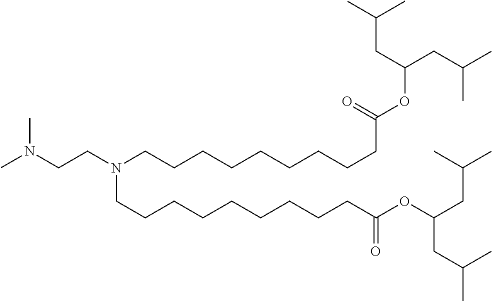 [SMILES: CC(C)CC(CC(C)C)OC(=O)CCCCCCCCCN(CCCCCCCCCC(=O)OC(CC(C)C)CC(C)C)CCN(C)C] | A |
| I-9 | 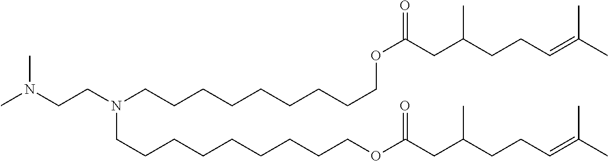 [SMILES: CC(C)=CCCC(C)CC(=O)OCCCCCCCCCN(CCCCCCCCCOC(=O)CC(C)CCC=C(C)C)CCN(C)C] | B |
| I-10 | 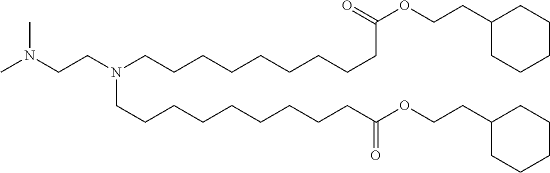 [SMILES: CN(C)CCN(CCCCCCCCCC(=O)OCCC1CCCCC1)CCCCCCCCCC(=O)OCCC1CCCCC1] | A |
| I-11 | 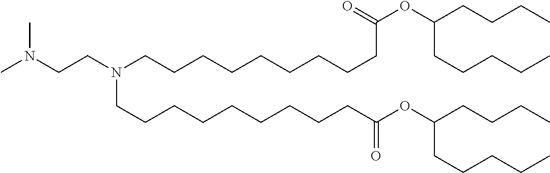 [SMILES: CCCCCC(CCCC)OC(=O)CCCCCCCCCN(CCCCCCCCCC(=O)OC(CCCC)CCCCC)CCN(C)C] | A |
| I-12 | 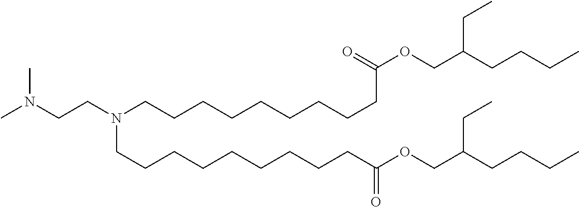 [SMILES: CCCCC(CC)COC(=O)CCCCCCCCCN(CCCCCCCCCC(=O)OCC(CC)CCCC)CCN(C)C] | A |
| I-13 | 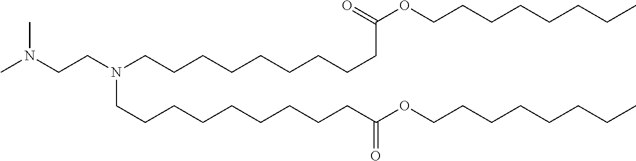 [SMILES: CCCCCCCCOC(=O)CCCCCCCCCN(CCCCCCCCCC(=O)OCCCCCCCC)CCN(C)C] | A |
| I-14 | 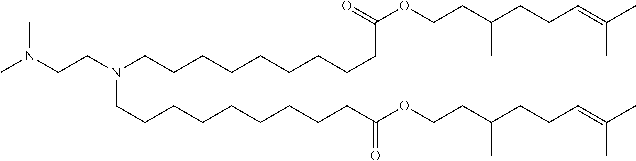 [SMILES: CC(C)=CCCC(C)CCOC(=O)CCCCCCCCCN(CCCCCCCCCC(=O)OCCC(C)CCC=C(C)C)CCN(C)C] | A |
| I-15 | 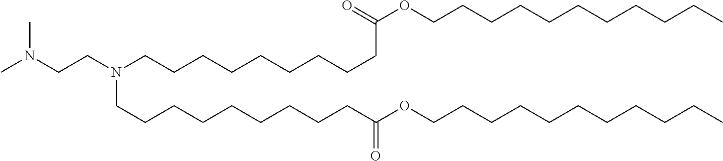 [SMILES: CCCCCCCCCCCOC(=O)CCCCCCCCCN(CCCCCCCCCC(=O)OCCCCCCCCCCC)CCN(C)C] | A |
| I-16 | 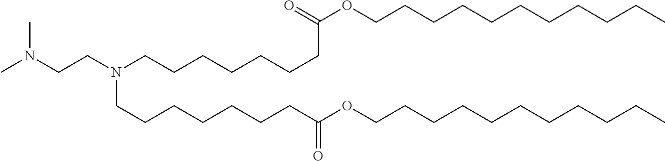 [SMILES: CCCCCCCCCCCOC(=O)CCCCCCCN(CCCCCCCC(=O)OCCCCCCCCCCC)CCN(C)C] | A |
| I-17 | 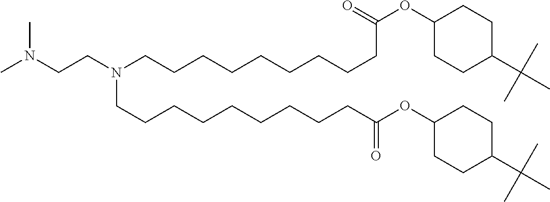 [SMILES: CN(C)CCN(CCCCCCCCCC(=O)OC1CCC(C(C)(C)C)CC1)CCCCCCCCCC(=O)OC1CCC(C(C)(C)C)CC1] | A |
| I-18 | 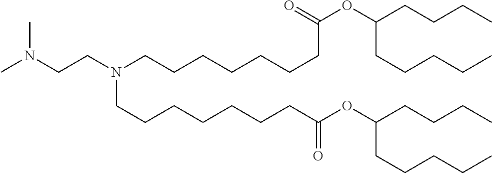 [SMILES: CCCCCC(CCCC)OC(=O)CCCCCCCN(CCCCCCCC(=O)OC(CCCC)CCCCC)CCN(C)C] | A |
| I-19 | 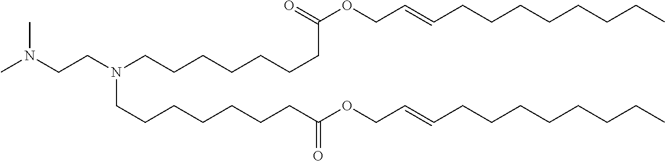 [SMILES: CCCCCCCC/C=C/COC(=O)CCCCCCCN(CCCCCCCC(=O)OC/C=C/CCCCCCCC)CCN(C)C] | A |
| I-20 | 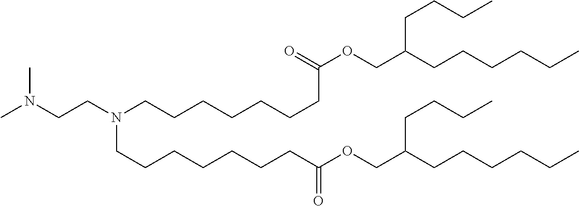 [SMILES: CCCCCCC(CCCC)COC(=O)CCCCCCCN(CCCCCCCC(=O)OCC(CCCC)CCCCCC)CCN(C)C] | A |
| I-21 | 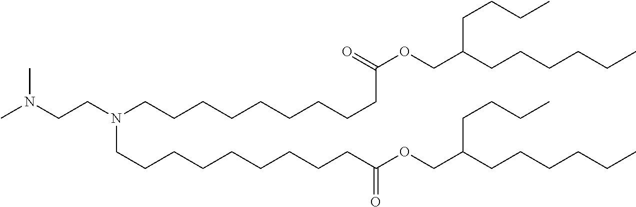 [SMILES: CCCCCCC(CCCC)COC(=O)CCCCCCCCCN(CCCCCCCCCC(=O)OCC(CCCC)CCCCCC)CCN(C)C] | A |
| I-22 | 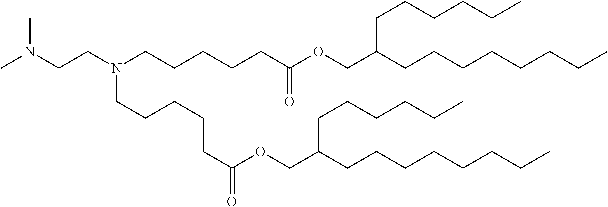 [SMILES: CCCCCCCCC(CCCCCC)COC(=O)CCCCCN(CCCCCC(=O)OCC(CCCCCC)CCCCCCCC)CCN(C)C] | A |
| I-23 | 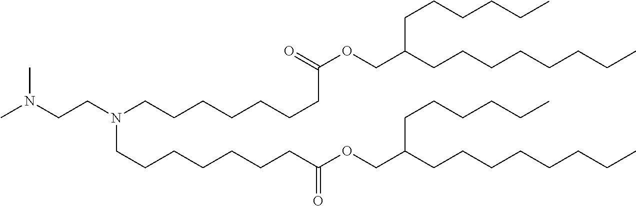 [SMILES: CCCCCCCCC(CCCCCC)COC(=O)CCCCCCCN(CCCCCCCC(=O)OCC(CCCCCC)CCCCCCCC)CCN(C)C] | A |
| I-24 | 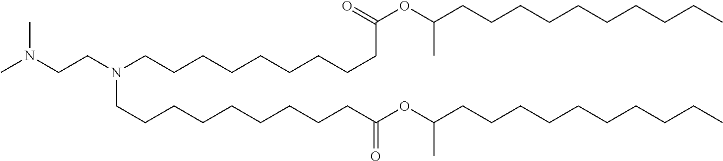 [SMILES: CCCCCCCCCCC(C)OC(=O)CCCCCCCCCN(CCCCCCCCCC(=O)OC(C)CCCCCCCCCC)CCN(C)C] | A |
| I-25 | 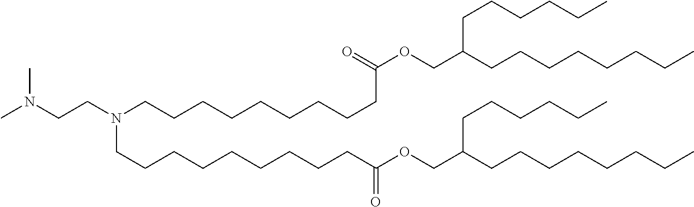 [SMILES: CCCCCCCCC(CCCCCC)COC(=O)CCCCCCCCCN(CCCCCCCCCC(=O)OCC(CCCCCC)CCCCCCCC)CCN(C)C] | A |
| I-26 | 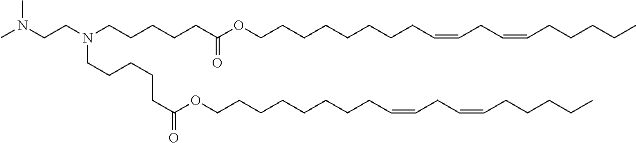 [SMILES: CCCCC/C=C\C/C=C\CCCCCCCCOC(=O)CCCCCN(CCCCCC(=O)OCCCCCCCC/C=C\C/C=C\CCCCC)CCN(C)C] | A |
| I-27 | 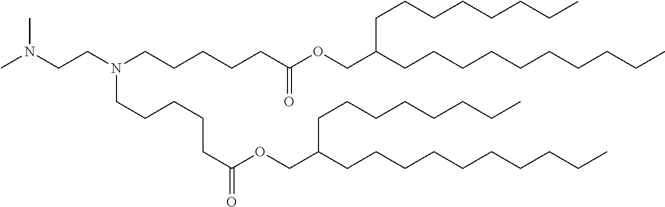 [SMILES: CCCCCCCCCCC(CCCCCCCC)COC(=O)CCCCCN(CCCCCC(=O)OCC(CCCCCCCC)CCCCCCCCCC)CCN(C)C] | A |
| I-28 | 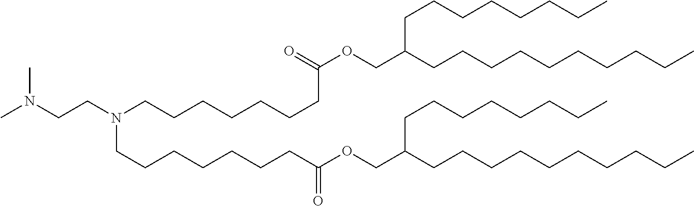 [SMILES: CCCCCCCCCCC(CCCCCCCC)COC(=O)CCCCCCCN(CCCCCCCC(=O)OCC(CCCCCCCC)CCCCCCCCCC)CCN(C)C] | A |
| I-29 | 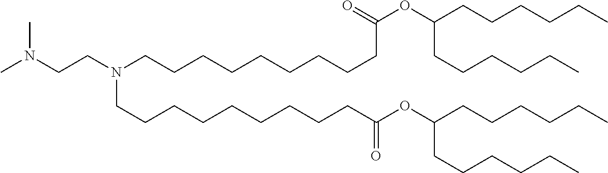 [SMILES: CCCCCCC(CCCCCC)OC(=O)CCCCCCCCCN(CCCCCCCCCC(=O)OC(CCCCCC)CCCCCC)CCN(C)C] | A |
| I-30 | 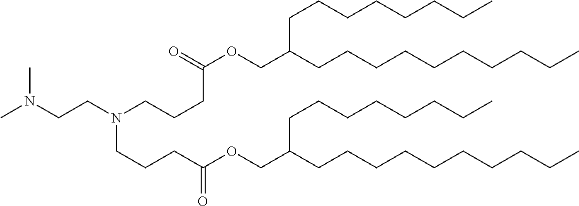 [SMILES: CCCCCCCCCCC(CCCCCCCC)COC(=O)CCCN(CCCC(=O)OCC(CCCCCCCC)CCCCCCCCCC)CCN(C)C] | A |
| I-31 | 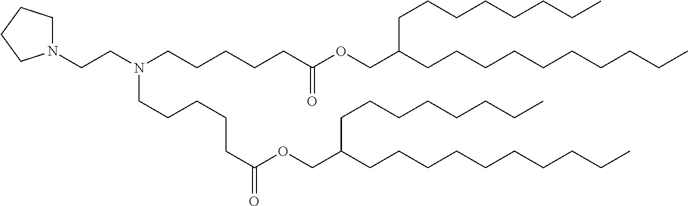 [SMILES: CCCCCCCCCCC(CCCCCCCC)COC(=O)CCCCCN(CCCCCC(=O)OCC(CCCCCCCC)CCCCCCCCCC)CCN1CCCC1] | C |
| I-32 | 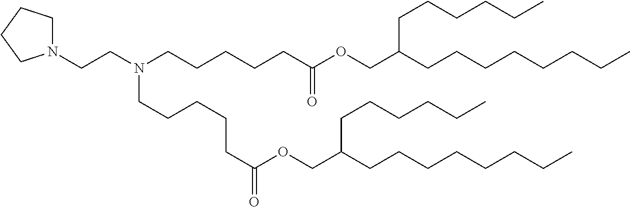 [SMILES: CCCCCCCCC(CCCCCC)COC(=O)CCCCCN(CCCCCC(=O)OCC(CCCCCC)CCCCCCCC)CCN1CCCC1] | C |
| I-33 | 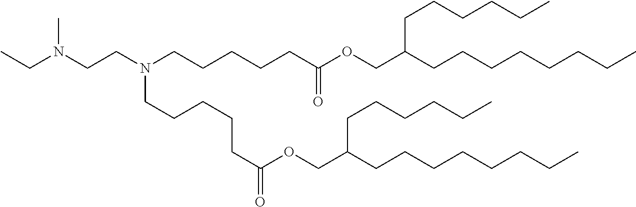 [SMILES: CCCCCCCCC(CCCCCC)COC(=O)CCCCCN(CCCCCC(=O)OCC(CCCCCC)CCCCCCCC)CCN(C)CC] | C |
| I-34 | 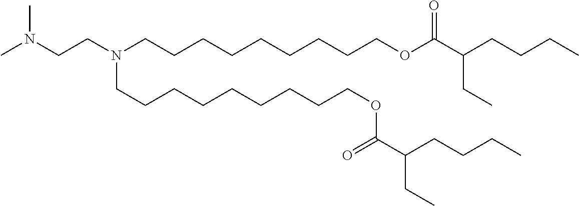 [SMILES: CCCCC(CC)C(=O)OCCCCCCCCCN(CCCCCCCCCOC(=O)C(CC)CCCC)CCN(C)C] | B |
| I-35 | 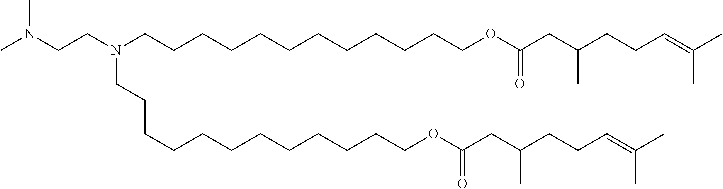 [SMILES: CC(C)=CCCC(C)CC(=O)OCCCCCCCCCCCCN(CCCCCCCCCCCCOC(=O)CC(C)CCC=C(C)C)CCN(C)C] | B |
| I-36 | 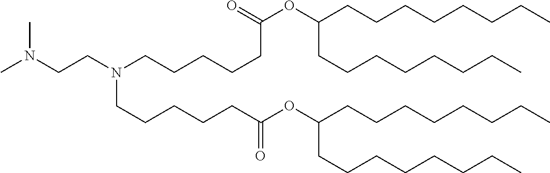 [SMILES: CCCCCCCCC(CCCCCCCC)OC(=O)CCCCCN(CCCCCC(=O)OC(CCCCCCCC)CCCCCCCC)CCN(C)C] | C |
| I-37 | 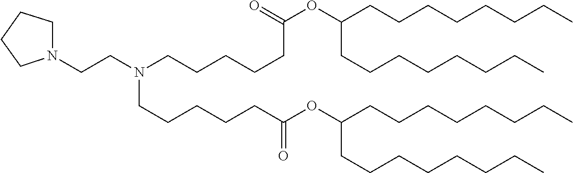 [SMILES: CCCCCCCCC(CCCCCCCC)OC(=O)CCCCCN(CCCCCC(=O)OC(CCCCCCCC)CCCCCCCC)CCN1CCCC1] | C |
| I-38 | 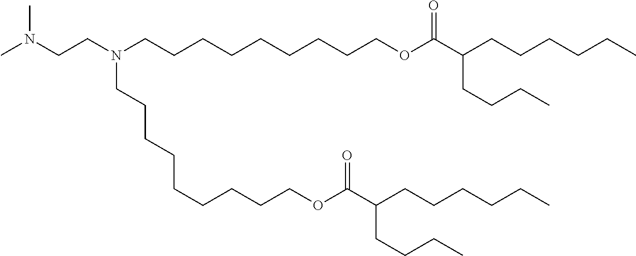 [SMILES: CCCCCCC(CCCC)C(=O)OCCCCCCCCCN(CCCCCCCCCOC(=O)C(CCCC)CCCCCC)CCN(C)C] | B |
| I-39 | 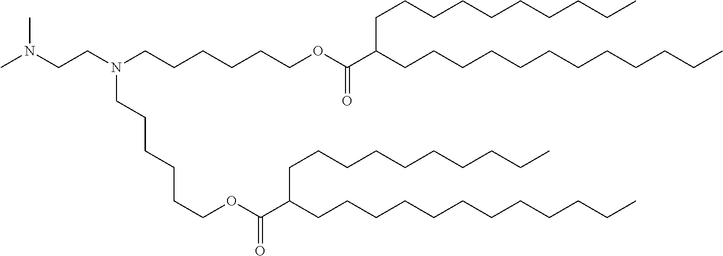 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | B |
| I-40 | 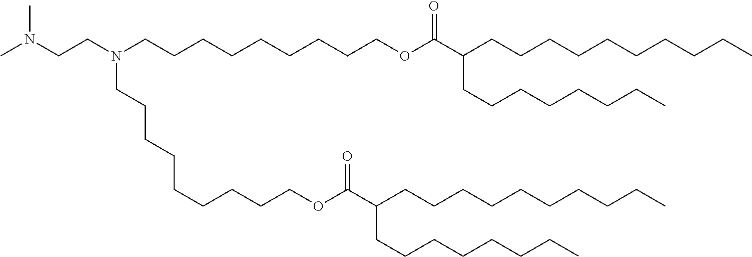 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | B |
| I-41 | 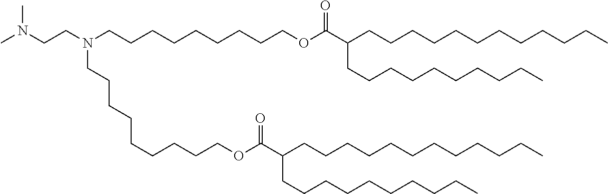 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | B |

*Refers to general synthetic method for preparation of lipid

It is understood that any embodiment of the compounds of Formula (I), as set forth above, and any specific substituent and/or variable in the compound Formula (I), as set forth above, may be independently combined with other embodiments and/or substituents and/or variables of compounds of Formula (I) to form embodiments of the inventions not specifically set forth above. In addition, in the event that a list of substituents and/or variables is listed for any particular R group, L group or variables a-e in a particular embodiment and/or claim, it is understood that each individual substituent and/or variable may be deleted from the particular embodiment and/or claim and that the remaining list of substituents and/or variables will be considered to be within the scope of the invention.

It is understood that in the present description, combinations of substituents and/or variables of the depicted formulae are permissible only if such contributions result in stable compounds.

In some embodiments, compositions comprising any one or more of the compounds of Formula (II) and a polynucleotide having activity as a gene editing/gene therapy reagent are provided. For example, in some embodiments, the compositions comprise any of the compounds of Formula (II) and a polynucleotide having activity as a gene editing/gene therapy reagent and one or more excipient selected from neutral lipids, steroids and polymer conjugated lipids. Other pharmaceutically acceptable excipients and/or carriers are also included in various embodiments of the compositions.

In some embodiments, the neutral lipid is selected from DSPC, DPPC, DMPC, DOPC, POPC, DOPE and SM. In some embodiments, the neutral lipid is DSPC. In various embodiments, the molar ratio of the compound to the neutral lipid ranges from about 2:1 to about 8:1.

In various embodiments, the compositions further comprise a steroid or steroid analogue. In certain embodiments, the steroid or steroid analogue is cholesterol. In some of these embodiments, the molar ratio of the compound to cholesterol ranges from about 2:1 to 1:1.

In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the compound to the pegylated lipid ranges from about 100:1 to about 25:1.

In some embodiments, the LNPs comprise a polynucleotide having activity as a gene therapy reagent and a lipid compound having the following Formula (II):

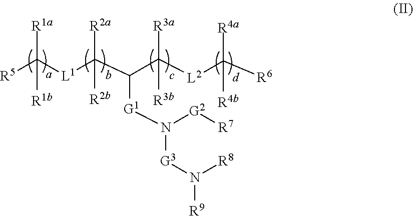

or a pharmaceutically acceptable salt, tautomer, prodrug or stereoisomer thereof, wherein:

L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;

G¹ is C₁-C₂ alkylene, —(C═O)—, —O(C═O)—, —SC(═O)—, —NR^{a}C(═O)— or a direct bond;

G² is —C(═O)—, —(C═O)O—, —C(═O)S—, —C(═O)NR^{a}— or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently H or methyl;

R⁷ is C₄-C₂₀ alkyl;

R⁸ and R⁹ are each independently C₁-C₁₂ alkyl; or R⁸ and R⁹, together with the nitrogen atom to which they are attached, form a 5, 6 or 7-membered heterocyclic ring; a, b, c and d are each independently an integer from 1 to 24; and

x is 0, 1 or 2.

In some embodiments of Formula (II), L¹ and L² are each independently-O(C═O)—, —(C═O)O— or a direct bond. In other embodiments of Formula (II), G¹ and G² are each independently —(C═O)— or a direct bond. In some different embodiments of Formula (II), L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the compound has one of the following structures (IIA) or (IIB):

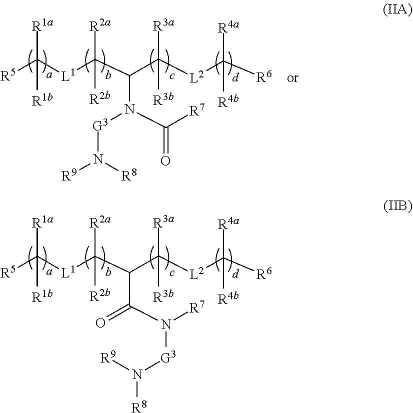

In some embodiments of Formula (II), the compound has structure (IIA). In other embodiments of Formula (II), the compound has structure (IIB).

In any of the foregoing embodiments of Formula (II), one of L¹ or L² is —O(C═O)—. For example, in some embodiments of Formula (II) each of L¹ and L² are —O(C═O)—.

In some different embodiments of Formula (II), one of L¹ or L² is —(C═O)O—. For example, in some embodiments of Formula (II) each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments of Formula (II) each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the compound has one of the following structures (IIC) or (IID):

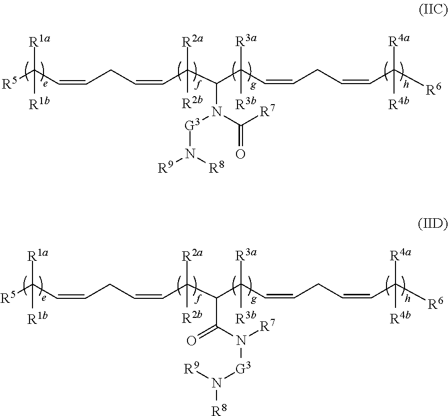

wherein e, f, g and h are each independently an integer from 1 to 12.

In some embodiments of Formula (II), the compound has structure (IIC). In other embodiments of Formula (II), the compound has structure (IID).

In various embodiments of the compounds of structures (IIC) or (IID), e, f, g and h are each independently an integer from 4 to 10.

In certain embodiments of Formula (II), a, b, c and d are each independently an integer from 2 to 12 or an integer from 4 to 12. In other embodiments of Formula (II), a, b, c and d are each independently an integer from 8 to 12 or 5 to 9. In some certain embodiments, a is 0. In some embodiments of Formula (II), a is 1. In other embodiments of Formula (II), a is 2. In more embodiments of Formula (II), a is 3. In yet other embodiments of Formula (II), a is 4. In some embodiments of Formula (II), a is 5. In other embodiments of Formula (II), a is 6. In more embodiments of Formula (II), a is 7. In yet other embodiments of Formula (II), a is 8. In some embodiments of Formula (II), a is 9. In other embodiments of Formula (II), a is 10. In more embodiments of Formula (II), a is 11. In yet other embodiments of Formula (II), a is 12. In some embodiments of Formula (II), a is 13. In other embodiments of Formula (II), a is 14. In more embodiments of Formula (II), a is 15. In yet other embodiments of Formula (II), a is 16.

In some embodiments of Formula (II), b is 1. In other embodiments of Formula (II), b is 2. In more embodiments of Formula (II), b is 3. In yet other embodiments of Formula (II), b is 4. In some embodiments of Formula (II), b is 5. In other embodiments of Formula (II), b is 6. In more embodiments of Formula (II), b is 7. In yet other embodiments of Formula (II), b is 8. In some embodiments of Formula (II), b is 9. In other embodiments of Formula (II), b is 10. In more embodiments of Formula (II), b is 11. In yet other embodiments of Formula (II), b is 12. In some embodiments of Formula (II), b is 13. In other embodiments of Formula (II), b is 14. In more embodiments of Formula (II), b is 15. In yet other embodiments of Formula (II), b is 16.

In some embodiments of Formula (II), c is 1. In other embodiments of Formula (II), c is 2. In more embodiments of Formula (II), c is 3. In yet other embodiments of Formula (II), c is 4. In some embodiments of Formula (II), c is 5. In other embodiments of Formula (II), c is 6. In more embodiments of Formula (II), c is 7. In yet other embodiments of Formula (II), c is 8. In some embodiments of Formula (II), c is 9. In other embodiments of Formula (II), c is 10. In more embodiments of Formula (II), c is 11. In yet other embodiments of Formula (II), c is 12. In some embodiments of Formula (II), c is 13. In other embodiments of Formula (II), c is 14. In more embodiments of Formula (II), c is 15. In yet other embodiments of Formula (II), c is 16.

In some certain embodiments of Formula (II), d is 0. In some embodiments of Formula (II), d is 1. In other embodiments of Formula (II), d is 2. In more embodiments of Formula (II), d is 3. In yet other embodiments of Formula (II), d is 4. In some embodiments of Formula (II), d is 5. In other embodiments of Formula (II), d is 6. In more embodiments of Formula (II), d is 7. In yet other embodiments of Formula (II), d is 8. In some embodiments of Formula (II), d is 9. In other embodiments of Formula (II), d is 10. In more embodiments of Formula (II), d is 11. In yet other embodiments of Formula (II), d is 12. In some embodiments of Formula (II), d is 13. In other embodiments of Formula (II), d is 14. In more embodiments of Formula (II), d is 15. In yet other embodiments of Formula (II), d is 16.

In some embodiments of Formula (II), e is 1. In other embodiments of Formula (II), e is 2. In more embodiments of Formula (II), e is 3. In yet other embodiments of Formula (II), e is 4. In some embodiments of Formula (II), e is 5. In other embodiments of Formula (II), e is 6. In more embodiments of Formula (II), e is 7. In yet other embodiments of Formula (II), e is 8. In some embodiments of Formula (II), e is 9. In other embodiments of Formula (II), e is 10. In more embodiments of Formula (II), e is 11. In yet other embodiments of Formula (II), e is 12.

In some embodiments of Formula (II), f is 1. In other embodiments of Formula (II), f is 2. In more embodiments of Formula (II), f is 3. In yet other embodiments of Formula (II), f is 4. In some embodiments of Formula (II), f is 5. In other embodiments of Formula (II), f is 6. In more embodiments of Formula (II), f is 7. In yet other embodiments of Formula (II), f is 8. In some embodiments of Formula (II), f is 9. In other embodiments of Formula (II), f is 10. In more embodiments of Formula (II), f is 11. In yet other embodiments of Formula (II), f is 12.

In some embodiments of Formula (II), g is 1. In other embodiments of Formula (II), g is 2. In more embodiments of Formula (II), g is 3. In yet other embodiments of Formula (II), g is 4. In some embodiments of Formula (II), g is 5. In other embodiments of Formula (II), g is 6. In more embodiments of Formula (II), g is 7. In yet other embodiments of Formula (II), g is 8. In some embodiments of Formula (II), g is 9. In other embodiments of Formula (II), g is 10. In more embodiments of Formula (II), g is 11. In yet other embodiments of Formula (II), g is 12.

In some embodiments of Formula (II), h is 1. In other embodiments of Formula (II), e is 2. In more embodiments of Formula (II), h is 3. In yet other embodiments of Formula (II), h is 4. In some embodiments of Formula (II), e is 5. In other embodiments of Formula (II), h is 6. In more embodiments of Formula (II), h is 7. In yet other embodiments of Formula (II), h is 8. In some embodiments of Formula (II), h is 9. In other embodiments of Formula (II), h is 10. In more embodiments of Formula (II), h is 11. In yet other embodiments of Formula (II), h is 12.

In some other various embodiments of Formula (II), a and d are the same. In some other embodiments, b and c are the same. In some other specific embodiments of Formula (II) a and d are the same and b and c are the same.

The sum of a and b and the sum of c and d are factors which may be varied to obtain a lipid having the desired properties. In one embodiment of Formula (II), a and b are chosen such that their sum is an integer ranging from 14 to 24. In other embodiments of Formula (II), c and d are chosen such that their sum is an integer ranging from 14 to 24. In a further embodiment of Formula (II), the sum of a and b and the sum of c and d are the same. For example, in some embodiments of Formula (II) the sum of a and b and the sum of c and d are both the same integer which may range from 14 to 24. In still more embodiments of Formula (II), a. b, c and d are selected such that the sum of a and b and the sum of c and d is 12 or greater.

The substituents at R^{1a}, R^{2a}, R^{3a} and R^{4a} are not particularly limited. In some embodiments, at least one of R^{1a}, R^{2a}, R^{3a} and R^{4a} is H. In certain embodiments of Formula (II) R^{1a}, R^{2a}, R^{3a} and R^{4a} are H at each occurrence. In certain other embodiments of Formula (II) at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments of Formula (II) at least one of R^{1a}, R^{2a}, R^{3a} and R^{4a} is C₁-C₆ alkyl. In some of the foregoing embodiments of Formula (II), the C₁-C₈ alkyl is methyl, ethyl, n-propyl, iso-propyl, n-butyl, iso-butyl, tert-butyl, n-hexyl or n-octyl.

In certain embodiments of the foregoing of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In certain embodiments of Formula (II), R^{1b} together with the carbon atom to which it is bound is taken together with an adjacent R^{1b} and the carbon atom to which it is bound to form a carbon-carbon double bond. In other embodiments of Formula (II) R^{4b} together with the carbon atom to which it is bound is taken together with an adjacent R^{4b} and the carbon atom to which it is bound to form a carbon-carbon double bond.

The substituents at R⁵ and R⁶ are not particularly limited in the foregoing embodiments. In certain embodiments of Formula (II) one of R⁵ or R⁶ is methyl. In other embodiments each of R⁵ or R⁶ is methyl.

The substituents at R⁷ are not particularly limited in the foregoing embodiments. In certain embodiments of Formula (II) R⁷ is C₆-C₁₆ alkyl. In some other embodiments, R⁷ is C₆-C₉ alkyl. In some of these embodiments of Formula (II), R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In various of the foregoing embodiments of Formula (II), R^{b} is branched C₁-C₁₅ alkyl. For example, in some embodiments of Formula (II) R^{b} has one of the following structures:

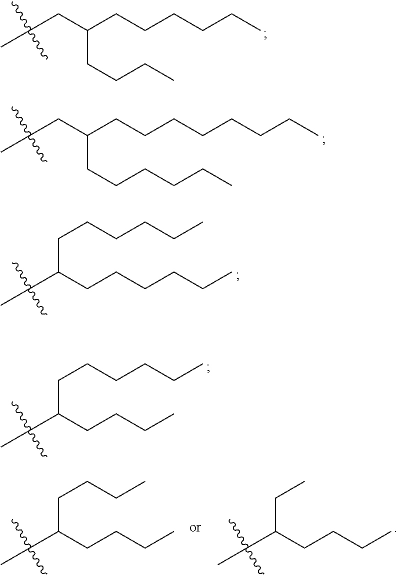

In certain other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of Formula (II), R⁸ and R⁹, together with the nitrogen atom to which they are attached, form a 5-membered heterocyclic ring, for example a pyrrolidinyl ring. In some different embodiments of Formula (II), R⁸ and R⁹, together with the nitrogen atom to which they are attached, form a 6-membered heterocyclic ring, for example a piperazinyl ring.

In still other embodiments of Formula (II), G³ is C₂-C₄ alkylene, for example C₃ alkylene.

In various different embodiments, the LNP comprises a compound having one of the structures set forth in Table II below.

TABLE II
Representative Lipid Compounds of Formula II

| | | Prep. |
|---|---|---|
| No. | Structure | Method* |
| II-1 | 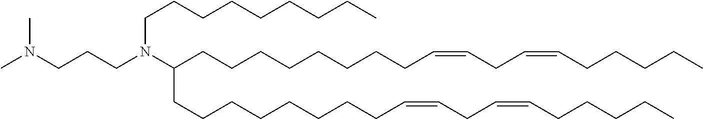 [SMILES: CCCCC/C=C\C/C=C\CCCCCCCCC(CCCCCCCC/C=C\C/C=C\CCCCC)N(CCCCCCCCC)CCCN(C)C] | D |
| II-2 | 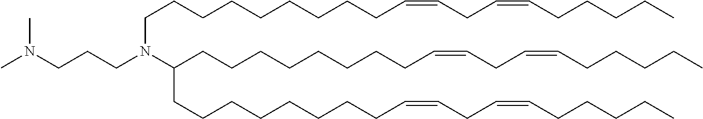 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | D |
| II-3 | 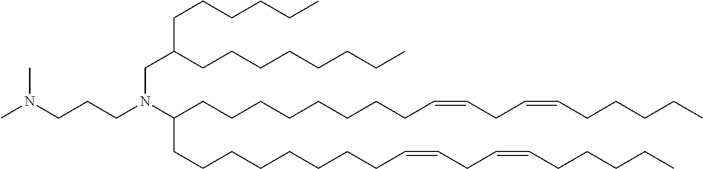 [SMILES: CCCCC/C=C\C/C=C\CCCCCCCCC(CCCCCCCC/C=C\C/C=C\CCCCC)N(CCCN(C)C)CC(CCCCCC)CCCCCCCC] | D |
| II-4 | 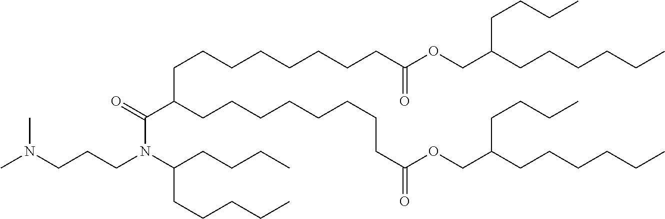 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | E |
| II-5 | 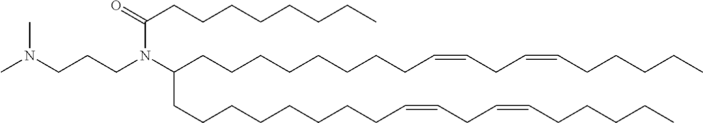 [SMILES: CCCCC/C=C\C/C=C\CCCCCCCCC(CCCCCCCC/C=C\C/C=C\CCCCC)N(CCCN(C)C)C(=O)CCCCCCCC] | D |
| II-6 | 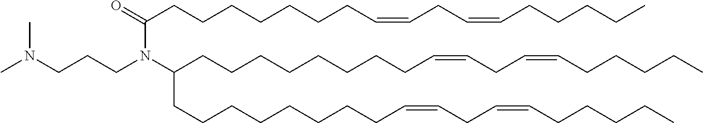 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | D |
| II-7 | 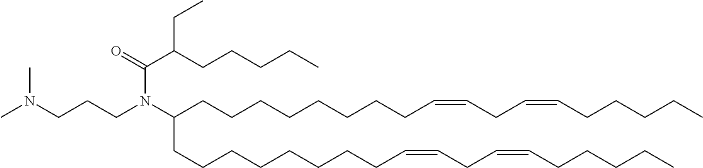 [SMILES: CCCCC/C=C\C/C=C\CCCCCCCCC(CCCCCCCC/C=C\C/C=C\CCCCC)N(CCCN(C)C)C(=O)C(CC)CCCCC] | D |
| II-8 | 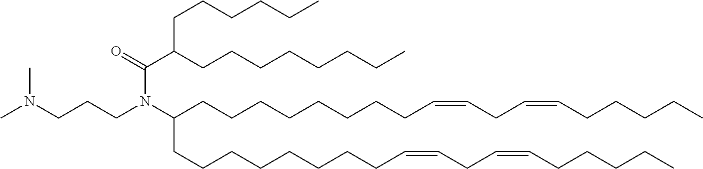 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | D |
| II-9 | 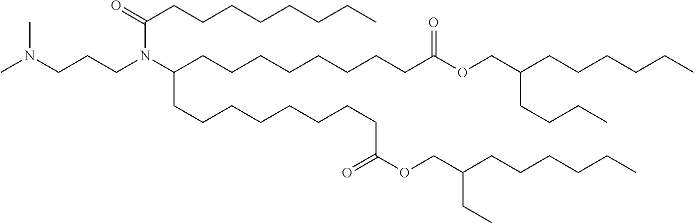 [SMILES: CCCCCCCCC(=O)N(CCCN(C)C)C(CCCCCCCCC(=O)OCC(CC)CCCCCC)CCCCCCCCC(=O)OCC(CCCC)CCCCCC] | D |
| II-10 | 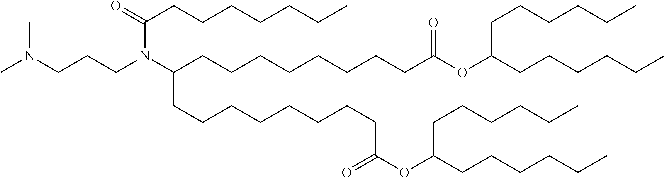 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-11 | 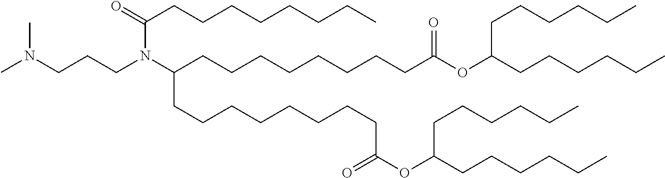 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-12 | 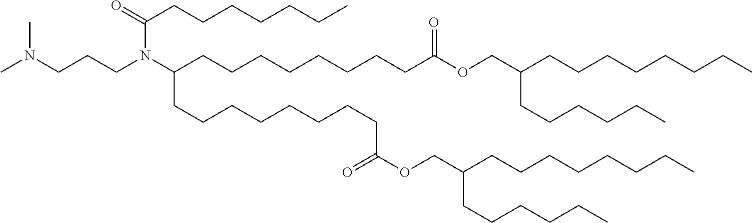 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | D |
| II-13 | 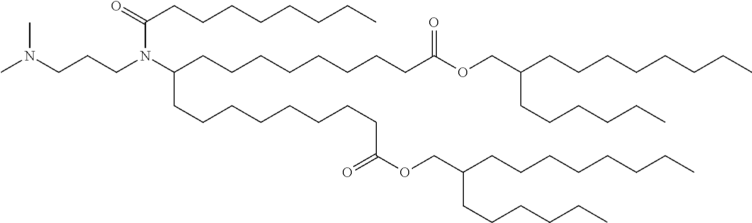 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | D |
| II-14 | 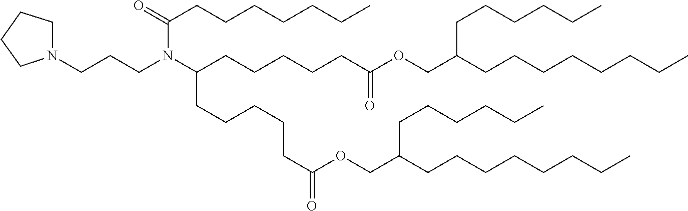 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | D |
| II-15 | 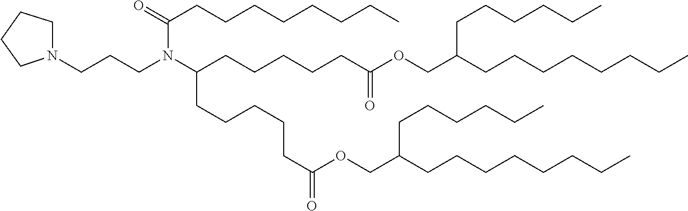 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC] | D |
| II-16 | 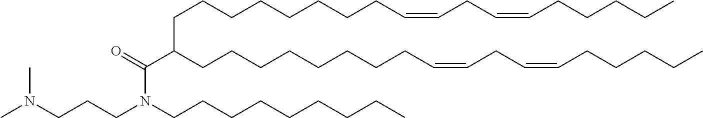 [SMILES: CCCCC/C=C\C/C=C\CCCCCCCCC(CCCCCCCC/C=C\C/C=C\CCCCC)C(=O)N(CCCCCCCCC)CCCN(C)C] | E |
| II-17 | 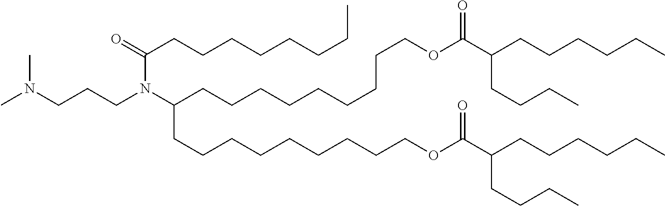 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] | D |
| II-18 | 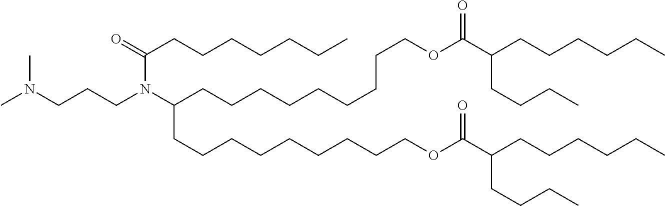 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | D |
| II-19 | 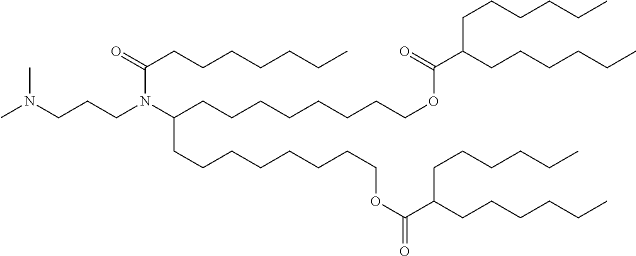 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-20 | 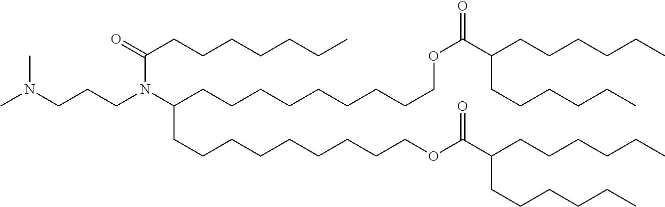 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-21 | 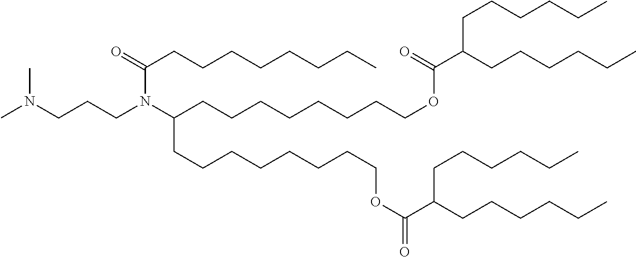 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-22 | 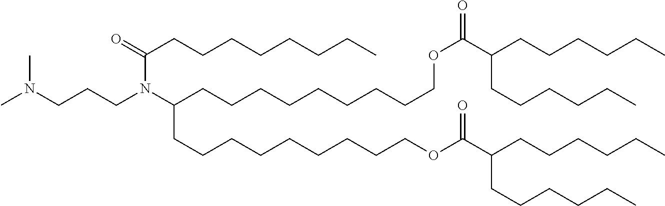 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-23 | 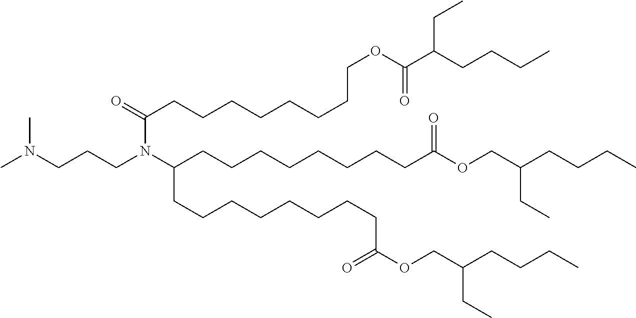 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | D |
| II-24 | 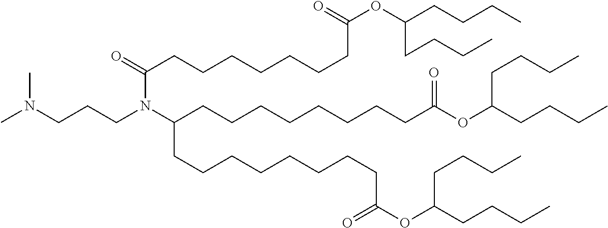 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | D |
| II-25 | 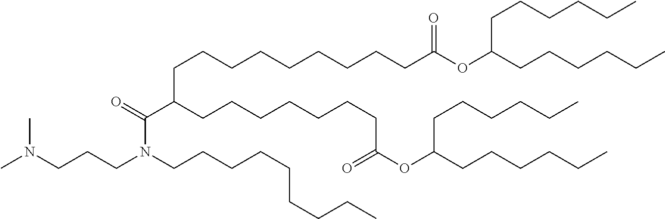 [SMILES: CCCCCCCCCN(CCCN(C)C)C(=O)C(CCCCCCCCCC(=O)OC(CCCCCC)CCCCCC)CCCCCCCC(=O)OC(CCCCCC)CCCCCC] | E |
| II-26 | 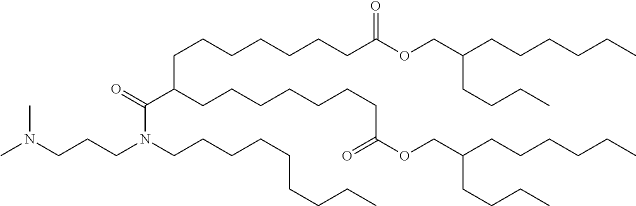 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | E |
| II-27 | 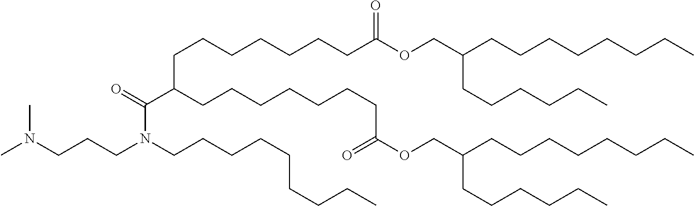 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | E |
| II-28 | 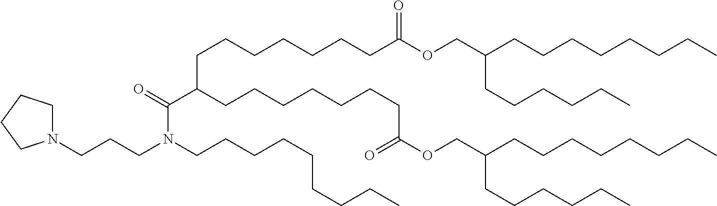 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | E |
| II-29 | 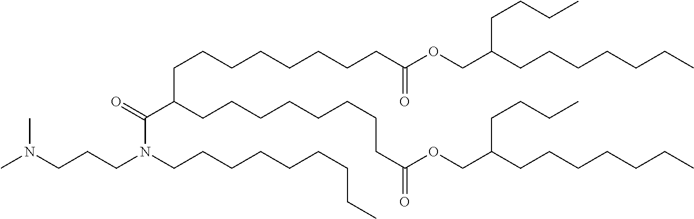 [SMILES: CCCCCCCCCN(CCCN(C)C)C(=O)C(CCCCCCCCC(=O)OCC(CCCC)CCCCCCC)CCCCCCCCC(=O)OCC(CCCC)CCCCCCC] | E |
| II-30 | 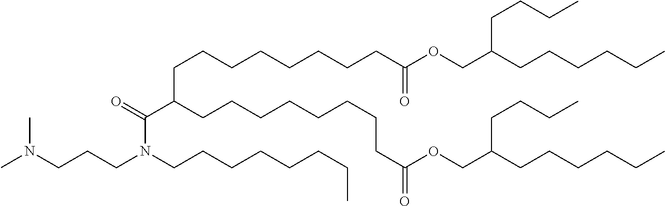 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-31 | 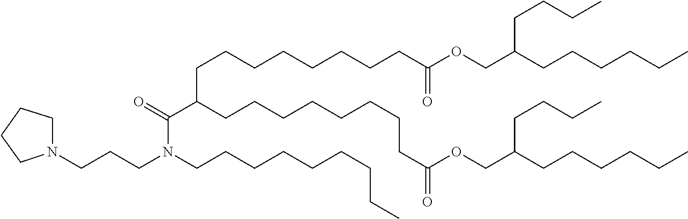 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-32 | 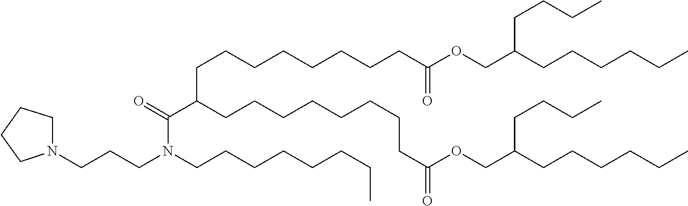 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-33 | 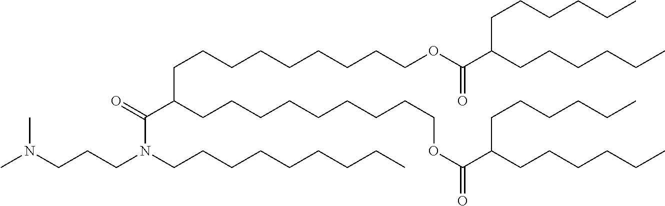 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | E |
| II-34 | 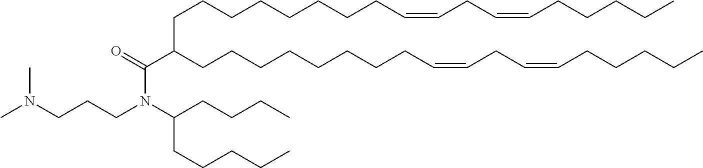 [SMILES: CCCCC/C=C\C/C=C\CCCCCCCCC(CCCCCCCC/C=C\C/C=C\CCCCC)C(=O)N(CCCN(C)C)C(CCCC)CCCCC] | E |
| II-35 | 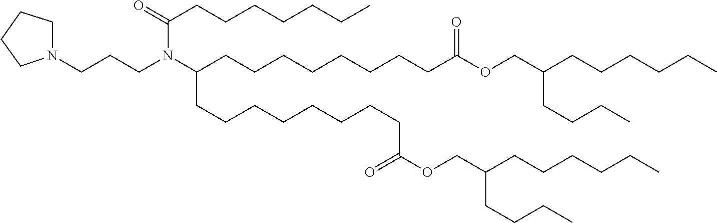 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] | D |
| II-36 | 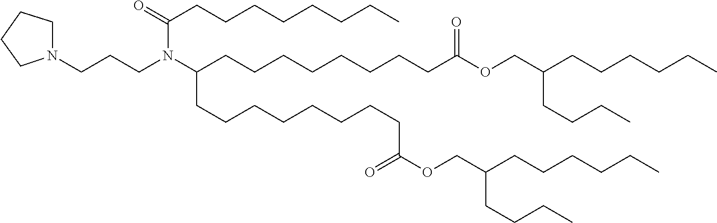 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] | D |

*Refers to general synthetic method for preparation of lipid

It is understood that any embodiment of the compounds of Formula (II), as set forth above, and any specific substituent and/or variable in the compound Formula (II), as set forth above, may be independently combined with other embodiments and/or substituents and/or variables of compounds of Formula (II) to form embodiments of the inventions not specifically set forth above. In addition, in the event that a list of substituents and/or variables is listed for any particular R group, L group, G group, or variables a-h, or x in a particular embodiment and/or claim, it is understood that each individual substituent and/or variable may be deleted from the particular embodiment and/or claim and that the remaining list of substituents and/or variables will be considered to be within the scope of the invention.

It is understood that in the present description, combinations of substituents and/or variables of the depicted formulae are permissible only if such contributions result in stable compounds.

In some embodiments, compositions comprising any one or more of the compounds of Formula (II) and a polynucleotide having activity as a gene editing/gene therapy reagent are provided. For example, in some embodiments, the compositions comprise any of the compounds of Formula (II) and a polynucleotide having activity as a gene editing/gene therapy reagent and one or more excipient selected from neutral lipids, steroids and polymer conjugated lipids. Other pharmaceutically acceptable excipients and/or carriers are also included in various embodiments of the compositions.

In some embodiments, the neutral lipid is selected from DSPC, DPPC, DMPC, DOPC, POPC, DOPE and SM. In some embodiments, the neutral lipid is DSPC. In various embodiments, the molar ratio of the compound to the neutral lipid ranges from about 2:1 to about 8:1.

In various embodiments, the compositions further comprise a steroid or steroid analogue. In certain embodiments, the steroid or steroid analogue is cholesterol. In some of these embodiments, the molar ratio of the compound to cholesterol ranges from about 2:1 to 1:1.

In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the compound to the pegylated lipid ranges from about 100:1 to about 25:1.

In some embodiments, the LNPs comprise a polynucleotide having activity as a gene therapy reagent and a lipid compound having the following Formula (III):

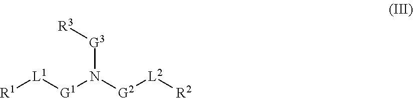

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;

G¹ and G² are each independently unsubstituted C₁-C₁₂ alkylene or C₁-C₁₂ alkenylene;

G³ is C₁-C₂₄ alkylene, C₁-C₂₄ alkenylene, C₃-C₈ cycloalkylene, C₃-C₈ cycloalkenylene;

R^{a} is H or C₁-C₁₂ alkyl;

R¹ and R² are each independently C₆-C₂₄ alkyl or C₆-C₂₄ alkenyl;

R³ is H, OR⁵, CN, —C(═O)OR⁴, —OC(═O)R⁴ or —NR⁵C(═O)R⁴;

E R⁴ is C₁-C₁₂ alkyl;

R⁵ is H or C₁-C₆ alkyl; and

x is 0, 1 or 2.

In some of the foregoing embodiments of Formula (III), the compound has one of the following structures (IIIA) or (IIIB)

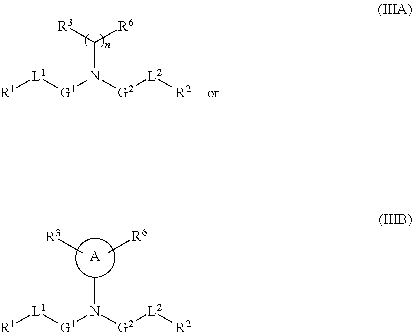

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the compound has structure (IIIA), and in other embodiments of Formula (III), the compound has structure (IIIB).

In other embodiments of Formula (III), the compound has one of the following structures (IIIC) or (IIID):

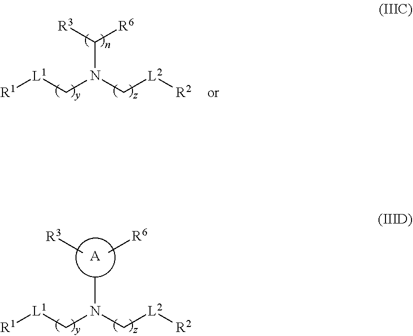

wherein y and z are each independently integers ranging from 1 to 12.

In any of the foregoing embodiments of Formula (III), one of L¹ or L² is —O(C═O)—. For example, in some embodiments of Formula (III) each of L¹ and L² are —O(C═O)—. In some different embodiments of Formula (III), L¹ and L² are each independently —(C═O)O— or —O(C═O)—. For example, in some embodiments of Formula (III) each of L¹ and L² is —(C═O)O—.

In some different embodiments of Formula (III), the compound has one of the following structures (IIIE) or (IIIF):

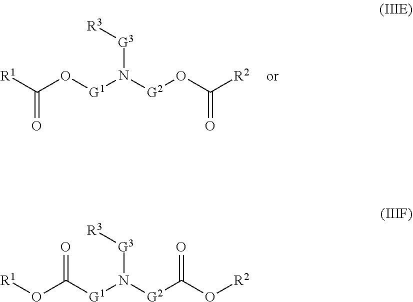

In some of the foregoing embodiments of Formula (III), the compound has one of the following structures (IIIG), (IIIH), (III I), or (IIIJ):

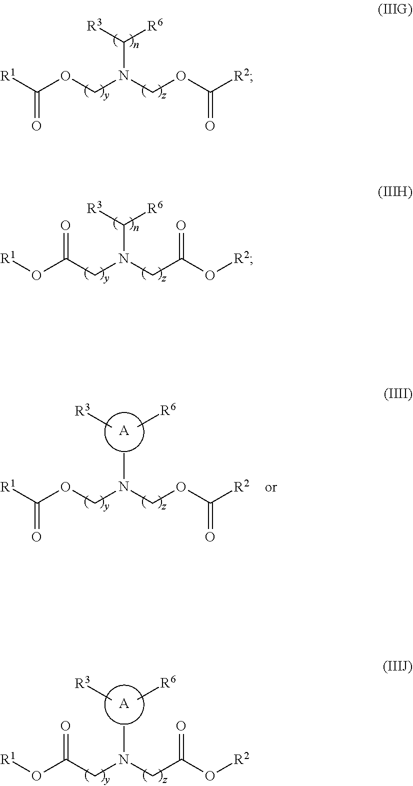

In some of the foregoing embodiments of Formula (III), n is an integer ranging from 2 to 12, for example from 2 to 8 or from 2 to 4. For example, in some embodiments of Formula (III), n is 3, 4, 5 or 6. In some embodiments of Formula (III), n is 3. In some embodiments of Formula (III), n is 4. In some embodiments of Formula (III), n is 5. In some embodiments of Formula (III), n is 6.

In some other of the foregoing embodiments of Formula (III), y and z are each independently an integer ranging from 2 to 10. For example, in some embodiments of Formula (III), y and z are each independently an integer ranging from 4 to 9 or from 4 to 6.

In some of the foregoing embodiments of Formula (III), R⁶ is H. In other of the foregoing embodiments of Formula (III), R⁶ is C₁-C₂₄ alkyl. In other embodiments of Formula (III), R⁶ is OH.

In some embodiments of Formula (III), G³ is unsubstituted. In other embodiments of Formula (III), G3 is substituted. In various different embodiments of Formula (III), G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments of Formula (III), R¹ and R² each, independently have the following structure:

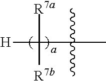

wherein: R^{7a} and R^{7b} are, at each occurrence, independently H or C₁-C₁₂ alkyl; and a is an integer from 2 to 12, wherein R^{7a}, R^{7b} and a are each selected such that R¹ and R² each independently comprise from 6 to 20 carbon atoms. For example, in some embodiments of Formula (III) a is an integer ranging from 5 to 9 or from 8 to 12.

In some of the foregoing embodiments of Formula (III), at least one occurrence of R^{7a} is H. For example, in some embodiments of Formula (III), R^{7a} is H at each occurrence. In other different embodiments of Formula (III), at least one occurrence of R^{7b} is C₁-C₈ alkyl. For example, in some embodiments of Formula (III), C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

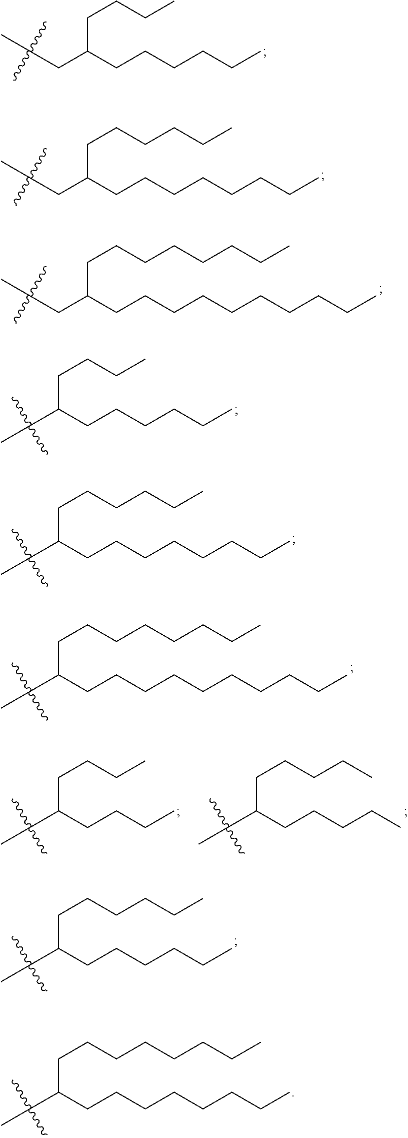

In some of the foregoing embodiments of Formula (III), R³ is OH, CN, —C(═O)OR⁴, —OC(═O)R⁴ or —NHC(═O)R⁴. In some embodiments of Formula (III), R⁴ is methyl or ethyl.

In various different embodiments of Formula (III), the LNP comprises a compound having one of the structures set forth in Table III below.

TABLE III
Representative Lipid Compounds of Formula (III)

| | | Prep. |
|---|---|---|
| No. | Structure | Method* |
| III-1 | 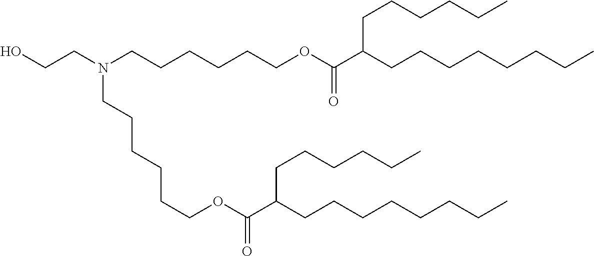 [SMILES: CCCCCCCCC(CCCCCC)C(=O)OCCCCCCN(CCO)CCCCCCOC(=O)C(CCCCCC)CCCCCCCC] | F |
| III-2 | 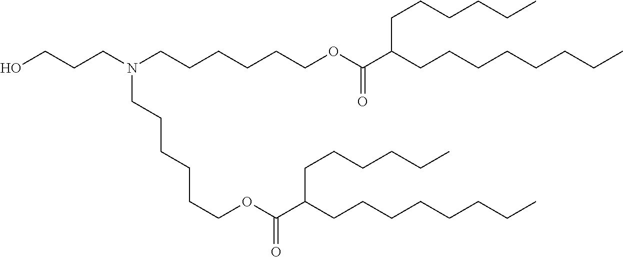 [SMILES: CCCCCCCCC(CCCCCC)C(=O)OCCCCCCN(CCCO)CCCCCCOC(=O)C(CCCCCC)CCCCCCCC] | F |
| III-3 | 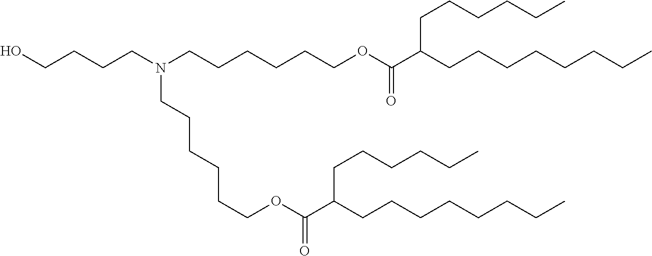 [SMILES: CCCCCCCCC(CCCCCC)C(=O)OCCCCCCN(CCCCO)CCCCCCOC(=O)C(CCCCCC)CCCCCCCC] | F |
| III-4 | 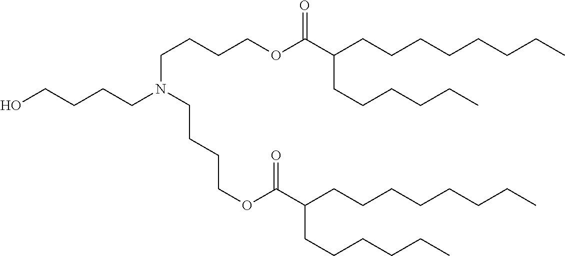 [SMILES: CCCCCCCCC(CCCCCC)C(=O)OCCCCN(CCCCO)CCCCOC(=O)C(CCCCCC)CCCCCCCC] | F |
| III-5 | 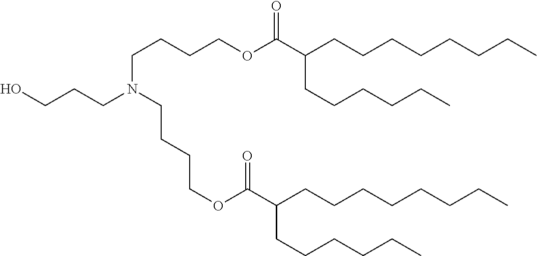 [SMILES: CCCCCCCCC(CCCCCC)C(=O)OCCCCN(CCCO)CCCCOC(=O)C(CCCCCC)CCCCCCCC] | F |
| III-6 | 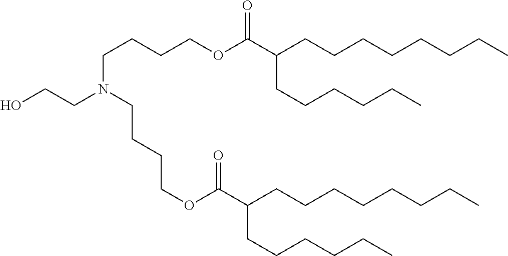 [SMILES: CCCCCCCCC(CCCCCC)C(=O)OCCCCN(CCO)CCCCOC(=O)C(CCCCCC)CCCCCCCC] | F |
| III-7 | 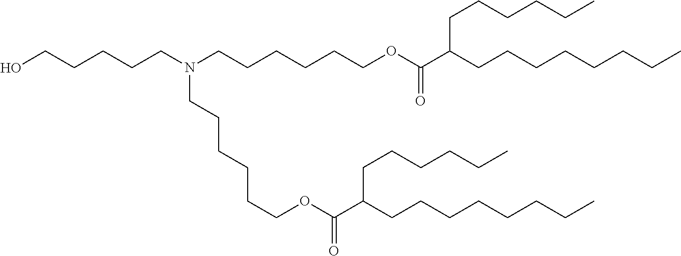 [SMILES: CCCCCCCCC(CCCCCC)C(=O)OCCCCCCN(CCCCCO)CCCCCCOC(=O)C(CCCCCC)CCCCCCCC] | F |
| III-8 | 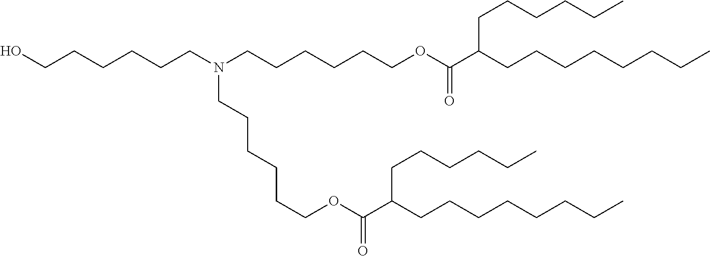 [SMILES: CCCCCCCCC(CCCCCC)C(=O)OCCCCCCN(CCCCCCO)CCCCCCOC(=O)C(CCCCCC)CCCCCCCC] | F |
| III-9 | 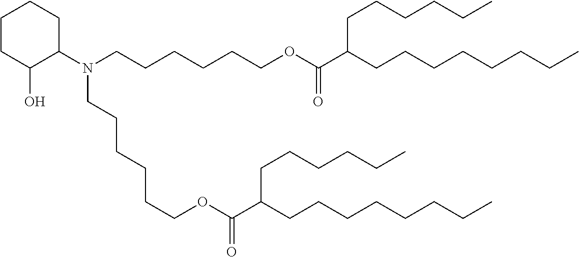 [SMILES: CCCCCCCCC(CCCCCC)C(=O)OCCCCCCN(CCCCCCOC(=O)C(CCCCCC)CCCCCCCC)C1CCCCC1O] | F |
| III-10 | 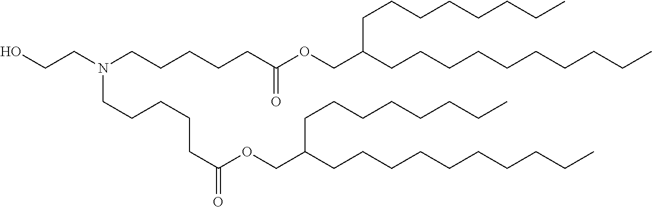 [SMILES: CCCCCCCCCCC(CCCCCCCC)COC(=O)CCCCCN(CCO)CCCCCC(=O)OCC(CCCCCCCC)CCCCCCCCCC] | F |
| III-11 | 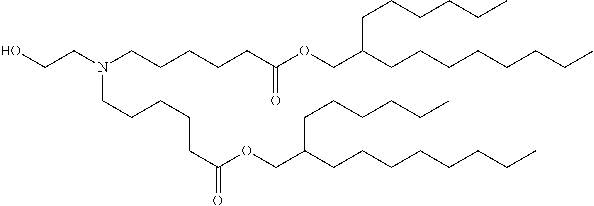 [SMILES: CCCCCCCCC(CCCCCC)COC(=O)CCCCCN(CCO)CCCCCC(=O)OCC(CCCCCC)CCCCCCCC] | F |
| III-12 | 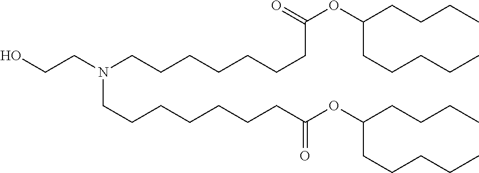 [SMILES: CCCCCC(CCCC)OC(=O)CCCCCCCN(CCO)CCCCCCCC(=O)OC(CCCC)CCCCC] | F |
| III-13 | 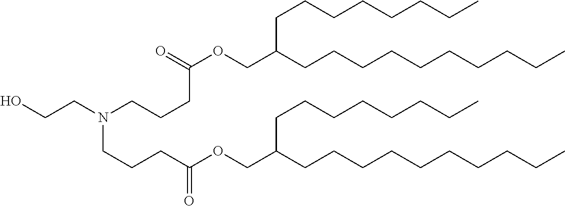 [SMILES: CCCCCCCCCCC(CCCCCCCC)COC(=O)CCCN(CCO)CCCC(=O)OCC(CCCCCCCC)CCCCCCCCCC] | F |
| III-14 | 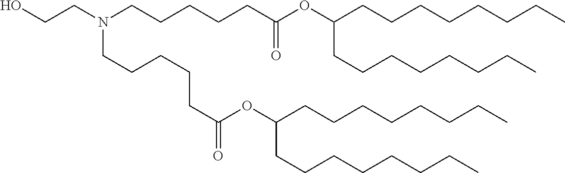 [SMILES: CCCCCCCCC(CCCCCCCC)OC(=O)CCCCCN(CCO)CCCCCC(=O)OC(CCCCCCCC)CCCCCCCC] | F |
| III-15 | 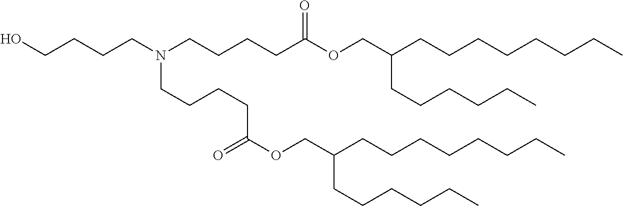 [SMILES: CCCCCCCCC(CCCCCC)COC(=O)CCCCN(CCCCO)CCCCC(=O)OCC(CCCCCC)CCCCCCCC] | F |
| III-16 | 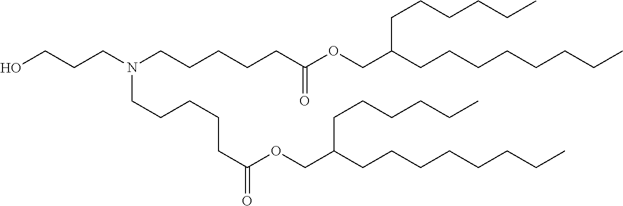 [SMILES: CCCCCCCCC(CCCCCC)COC(=O)CCCCCN(CCCO)CCCCCC(=O)OCC(CCCCCC)CCCCCCCC] | F |
| III-17 | 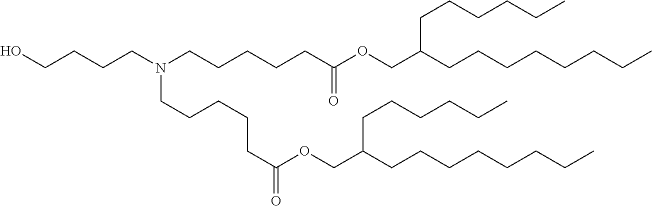 [SMILES: CCCCCCCCC(CCCCCC)COC(=O)CCCCCN(CCCCO)CCCCCC(=O)OCC(CCCCCC)CCCCCCCC] | F |
| III-18 | 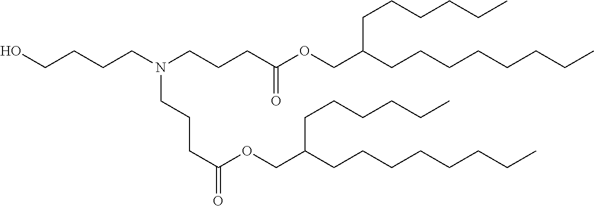 [SMILES: CCCCCCCCC(CCCCCC)COC(=O)CCCN(CCCCO)CCCC(=O)OCC(CCCCCC)CCCCCCCC] | F |
| III-19 | 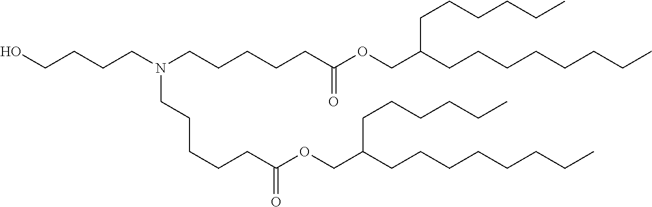 [SMILES: CCCCCCCCC(CCCCCC)COC(=O)CCCCCN(CCCCO)CCCCCC(=O)OCC(CCCCCC)CCCCCCCC] | F |
| III-20 | 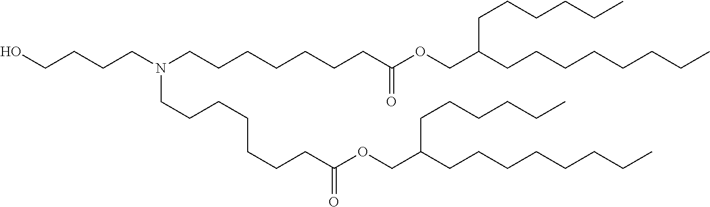 [SMILES: CCCCCCCCC(CCCCCC)COC(=O)CCCCCCCN(CCCCO)CCCCCCCC(=O)OCC(CCCCCC)CCCCCCCC] | F |
| III-21 | 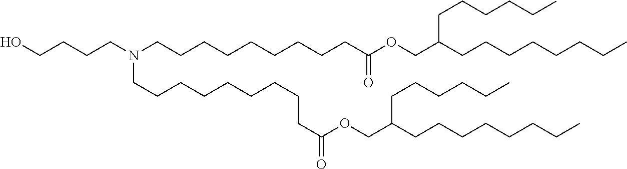 [SMILES: CCCCCCCCC(CCCCCC)COC(=O)CCCCCCCCCN(CCCCO)CCCCCCCCCC(=O)OCC(CCCCCC)CCCCCCCC] | F |
| III-22 | 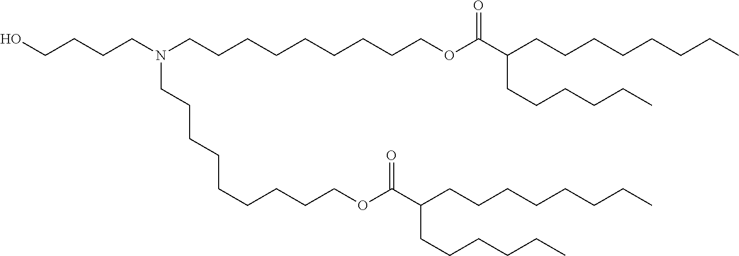 [SMILES: CCCCCCCCC(CCCCCC)C(=O)OCCCCCCCCCN(CCCCO)CCCCCCCCCOC(=O)C(CCCCCC)CCCCCCCC] | F |
| III-23 | 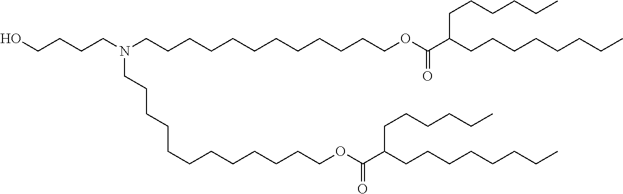 [SMILES: CCCCCCCCC(CCCCCC)C(=O)OCCCCCCCCCCCCN(CCCCO)CCCCCCCCCCCCOC(=O)C(CCCCCC)CCCCCCCC] | F |
| III-24 | 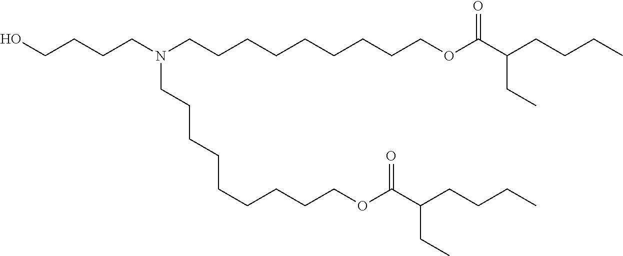 [SMILES: CCCCC(CC)C(=O)OCCCCCCCCCN(CCCCO)CCCCCCCCCOC(=O)C(CC)CCCC] | F |
| III-25 | 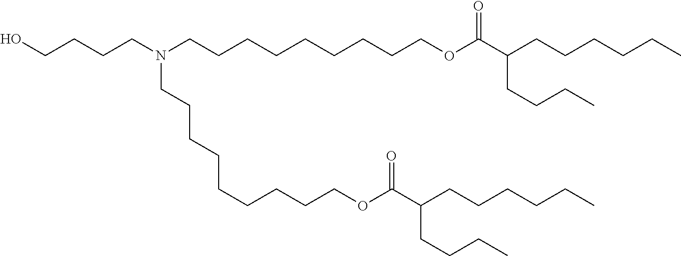 [SMILES: CCCCCCC(CCCC)C(=O)OCCCCCCCCCN(CCCCO)CCCCCCCCCOC(=O)C(CCCC)CCCCCC] | F |
| III-26 | 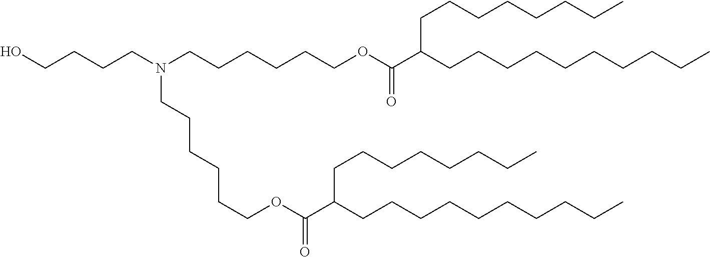 [SMILES: CCCCCCCCCCC(CCCCCCCC)C(=O)OCCCCCCN(CCCCO)CCCCCCOC(=O)C(CCCCCCCC)CCCCCCCCCC] | F |
| III-27 | 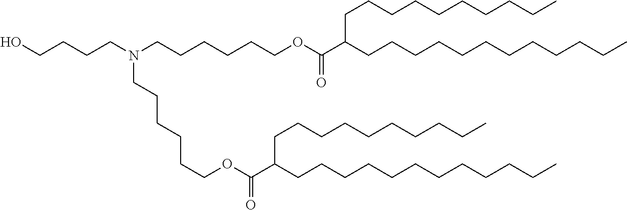 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | F |
| III-28 | 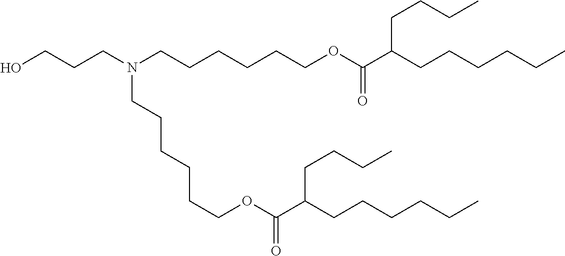 [SMILES: CCCCCCC(CCCC)C(=O)OCCCCCCN(CCCO)CCCCCCOC(=O)C(CCCC)CCCCCC] | F |
| III-29 | 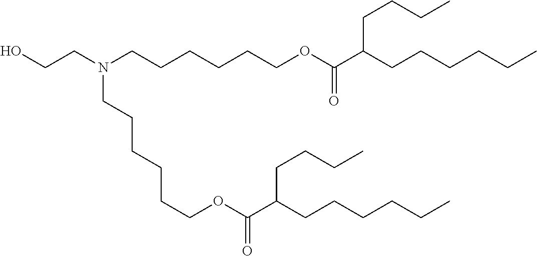 [SMILES: CCCCCCC(CCCC)C(=O)OCCCCCCN(CCO)CCCCCCOC(=O)C(CCCC)CCCCCC] | F |
| III-30 | 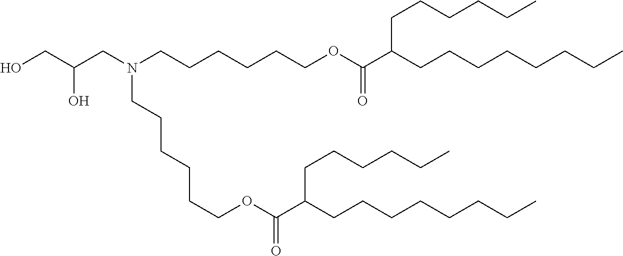 [SMILES: CCCCCCCCC(CCCCCC)C(=O)OCCCCCCN(CCCCCCOC(=O)C(CCCCCC)CCCCCCCC)CC(O)CO] | F |
| III-31 | 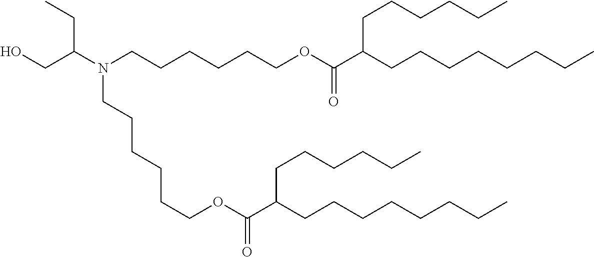 [SMILES: CCCCCCCCC(CCCCCC)C(=O)OCCCCCCN(CCCCCCOC(=O)C(CCCCCC)CCCCCCCC)C(CC)CO] | F |
| III-32 | 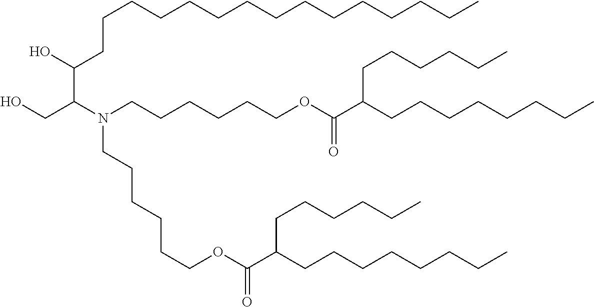 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | F |
| III-33 | 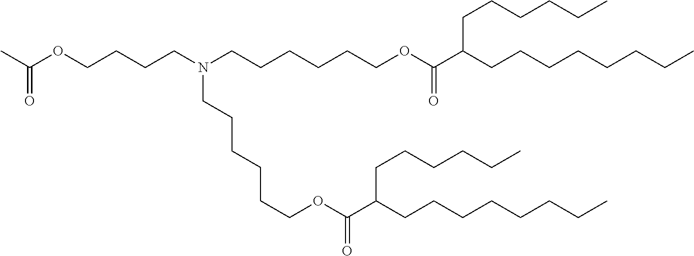 [SMILES: CCCCCCCCC(CCCCCC)C(=O)OCCCCCCN(CCCCCCOC(=O)C(CCCCCC)CCCCCCCC)CCCCOC(C)=O] | F |
| III-34 | 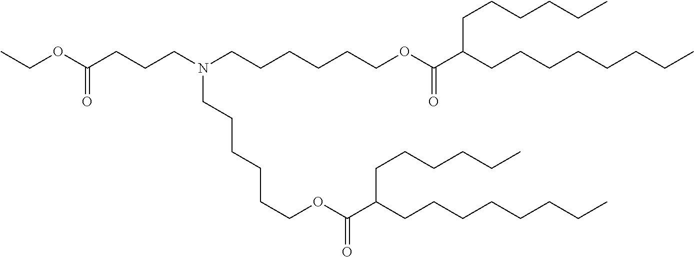 [SMILES: CCCCCCCCC(CCCCCC)C(=O)OCCCCCCN(CCCCCCOC(=O)C(CCCCCC)CCCCCCCC)CCCC(=O)OCC] | F |
| III-35 | 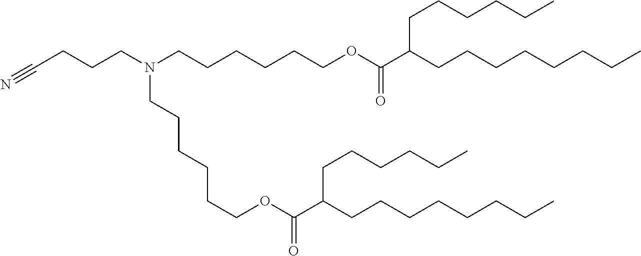 [SMILES: CCCCCCCCC(CCCCCC)C(=O)OCCCCCCN(CCCC#N)CCCCCCOC(=O)C(CCCCCC)CCCCCCCC] | F |
| III-36 | 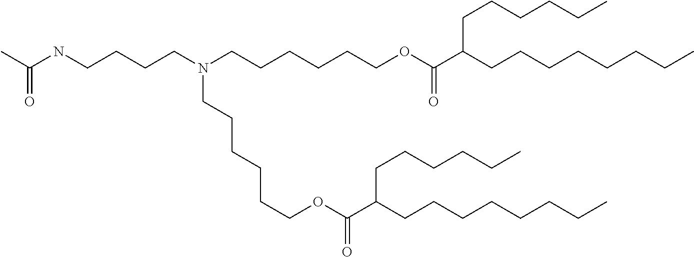 [SMILES: CCCCCCCCC(CCCCCC)C(=O)OCCCCCCN(CCCCCCOC(=O)C(CCCCCC)CCCCCCCC)CCCCNC(C)=O] | F |
| III-37 | 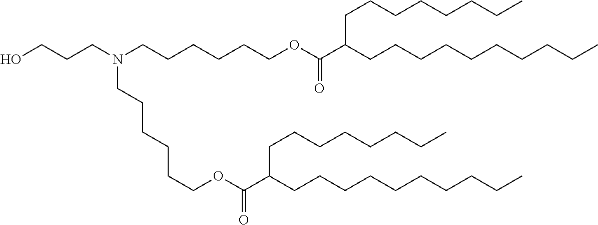 [SMILES: CCCCCCCCCCC(CCCCCCCC)C(=O)OCCCCCCN(CCCO)CCCCCCOC(=O)C(CCCCCCCC)CCCCCCCCCC] | F |
| III-38 | 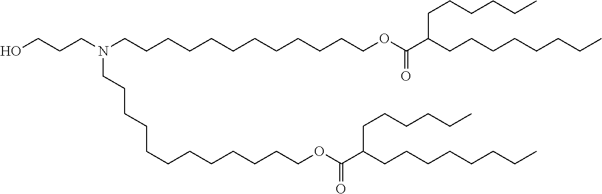 [SMILES: CCCCCCCCC(CCCCCC)C(=O)OCCCCCCCCCCCCN(CCCO)CCCCCCCCCCCCOC(=O)C(CCCCCC)CCCCCCCC] | F |
| III-39 | 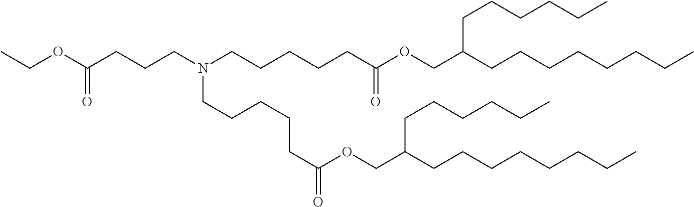 [SMILES: CCCCCCCCC(CCCCCC)COC(=O)CCCCCN(CCCCCC(=O)OCC(CCCCCC)CCCCCCCC)CCCC(=O)OCC] | F |
| III-40 | 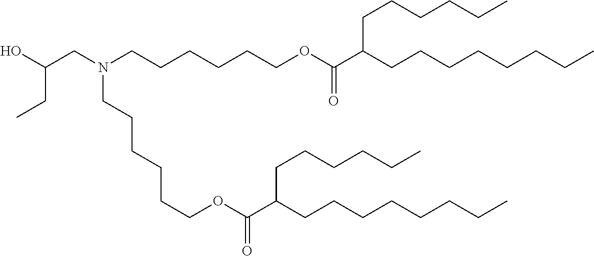 [SMILES: CCCCCCCCC(CCCCCC)C(=O)OCCCCCCN(CCCCCCOC(=O)C(CCCCCC)CCCCCCCC)CC(O)CC] | F |
| III-41 | 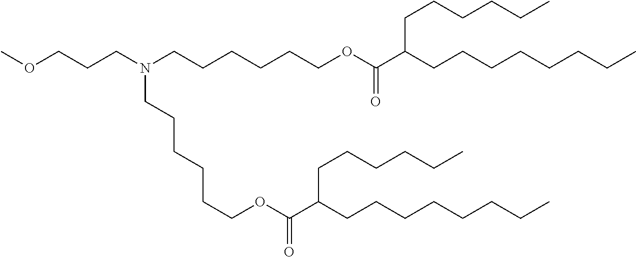 [SMILES: CCCCCCCCC(CCCCCC)C(=O)OCCCCCCN(CCCCCCOC(=O)C(CCCCCC)CCCCCCCC)CCCOC] | F |
| III-42 | 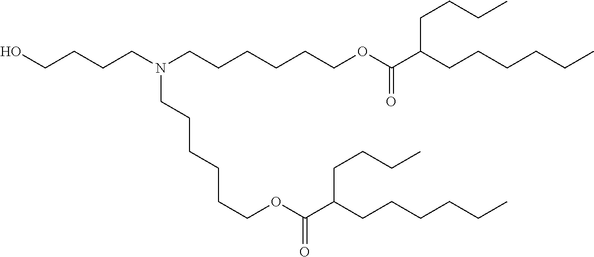 [SMILES: CCCCCCC(CCCC)C(=O)OCCCCCCN(CCCCO)CCCCCCOC(=O)C(CCCC)CCCCCC] | F |
| III-43 | 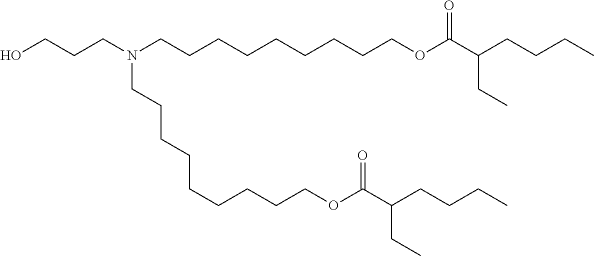 [SMILES: CCCCC(CC)C(=O)OCCCCCCCCCN(CCCO)CCCCCCCCCOC(=O)C(CC)CCCC] | F |
| III-44 | 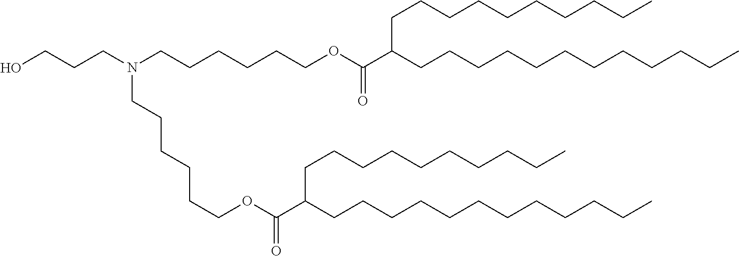 [SMILES: CCCCCCCCCCCCC(CCCCCCCCCC)C(=O)OCCCCCCN(CCCO)CCCCCCOC(=O)C(CCCCCCCCCC)CCCCCCCCCCCC] | F |
| III-45 | 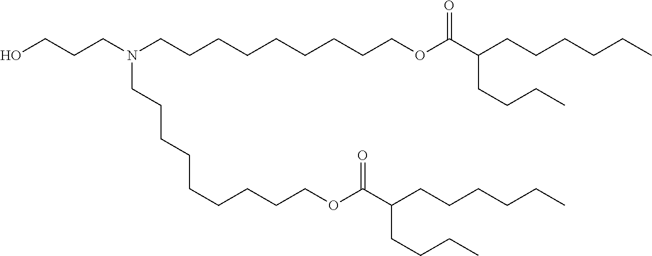 [SMILES: CCCCCCC(CCCC)C(=O)OCCCCCCCCCN(CCCO)CCCCCCCCCOC(=O)C(CCCC)CCCCCC] | F |
| III-46 | 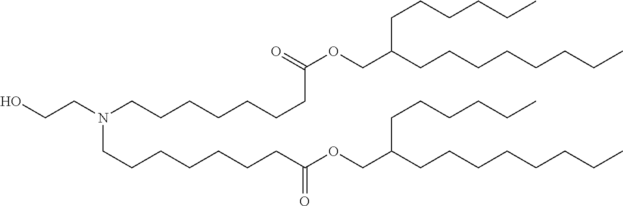 [SMILES: CCCCCCCCC(CCCCCC)COC(=O)CCCCCCCN(CCO)CCCCCCCC(=O)OCC(CCCCCC)CCCCCCCC] | F |
| III-47 | 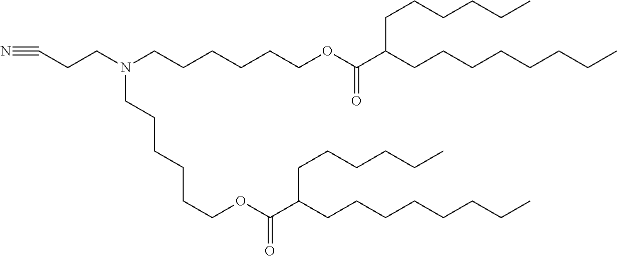 [SMILES: CCCCCCCCC(CCCCCC)C(=O)OCCCCCCN(CCC#N)CCCCCCOC(=O)C(CCCCCC)CCCCCCCC] | F |
| III-48 | 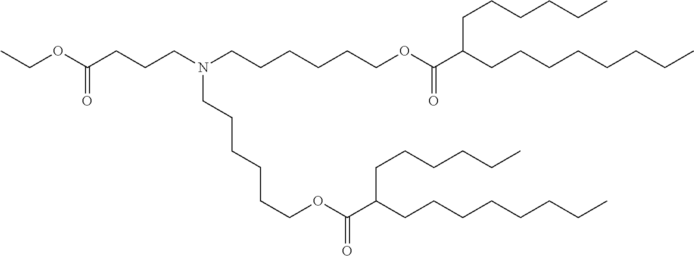 [SMILES: CCCCCCCCC(CCCCCC)C(=O)OCCCCCCN(CCCCCCOC(=O)C(CCCCCC)CCCCCCCC)CCCC(=O)OCC] | F |
| III-49 | 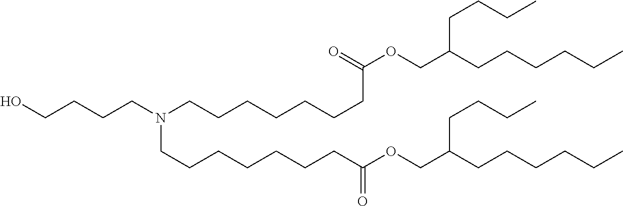 [SMILES: CCCCCCC(CCCC)COC(=O)CCCCCCCN(CCCCO)CCCCCCCC(=O)OCC(CCCC)CCCCCC] | F |

*Refers to general synthetic method for preparation of lipid

It is understood that any embodiment of the compounds of structure (III), as set forth above, and any specific substituent and/or variable in the compound structure (III), as set forth above, may be independently combined with other embodiments and/or substituents and/or variables of compounds of structure (III) to form embodiments of the inventions not specifically set forth above. In addition, in the event that a list of substituents and/or variables is listed for any particular R group, L group, G group, A group, or variables a, n, x, y, or z in a particular embodiment and/or claim, it is understood that each individual substituent and/or variable may be deleted from the particular embodiment and/or claim and that the remaining list of substituents and/or variables will be considered to be within the scope of the invention.

It is understood that in the present description, combinations of substituents and/or variables of the depicted formulae are permissible only if such contributions result in stable compounds.

In some embodiments, compositions comprising any one or more of the compounds of structure (III) and a polynucleotide having activity as a gene editing/gene therapy reagent are provided. For example, in some embodiments, the LNPs comprise any of the compounds of structure (III) and a polynucleotide having activity as a gene editing/gene therapy reagent and one or more excipient selected from neutral lipids, steroids and polymer conjugated lipids. Other pharmaceutically acceptable excipients and/or carriers are also included in various embodiments of the compositions.

In some embodiments, the neutral lipid is selected from DSPC, DPPC, DMPC, DOPC, POPC, DOPE and SM. In some embodiments, the neutral lipid is DSPC. In various embodiments, the molar ratio of the compound to the neutral lipid ranges from about 2:1 to about 8:1.

In various embodiments, the compositions further comprise a steroid or steroid analogue. In certain embodiments, the steroid or steroid analogue is cholesterol. In some of these embodiments, the molar ratio of the compound to cholesterol ranges from about 5:1 to 1:1.

In various embodiments of the LNPs disclosed herein, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the compound to the pegylated lipid ranges from about 100:1 to about 20:1.

In some embodiments, the LNPs comprise a polynucleotide having activity as a gene therapy reagent and a lipid compound having the following Formula (IV):

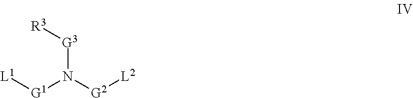

or a pharmaceutically acceptable salt, tautomer, prodrug or stereoisomer thereof, wherein:

L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{c}C(═O)NR^{e}R^{f}, —OC(═O)NR^{e}R^{f};

—NR^{d}C(═O)OR² or a direct bond;

G¹ and G² are each independently C₂-C₁₂ alkylene or C₂-C₁₂ alkenylene;

G³ is C₁-C₂₄ alkylene, C₂-C₂₄ alkenylene, C₃-C₈ cycloalkylene or C₃-C₈ cycloalkenylene;

R^{a}, R^{b}, R^{d} and R^{e} are each independently H or C₁-C₁₂ alkyl or C₂-C₁₂ alkenyl;

R^{c} and R^{f} are each independently C₁-C₁₂ alkyl or C₂-C₁₂ alkenyl;

R¹ and R² are each independently branched C₆-C₂₄ alkyl or branched C₆-C₂₄ alkenyl;

R³ is —C(═O)N(R⁴)R⁵ or —C(═O)OR⁶;

R⁴ is C₁-C₁₂ alkyl;

R⁵ is H or C₁-C₈ alkyl or C₂-C₈ alkenyl;

R⁶ is H, aryl or aralkyl; and

x is 0, 1 or 2. In certain embodiments of Formula (IV), G³ is unsubstituted. In more specific embodiments G³ is C₂-C₁₂ alkylene, for example, in some embodiments G³ is C₃-C₇ alkylene or in other embodiments G³ is C₃-C₁₂ alkylene.

In some of the foregoing embodiments of Formula (IV), the compound has the following structure (IVA):

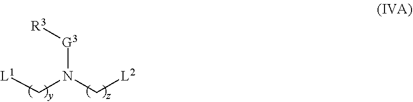

wherein y and z are each independently integers ranging from 2 to 12.

In some of the foregoing embodiments of Formula (IV), L¹ is —O(C═O)R¹, —(C═O)OR¹ or —C(═O)NR^{b}R^{c}, and L² is —O(C═O)R², —(C═O)OR² or —C(═O)NR^{e}R^{f}. For example, in some embodiments each of L¹ and L² is —(C═O)O—. In other embodiments L¹ is —(C═O)OR¹ and L² is —C(═O)NR^{e}R^{f}.

In other embodiments of the foregoing compounds of Formula (IV), the compound has one of the following structures (IVB), (IVC) or (IVD):

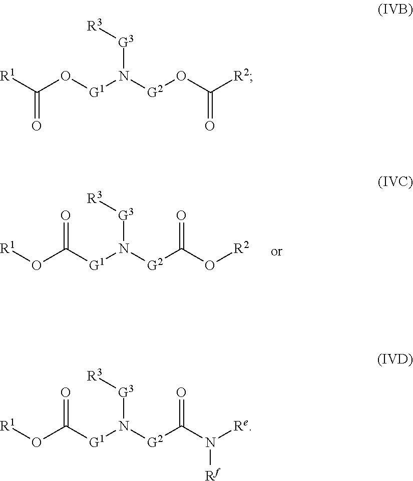

In some of the foregoing embodiments, the compound has structure (IVB), in other embodiments, the compound has structure (IVC) and in still other embodiments the compound has the structure (VID).

In some different embodiments of the foregoing, the compound has one of the following structures (IVE), (VIF) or (IVG):

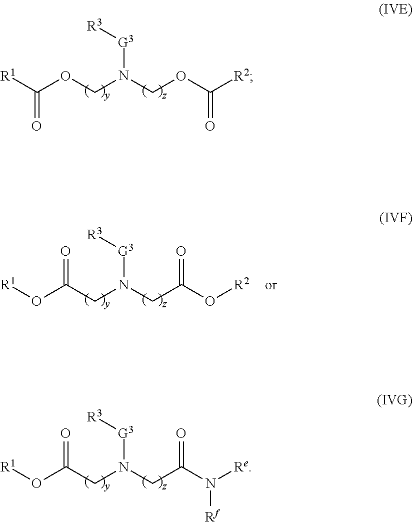

wherein y and z are each independently integers ranging from 2 to 12.

In some of the foregoing embodiments of Formula (IV), y and z are each independently an integer ranging from 2 to 10, 2 to 8, from 4 to 10 or from 4 to 7. For example, in some embodiments of Formula (IV), y is 4, 5, 6, 7, 8, 9, 10, 11 or 12. In some embodiments of Formula (IV), z is 4, 5, 6, 7, 8, 9, 10, 11 or 12. In some embodiments of Formula (IV), y and z are the same, while in other embodiments y and z are different.

In some of the foregoing embodiments of Formula (IV), R¹ or R², or both is branched C₆-C₂₄ alkyl. For example, in some embodiments of Formula (IV), R¹ and R² each, independently have the following structure:

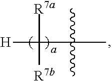

wherein:

R^{7a} and R^{7b} are, at each occurrence, independently H or C₁-C₁₂ alkyl; and a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (IV), at least one occurrence of R^{7a} is H. For example, in some embodiments of Formula (IV), R^{7a} is H at each occurrence. In other different embodiments of the foregoing compounds of Formula (IV), at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (IV), R¹ or R², or both, has one of the following structures

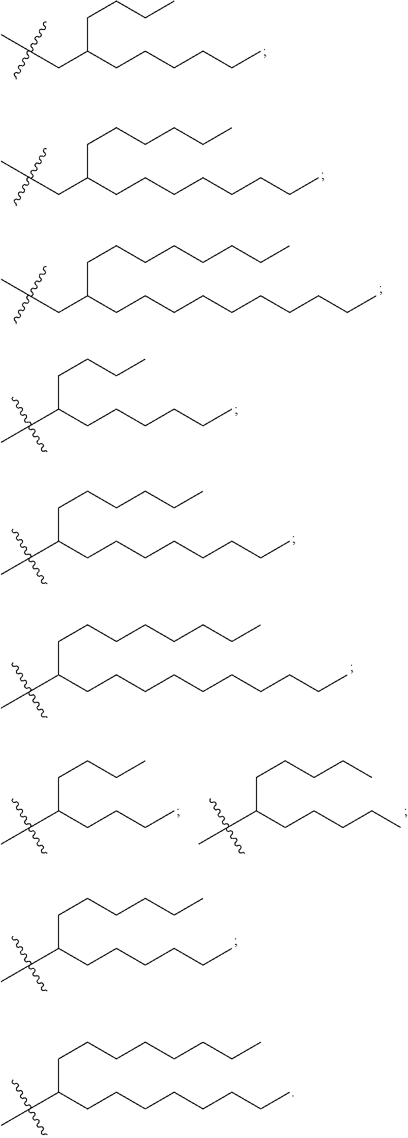

In some of the foregoing embodiments of Formula (IV), R^{b}, R^{c}, R^{e} and R^{f} are each independently C₃-C₁₂ alkyl. For example, in some embodiments of Formula (IV) R^{b}, R^{c}, R^{e} and R^{f} are n-hexyl and in other embodiments R^{b}, R^{c}, R^{e} and R^{f} are n-octyl.

In some of the foregoing embodiments of Formula (IV), R³ is —C(═O)N(R⁴)R⁵. In more specific embodiments of Formula (IV), R⁴ is ethyl, propyl, n-butyl, n-hexyl, n-octyl or n-nonyl. In certain embodiments of Formula (IV), R⁵ is H, methyl, ethyl, propyl, n-butyl, n-hexyl or n-octyl.

In some embodiments of Formula (IV), R³ is —C(═O)OR⁶. In certain embodiments of Formula (IV), R⁶ is benzyl and in other embodiments R⁶ is H.

In some of the foregoing embodiments of Formula (IV), R⁴, R⁵ and R⁶ are independently optionally substituted with one or more substituents selected from the group consisting of —OR^{g}, —NR^{g}C(═O)R^{h}, —C(═O)NR^{g}R^{h}, —C(═O)R^{h}, —OC(═O)R^{h}, —C(═O)OR^{h} and —OR^{h}OH, wherein:

R^{g} is, at each occurrence independently H or C₁-C₆ alkyl;

R^{h} is at each occurrence independently C₁-C₆ alkyl; and

Rⁱ is, at each occurrence independently C₁-C₆ alkylene.

In certain specific embodiments of Formula (IV), R³ has one of the following structures:

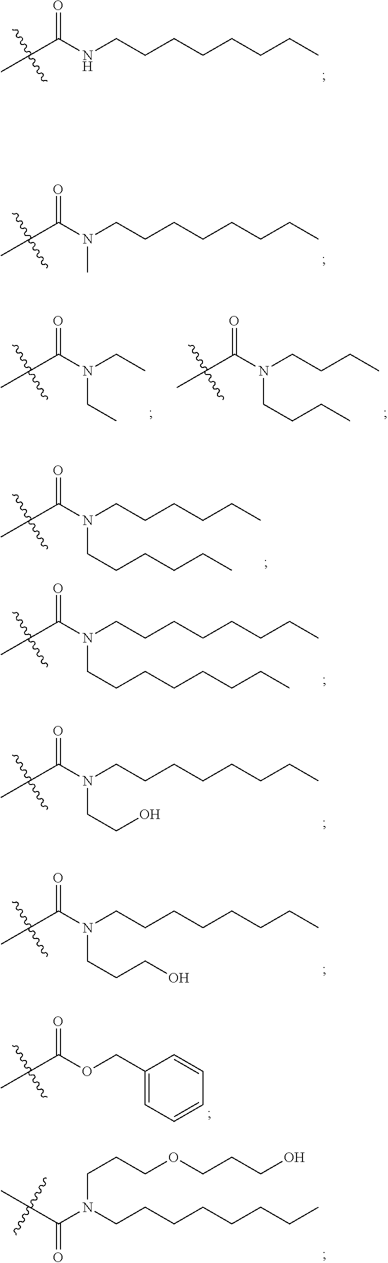

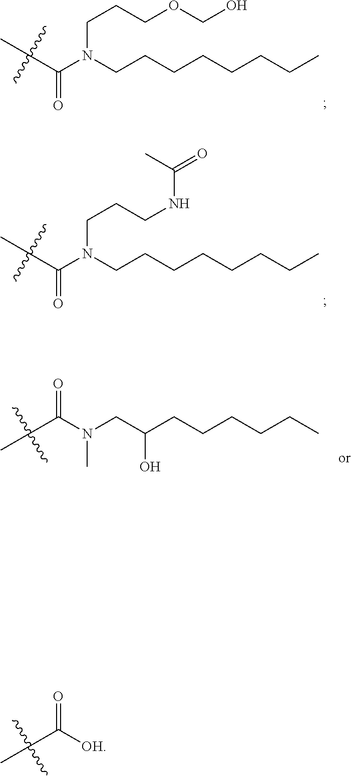

In various different embodiments of Formula (IV), the compound has one of the structures set forth in Table IV below.

TABLE IV
Representative Lipid Compounds of Formula (IV)

| | | Preparation |
|---|---|---|
| No. | Structure | Method |
| 1 | 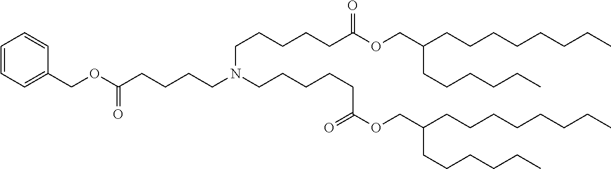 [SMILES: CCCCCCCCC(CCCCCC)COC(=O)CCCCCN(CCCCCC(=O)OCC(CCCCCC)CCCCCCCC)CCCCC(=O)OCc1ccccc1] | G |
| 2 | 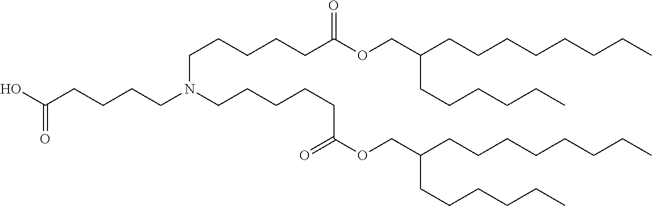 [SMILES: CCCCCCCCC(CCCCCC)COC(=O)CCCCCN(CCCCCC(=O)OCC(CCCCCC)CCCCCCCC)CCCCC(=O)O] | G |
| 3 | 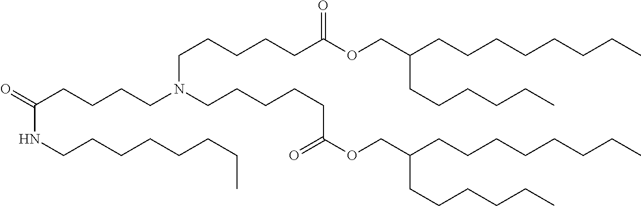 [SMILES: CCCCCCCCNC(=O)CCCCN(CCCCCC(=O)OCC(CCCCCC)CCCCCCCC)CCCCCC(=O)OCC(CCCCCC)CCCCCCCC] | G |
| 4 | 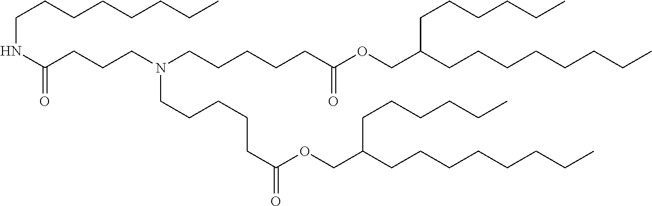 [SMILES: CCCCCCCCNC(=O)CCCN(CCCCCC(=O)OCC(CCCCCC)CCCCCCCC)CCCCCC(=O)OCC(CCCCCC)CCCCCCCC] | G |
| 5 | 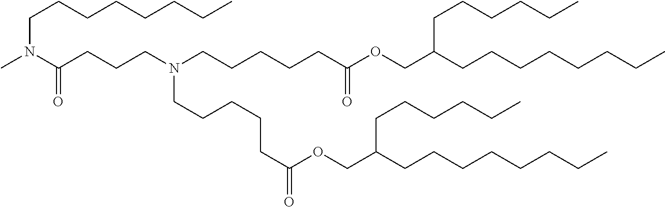 [SMILES: CCCCCCCCC(CCCCCC)COC(=O)CCCCCN(CCCCCC(=O)OCC(CCCCCC)CCCCCCCC)CCCC(=O)N(C)CCCCCCCC] | G |
| 6 | 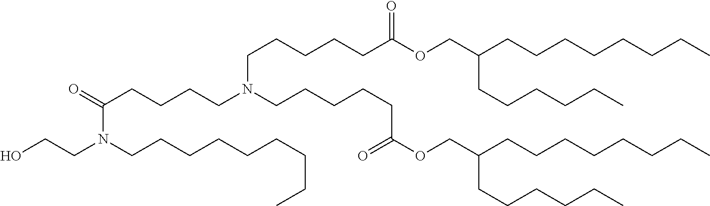 [SMILES: CCCCCCCCCN(CCO)C(=O)CCCCN(CCCCCC(=O)OCC(CCCCCC)CCCCCCCC)CCCCCC(=O)OCC(CCCCCC)CCCCCCCC] | G |
| 7 | 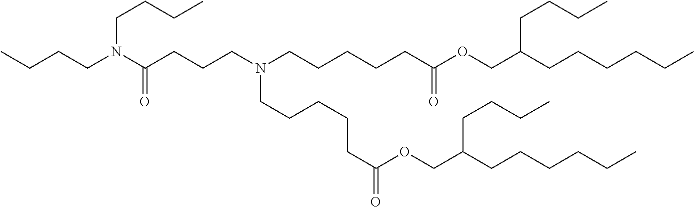 [SMILES: CCCCCCC(CCCC)COC(=O)CCCCCN(CCCCCC(=O)OCC(CCCC)CCCCCC)CCCC(=O)N(CCCC)CCCC] | H |
| 8 | 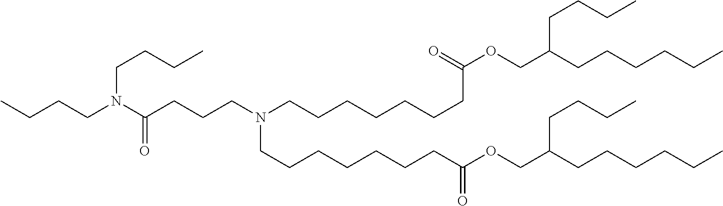 [SMILES: CCCCCCC(CCCC)COC(=O)CCCCCCCN(CCCCCCCC(=O)OCC(CCCC)CCCCCC)CCCC(=O)N(CCCC)CCCC] | H |
| 9 | 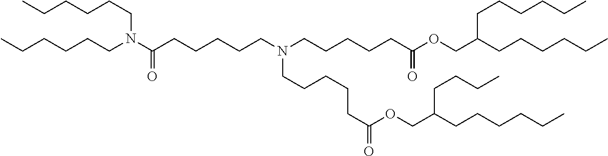 [SMILES: CCCCCCC(CCCC)COC(=O)CCCCCN(CCCCCC(=O)OCC(CCCCCC)CCCCCC)CCCCCC(=O)N(CCCCCC)CCCCCC] | I |
| 10 | 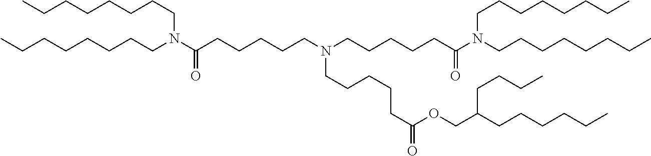 [SMILES: CCCCCCCCN(CCCCCCCC)C(=O)CCCCCN(CCCCCC(=O)OCC(CCCC)CCCCCC)CCCCCC(=O)N(CCCCCCCC)CCCCCCCC] | I |
| 11 | 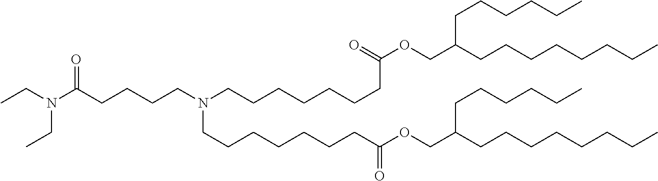 [SMILES: CCCCCCCCC(CCCCCC)COC(=O)CCCCCCCN(CCCCCCCC(=O)OCC(CCCCCC)CCCCCCCC)CCCCC(=O)N(CC)CC] | G |
| 12 | 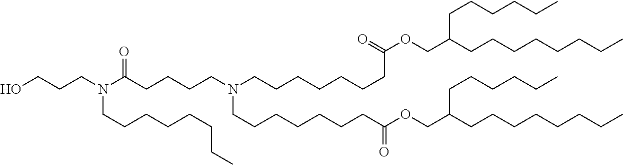 [SMILES: CCCCCCCCC(CCCCCC)COC(=O)CCCCCCCN(CCCCCCCC(=O)OCC(CCCCCC)CCCCCCCC)CCCCC(=O)N(CCCO)CCCCCCCC] | G |
| 13 | 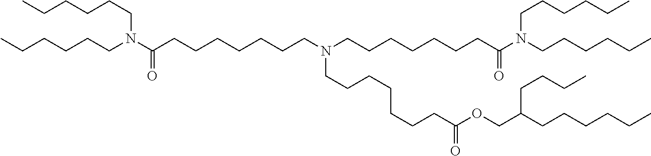 [SMILES: CCCCCCC(CCCC)COC(=O)CCCCCCCN(CCCCCCCC(=O)N(CCCCCC)CCCCCC)CCCCCCCC(=O)N(CCCCCC)CCCCCC] | I |
| 14 | 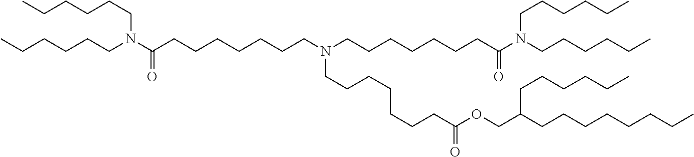 [SMILES: CCCCCCCCC(CCCCCC)COC(=O)CCCCCCCN(CCCCCCCC(=O)N(CCCCCC)CCCCCC)CCCCCCCC(=O)N(CCCCCC)CCCCCC] | I |
| 15 | 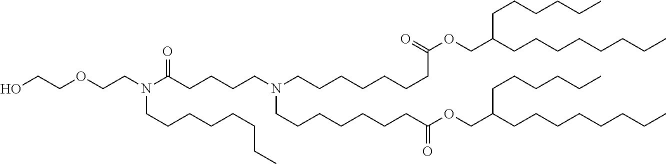 [SMILES: CCCCCCCCC(CCCCCC)COC(=O)CCCCCCCN(CCCCCCCC(=O)OCC(CCCCCC)CCCCCCCC)CCCCC(=O)N(CCCCCCCC)CCOCCO] | G |
| 16 | 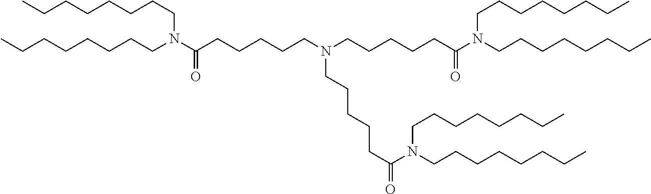 [SMILES: CCCCCCCCN(CCCCCCCC)C(=O)CCCCCN(CCCCCC(=O)N(CCCCCCCC)CCCCCCCC)CCCCCC(=O)N(CCCCCCCC)CCCCCCCC] | I |
| 17 | 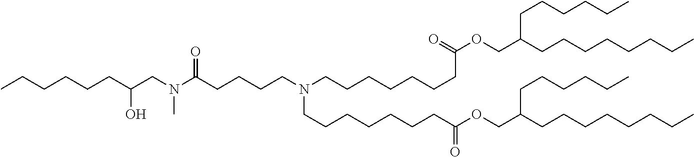 [SMILES: CCCCCCCCC(CCCCCC)COC(=O)CCCCCCCN(CCCCCCCC(=O)OCC(CCCCCC)CCCCCCCC)CCCCC(=O)N(C)CC(O)CCCCCC] | G |

It is understood that any embodiment of the compounds of Formula (IV), as set forth above, and any specific substituent and/or variable in the compound of Formula (IV), as set forth above, may be independently combined with other embodiments and/or substituents and/or variables of compounds of Formula (IV) to form embodiments of the inventions not specifically set forth above. In addition, in the event that a list of substituents and/or variables is listed for any particular R group, L group, G group, or variables a, x, y, or z in a particular embodiment and/or claim, it is understood that each individual substituent and/or variable may be deleted from the particular embodiment and/or claim and that the remaining list of substituents and/or variables will be considered to be within the scope of the invention.

It is understood that in the present description, combinations of substituents and/or variables of the depicted formulae are permissible only if such contributions result in stable compounds.

In some embodiments, compositions comprising any one or more of the compounds of Formula (IV) and a polynucleotide having activity as a gene editing/gene therapy reagent are provided. For example, in some embodiments, the compositions comprise any of the compounds of Formula (IV) and a polynucleotide having activity as a gene therapy reagent and one or more excipient selected from neutral lipids, steroids and polymer conjugated lipids. Other pharmaceutically acceptable excipients and/or carriers are also included in various embodiments of the compositions.

In some embodiments, the neutral lipid is selected from DSPC, DPPC, DMPC, DOPC, POPC, DOPE and SM. In some embodiments, the neutral lipid is DSPC. In various embodiments, the molar ratio of the compound to the neutral lipid ranges from about 2:1 to about 8:1.

In various embodiments, the compositions further comprise a steroid or steroid analogue. In certain embodiments, the steroid or steroid analogue is cholesterol. In some of these embodiments, the molar ratio of the compound to cholesterol ranges from about 5:1 to 1:1.

In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the compound to the pegylated lipid ranges from about 100:1 to about 20:1.

In some embodiments, the LNP comprises a pegylated lipid having the following Formula (V):

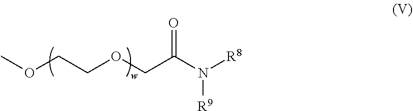

or a pharmaceutically acceptable salt, tautomer or stereoisomer thereof, wherein:
R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

In some embodiments of Formula (V), R⁸ and R⁹ are each independently straight, saturated alkyl chains containing from 12 to 16 carbon atoms. In other embodiments, the average w is about 45.

In other embodiments of Formula (V), the average w ranges from 42 to 55. For example, 42, 43, 44, 45, 46, 47, 48, 49, 50, 51, 52, 53, 54 or 55. In some specific embodiments, w is about 49.

In some embodiments, the pegylated lipid has the following structure (Va):

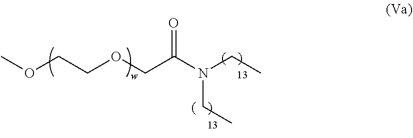

wherein the average w is about 49.

### Delivery

For the purposes of administration, the LNPs of the present invention may be administered as a raw chemical or may be formulated as pharmaceutical compositions. Pharmaceutical compositions of the present invention comprise an LNP comprising a therapeutic agent, such as a polynucleotide having activity as a gene therapy reagent, a compound of structure (I), (II), (III) and/or (IV) and one or more pharmaceutically acceptable carrier, diluent or excipient. The compound of structure (I), (II), (III) and/or (IV) is present in the composition in an amount which is effective to form a lipid nanoparticle and deliver the therapeutic agent, e.g., for treating a particular disease or condition of interest. Appropriate concentrations and dosages can be readily determined by one skilled in the art.

The proteins (e.g., nucleases), polynucleotides and/or compositions comprising the proteins and/or polynucleotides described herein may be delivered to a target cell by any suitable means, including, for example, by injection of an LNP comprising the protein and/or mRNA components.

Suitable cells include but are not limited to eukaryotic and prokaryotic cells and/or cell lines. Non-limiting examples of such cells or cell lines generated from such cells include T-cells, COS, CHO (e.g., CHO-S, CHO-K1, CHO-DG44, CHO-DUXB 11, CHO-DUKX, CHOK1SV), VERO, MDCK, WI38, V79, B14AF28-G3, BHK, HaK, NS0, SP2/0-Agl4, HeLa, HEK293 (e.g., HEK293-F, HEK293-H, HEK293-T), and perC6 cells as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Suitable cells also include stem cells such as, by way of example, embryonic stem cells, induced pluripotent stem cells (iPS cells), hematopoietic stem cells, neuronal stem cells and mesenchymal stem cells.

Methods of delivering proteins comprising DNA-binding domains also include those described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

DNA binding domains and fusion proteins comprising these DNA binding domains as described herein may also be delivered using vectors containing sequences encoding one or more of the DNA-binding protein(s). Additionally, additional nucleic acids (e.g., donors) also may be delivered via these vectors. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences and/or additional nucleic acids as appropriate. Thus, when one or more DNA-binding proteins as described herein are introduced into the cell, and additional DNAs as appropriate, they may be carried on the same vector or on different vectors. When multiple vectors are used, each vector may comprise a sequence encoding one or multiple DNA-binding proteins and additional nucleic acids as desired.

Conventional viral and non-viral based gene transfer methods can be used to introduce nucleic acids encoding engineered DNA-binding proteins in cells (e.g., mammalian cells) and target tissues and to co-introduce additional nucleotide sequences as desired. Such methods can also be used to administer nucleic acids (e.g., encoding DNA-binding proteins and/or donors) to cells in vitro. In certain embodiments, nucleic acids are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, lipid nanoparticles, immunoliposomes, polycation or lipid:nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. No. 5,049,386, U.S. Pat. No. 4,946,787; and U.S. Pat. No. 4,897,355) and lipofection reagents are sold commercially (e.g., Transfectam™, Lipofectin™, and Lipofectamine™ RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al (2009) Nature Biotechnology 27(7) p. 643).

The use of RNA or DNA viral based systems for the delivery of nucleic acids encoding engineered DNA-binding proteins, and/or donors as desired takes advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS USA 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS USA 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1): 10-20 (1997); Dranoff et al., Hum. Gene Ther. 1:111-2 (1997).

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6, AAV8, AAV8.2, AAV9 and AAVrh10 and pseudotyped AAV such as AAV2/8, AAV2/5 and AAV2/6 can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In addition, AAV can be manufactured using a baculovirus system (see e.g. U.S. Pat. Nos. 6,723,551 and 7,271,002).

Purification of AAV particles from a 293 or baculovirus system typically involves growth of the cells which produce the virus, followed by collection of the viral particles from the cell supernatant or lysing the cells and collecting the virus from the crude lysate. AAV is then purified by methods known in the art including ion exchange chromatography (e.g. see U.S. Pat. Nos. 7,419,817 and 6,989,264), ion exchange chromatography and CsCl density centrifugation (e.g. PCT publication WO2011094198A10), immunoaffinity chromatography (e.g. WO2016128408) or purification using AVB Sepharose (e.g. GE Healthcare Life Sciences).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., (Proc. Natl. Acad. Sci. USA 92:9747-9751 (1995)), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Delivery methods for CRISPR/Cas systems can comprise those methods described above. For example, in animal models, in vitro transcribed Cas encoding mRNA or recombinant Cas protein can be directly injected into one-cell stage embryos using glass needles to genome-edited animals. To express Cas and guide RNAs in cells in vitro, typically plasmids that encode them are transfected into cells via lipofection or electroporation. Also, recombinant Cas protein can be complexed with in vitro transcribed guide RNA where the Cas-guide RNA ribonucleoprotein is taken up by the cells of interest (Kim et al (2014) Genome Res 24(6):1012). For therapeutic purposes, Cas and guide RNAs can be delivered by a combination of viral and non-viral techniques. For example, mRNA encoding Cas may be delivered via nanoparticle delivery while the guide RNAs and any desired transgene or repair template are delivered via AAV (Yin et al (2016) Nat Biotechnol 34(3) p. 328).

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

Ex vivo cell transfection for diagnostics, research, transplant or for gene therapy (e.g., via re-infusion of the transfected cells into the host organism) is well known to those of skill in the art. In a preferred embodiment, cells are isolated from the subject organism, transfected with a DNA-binding proteins nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., J. Exp. Med. 176:1693-1702 (1992)).

Stem cells that have been modified may also be used in some embodiments. For example, neuronal stem cells that have been made resistant to apoptosis may be used as therapeutic compositions where the stem cells also contain the ZFP TFs of the invention. Resistance to apoptosis may come about, for example, by knocking out BAX and/or BAK using BAX- or BAK-specific ZFNs (see, U.S. patent application Ser. No. 12/456,043) in the stem cells, or those that are disrupted in a caspase, again using caspase-6 specific ZFNs for example.

Vectors (e.g., retroviruses, adenoviruses, liposomes, etc.) containing therapeutic DNA-binding proteins (or nucleic acids encoding these proteins) can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34+ cells, include adenovirus Type 35.

Vectors suitable for introduction of transgenes into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells, including T-cells and stem cells of any type. Suitable cell lines for protein expression are known to those of skill in the art and include, but are not limited to COS, CHO (e.g., CHO-S, CHO-K1, CHO-DG44, CHO-DUXB 11), VERO, MDCK, WI38, V79, B14AF28-G3, BHK, HaK, NS0, SP2/0-Agl4, HeLa, HEK293 (e.g., HEK293-F, HEK293-H, HEK293-T), perC6, insect cells such as Spodoptera fugiperda (Sf), and fungal cells such as Saccharomyces, Pichia and Schizosaccharomyces. Progeny, variants and derivatives of these cell lines can also be used.

### Compositions

Pharmaceutically acceptable carriers are determined in part by the particular composition being administered, as well as by the particular method used to administer the composition.

Administration of the compositions of the invention can be carried out via any of the accepted modes of administration of agents for serving similar utilities. The pharmaceutical compositions of the invention may be formulated into preparations in solid, semi solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose. The parenteral preparation can be enclosed in ampoules, disposable syringes or multiple dosevials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, or a protein.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi phasic, or tri phasic systems in order to deliver the active ingredient(s). Delivery of the aerosol includes the necessary container, activators, valves, sub-containers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining the lipid nanoparticles of the invention with sterile, distilled water or other carrier so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compositions of the invention, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions of the invention may also be administered simultaneously with, prior to, or after administration of one or more other therapeutic agents. Such combination therapy includes administration of a single pharmaceutical dosage formulation of a composition of the invention and one or more additional active agents, as well as administration of the composition of the invention and each active agent in its own separate pharmaceutical dosage formulation. For example, a composition of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include C(O) R″ (where R″ is alkyl, aryl or arylalkyl), p methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, (1999), Protective Groups in Organic Synthesis, 3rd Ed., Wiley. As one of skill in the art would appreciate, the protecting group may also be a polymer resin such as a Wang resin, Rink resin or a 2-chlorotrityl-chloride resin.

Furthermore, all lipids which exist in free base or acid form can be converted to their pharmaceutically acceptable salts by treatment with the appropriate inorganic or organic base or acid by methods known to one skilled in the art. Salts of the lipids can be converted to their free base or acid form by standard techniques. The following Reaction Schemes illustrate methods to make lipids of Formula (I), (II), (III) or (IV).

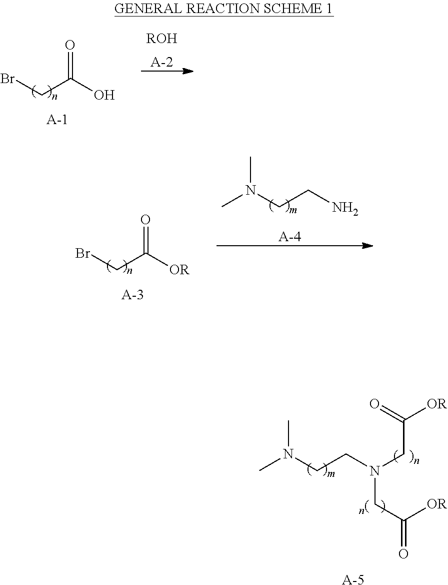

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

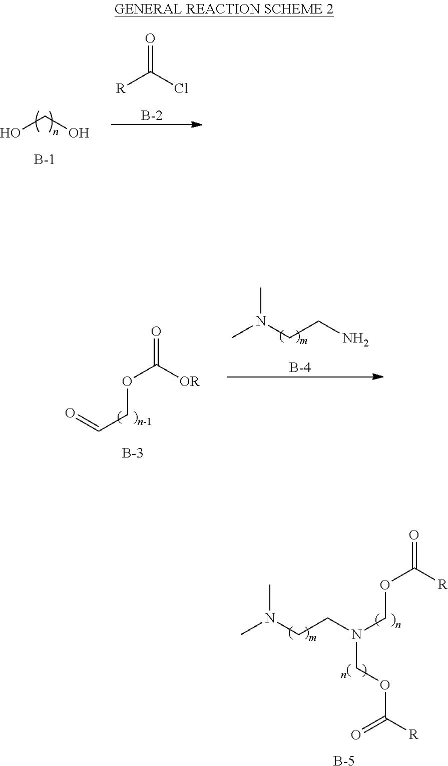

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

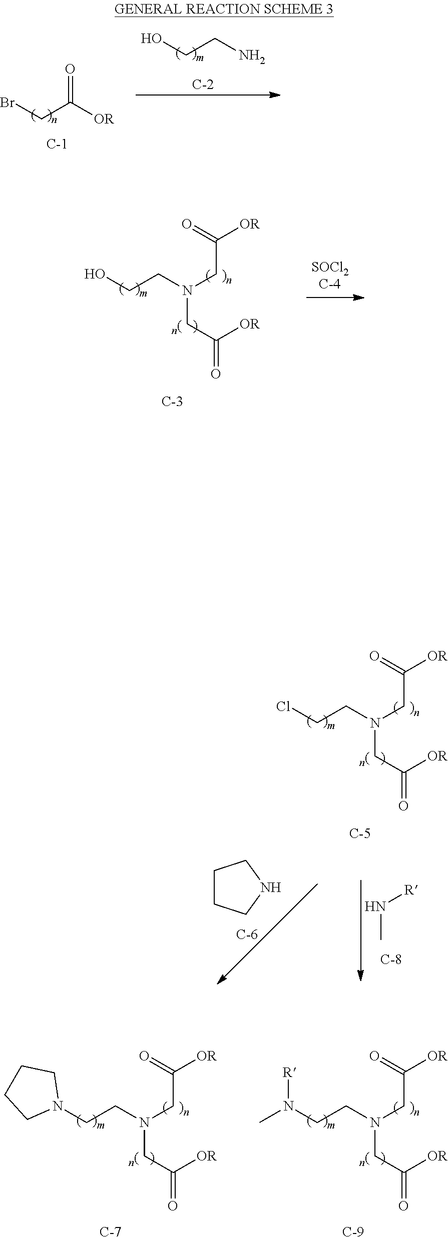

Different embodiments of the lipid of Formula (I) (e.g., compound C-7 or C9) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

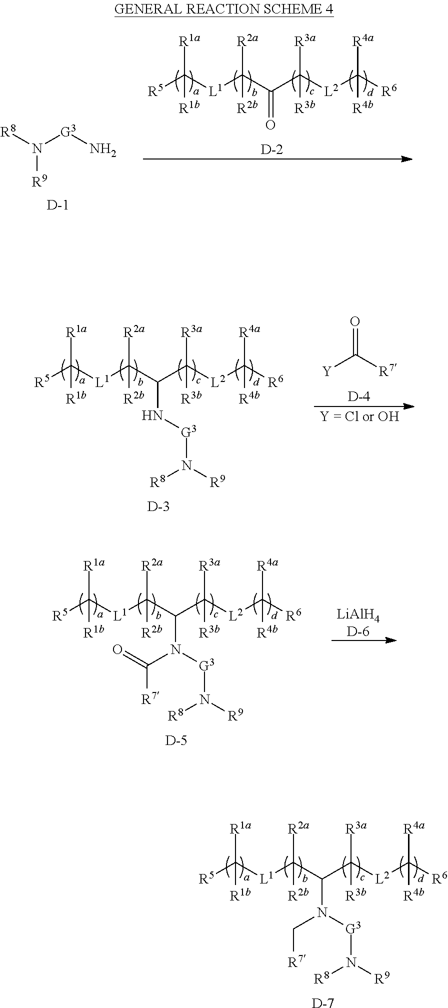

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R^{7′} represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

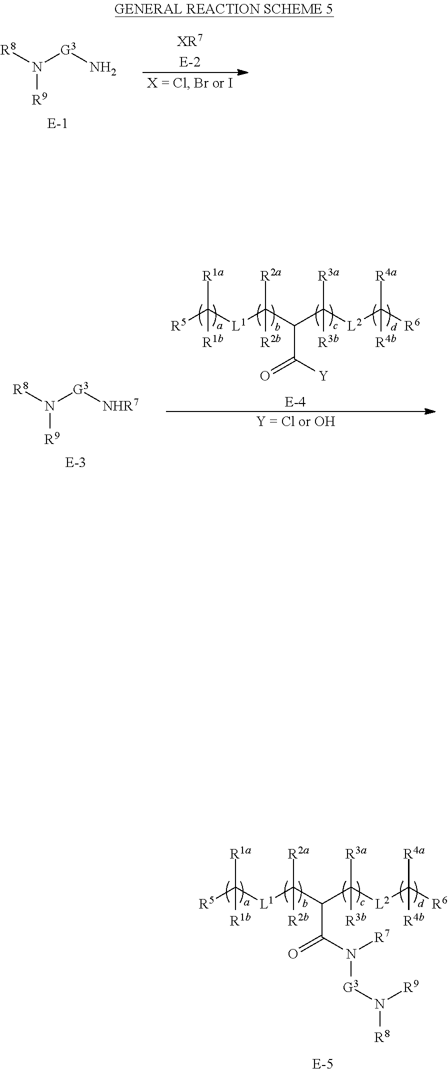

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

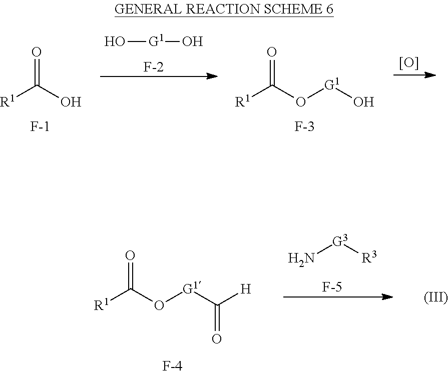

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

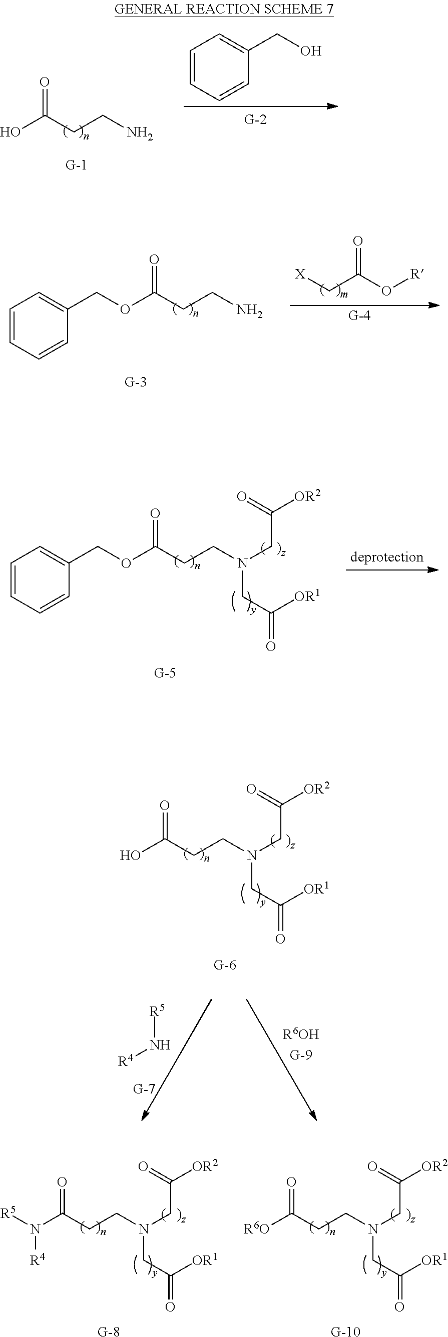

General Reaction Scheme 7 (“Method G”) provides an exemplary method for preparation of compounds of structure (IV). R¹, R², R⁴, R⁵, R⁶, y and z in General Reaction Scheme 7 are as defined herein. R′, X, m and n refer to variables selected such that G-5, G-6, G-8, and G-10 are compounds having a structure (IV). For example, R′ is R¹ or R², X is Br, m is y or z, and n is an integer ranging from 0 to 23. Compounds of structure G-1 are purchased or prepared according to methods known in the art. Amine/acid G-1 is protected with alcohol G-2 (e.g., benzyl alcohol) using suitable conditions and reagents (e.g., p-TSA) to obtain ester/amine G-3. Ester/amine G-3 is coupled with ester G-4 (e.g., using DIPEA) to afford benzyl ester G-5. Compound G-5 is optionally deprotected using appropriate conditions (e.g., Pd/C, H2) to obtain acid G-6. The acid G-6 can be reacted with amine G-7 (e.g., using oxalyl chloride/DMF) to obtain amide G-8, or alternatively, reacted with alcohol G-9 (e.g., using DCC/DMAP) to yield ester G-10. Each of G-5, G-6, G-8, and G-10 are compounds of structure (IV).

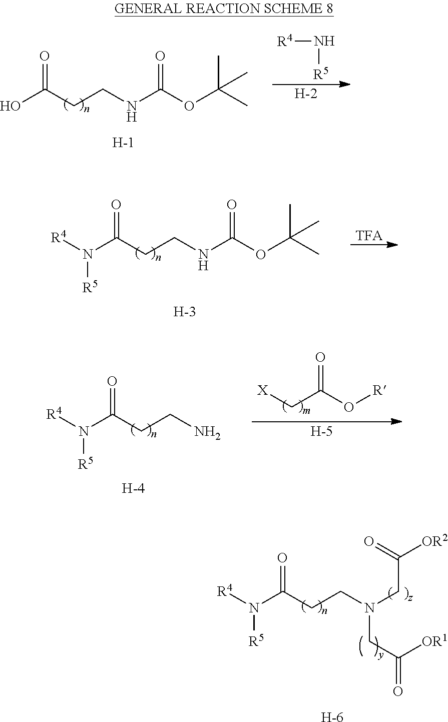

General Reaction Scheme 8 (“Method H”) provides an exemplary method for preparation of compounds of structure (IV). R¹, R², R⁴, R⁵, y and z in General reaction Scheme 8 are as defined herein. R′, X, m and n refer to variables selected such that H-6 is a compound having a structure (IV). For example, R′ is R¹ or R², X is Br, m is y or z, and n is an integer ranging from 0 to 23. Compounds of structure H-1 are purchased or prepared according to methods known in the art. Reaction of protected amine/acid H-1 with amine H-2 is carried out under appropriate coupling conditions (e.g., NHS, DCC) to yield amide H-3. Following a deprotection step using acidic conditions (e.g., TFA), amine H-4 is coupled with ester H-5 under suitable conditions (e.g., DIPEA) to yield H-6, a compound of structure (IV).

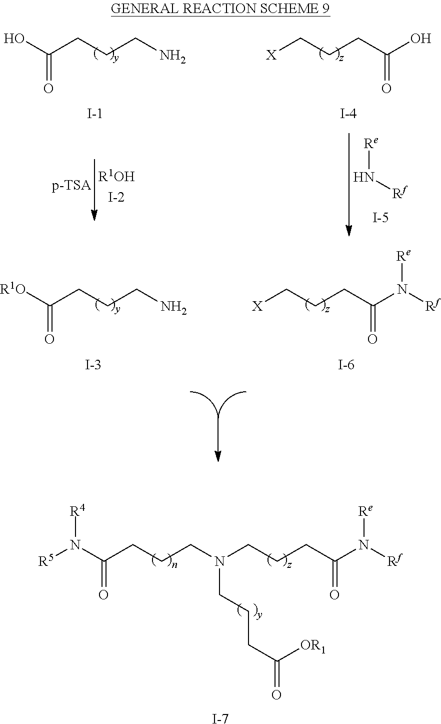

General Reaction Scheme 9 (“Method I”) provides an exemplary method for preparation of compounds of structure (IV). R¹, R⁴, R⁵, R^{e}, R^{f}, y and z in General Reaction Scheme 9 are as defined herein. R′, X, m and n refer to variables selected such that I-7 is a compound having a structure (IV). For example, R′ is R¹ or R², X is Br, m is y or z, and n is an integer ranging from 0 to 23. Compounds of structure I-1, I-2, I-4 and I-5 are purchased or prepared according to methods known in the art. Reaction of amine/acid I-1 with alcohol I-2 is carried out under appropriate coupling conditions (e.g., p-TSA) to yield amine/ester I-3. In parallel, amide I-6 is prepared by coupling acid I-4 with amine I-5 under suitable conditions (e.g., oxalyl chloride/DMF). I-3 and I-6 are combined under basic conditions (e.g., DIPEA) to afford I-7, a compound of structure (IV).

It is understood that one skilled in the art may be able to make these compounds by similar methods or by combining other methods known to one skilled in the art. It is also understood that one skilled in the art would be able to make, in a similar manner as described below, other compounds of structure (I), (II), (III) and (IV) by using the appropriate starting components and modifying the parameters of the synthesis as needed (in addition, see PCT Patent Publication Nos. WO 2015/199952, WO 2017/004143 and WO 2017/075531, which are incorporated herein by reference in their entireties.

Applications

Use of engineered gene therapy in treatment and prevention of disease is expected to be one of the most significant developments in medicine in the coming years. The methods and compositions described herein serve to increase the specificity of these novel tools to ensure that the desired target sites will be the primary place of cleavage. Minimizing or eliminating off-target cleavage will be required to realize the full potential of this technology, for all in vitro, in vivo and ex vivo applications.

Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6^{th} codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Additional exemplary diseases that can be treated by targeted DNA cleavage and/or homologous recombination include acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

Such methods also allow for treatment of infections (viral or bacterial) in a host (e.g., by blocking expression of viral or bacterial receptors, thereby preventing infection and/or spread in a host organism) to treat genetic diseases.

Targeted cleavage of infecting or integrated viral genomes can be used to treat viral infections in a host. Additionally, targeted cleavage of genes encoding receptors for viruses can be used to block expression of such receptors, thereby preventing viral infection and/or viral spread in a host organism. Targeted mutagenesis of genes encoding viral receptors (e.g., the CCR5 and CXCR4 receptors for HIV) can be used to render the receptors unable to bind to virus, thereby preventing new infection and blocking the spread of existing infections. See, U.S. Patent Application No. 2008/015996. Non-limiting examples of viruses or viral receptors that may be targeted include herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae; lentiviruses (e.g., HTLV-I; HTLV-II; HIV-1 (also known as HTLV-III, LAV, ARV, hTLR, etc.) HIV-II); simian immunodeficiency virus (SIV), human papillomavirus (HPV), influenza virus and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses. Receptors for HIV, for example, include CCR-5 and CXCR-4.

As noted above, the compositions and methods described herein can be used for gene modification, gene correction, and gene disruption. Non-limiting examples of gene modification includes homology directed repair (HDR)-based targeted integration; HDR-based gene correction; HDR-based gene modification; HDR-based gene disruption; NHEJ-based gene disruption and/or combinations of HDR, NHEJ, and/or single strand annealing (SSA). Single-Strand Annealing (SSA) refers to the repair of a double strand break between two repeated sequences that occur in the same orientation by resection of the DSB by 5′-3′ exonucleases to expose the 2 complementary regions. The single-strands encoding the 2 direct repeats then anneal to each other, and the annealed intermediate can be processed such that the single-stranded tails (the portion of the single-stranded DNA that is not annealed to any sequence) are be digested away, the gaps filled in by DNA Polymerase, and the DNA ends rejoined. This results in the deletion of sequences located between the direct repeats.

Compositions comprising cleavage domains (e.g., ZFNs, TALENs, CRISPR/Cas systems) and methods described herein can also be used in the treatment of various genetic diseases and/or infectious diseases.

The compositions and methods can also be applied to stem cell based therapies, including but not limited to: correction of somatic cell mutations by short patch gene conversion or targeted integration for monogenic gene therapy; disruption of dominant negative alleles; disruption of genes required for the entry or productive infection of pathogens into cells; enhanced tissue engineering, for example, by modifying gene activity to promote the differentiation or formation of functional tissues; and/or disrupting gene activity to promote the differentiation or formation of functional tissues; blocking or inducing differentiation, for example, by disrupting genes that block differentiation to promote stem cells to differentiate down a specific lineage pathway, targeted insertion of a gene or siRNA expression cassette that can stimulate stem cell differentiation, targeted insertion of a gene or siRNA expression cassette that can block stem cell differentiation and allow better expansion and maintenance of pluripotency, and/or targeted insertion of a reporter gene in frame with an endogenous gene that is a marker of pluripotency or differentiation state that would allow an easy marker to score differentiation state of stem cells and how changes in media, cytokines, growth conditions, expression of genes, expression of siRNA, shRNA or miRNA molecules, exposure to antibodies to cell surface markers, or drugs alter this state; somatic cell nuclear transfer, for example, a patient's own somatic cells can be isolated, the intended target gene modified in the appropriate manner, cell clones generated (and quality controlled to ensure genome safety), and the nuclei from these cells isolated and transferred into unfertilized eggs to generate patient-specific hES cells that could be directly injected or differentiated before engrafting into the patient, thereby reducing or eliminating tissue rejection; universal stem cells by knocking out MHC receptors (e.g., to generate cells of diminished or altogether abolished immunological identity). Cell types for this procedure include but are not limited to, T-cells, B cells, hematopoietic stem cells, and embryonic stem cells. Additionally, induced pluripotent stem cells (iPSC) may be used which would also be generated from a patient's own somatic cells. Therefore, these stem cells or their derivatives (differentiated cell types or tissues) could be potentially engrafted into any person regardless of their origin or histocompatibility.

The compositions and methods can also be used for somatic cell therapy, thereby allowing production of stocks of cells that have been modified to enhance their biological properties. Such cells can be infused into a variety of patients independent of the donor source of the cells and their histocompatibility to the recipient.

In addition to therapeutic applications, the increased specificity provided by the variants described herein when used in engineered nucleases can be used for crop engineering, cell line engineering and the construction of disease models. The obligate heterodimer cleavage half-domains provide a straightforward means for improving nuclease properties.

The engineered cleavage half domains described can also be used in gene modification protocols requiring simultaneous cleavage at multiple targets either to delete the intervening region or to alter two specific loci at once. Cleavage at two targets would require cellular expression of four ZFNs or TALENs, which could yield potentially ten different active ZFN or TALEN combinations. For such applications, substitution of these novel variants for the wild-type nuclease domain would eliminate the activity of the undesired combinations and reduce chances of off-target cleavage. If cleavage at a certain desired DNA target requires the activity of the nuclease pair A+B, and simultaneous cleavage at a second desired DNA target requires the activity of the nuclease pair X+Y, then use of the mutations described herein can prevent the pairings of A with A, A with X, A with Y and so on. Thus, these FokI mutations decrease non-specific cleavage activity as a result of “illegitimate” pair formation and allow the generation of more efficient orthogonal mutant pairs of nucleases (see U.S. Patent Publication Nos. 20080131962 and 20090305346).

All patents, patent applications and publications mentioned herein are hereby incorporated by reference in their entireties.

Although disclosure has been provided in some detail by way of illustration and example for the purposes of clarity and understanding, it will be apparent to those of skill in the art that various changes and modifications can be practiced without departing from the spirit or scope of the disclosure. Accordingly, the foregoing disclosure and following examples should not be construed as limiting.

### EXAMPLES

### Example 1: Preparation of ZFNs

ZFNs targeted to sites in the mouse albumin, TTR and PCSK9 genes were designed and incorporated into plasmids vectors essentially as described in Urnov et al. (2005) Nature 435(7042):646-651, Perez et al (2008) Nature Biotechnology 26(7): 808-816, and U.S. Pat. Nos. 9,394,545 and 9,150,847; PCT Patent Application No: PCT/US2016/032049 and U.S. Publication Nos. 20170211075 and 20170173080. The ZFNs were tested and all were found to be active. The ZFNs used are shown below in Table 4, and the sequences that are targeted are shown in Table 5:

TABLE 4
ZFN designs

| SBS # | Design | | | | | | Domain |
|---|---|---|---|---|---|---|---|
| (gene) | F1 | F2 | F3 | F4 | F5 | F6 | linker |
| 30724 | TSGSLTR | RSDALST | QSATRTK | TSGHLSR | QSGNLAR | N/A | 5,6 |
| (mALB) | (SEQ ID | (SEQ ID | (SEQ ID | (SEQ ID | (SEQ ID | | |
| | NO: 1) | NO: 2) | NO: 3) | NO: 4) | NO: 5) | | |
| 30725 | RSDHLSA | TKSNRTK | DRSNLSR | WRSSLRA | DSSDRKK | N/A | 5,6 |
| (mALB) | (SEQ ID | (SEQ ID | (SEQ ID | (SEQ ID | (SEQ ID | | |
| | NO: 6) | NO: 7) | NO: 8) | NO: 9) | NO: 10) | | |
| 48641 | TSGSLTR | RSDALST | QSATRTK | LRHHLTR | QAGQRRV | N/A | 5,6 |
| (mALB) | (SEQ ID | (SEQ ID | (SEQ ID | (SEQ ID | (SEQ ID | | |
| | NO: 1) | NO: 2) | NO: 3) | NO: 11) | NO: 12) | | |
| 31523 | RSDNLSE | QSGNLAR | DRSNLSR | WRSSLRA | DSSDRKK | N/A | 5,6 |
| (mALB) | (SEQ ID | (SEQ ID | (SEQ ID | (SEQ ID | (SEQ ID | | |
| | NO: 13) | NO: 5) | NO: 8) | NO: 9) | NO: 10) | | |
| 59771 | QSSNLAR | QSGHLSR | QSSDLSR | TSGHLSR | RSDNLSE | ASKTRKN | N7a |
| (mTTR) | (SEQ ID | (SEQ ID | (SEQ ID | (SEQ ID | (SEQ ID | (SEQ ID | |
| | NO: 14) | NO: 15) | NO: 16) | NO: 4) | NO: 13) | NO: 17) | |
| 59790 | QSGHLAR | QLTHLNS | SKLYLNN | DRSNLTR | YRWLRNS | DRSNLTR | N7a |
| (mTTR) | (SEQ ID | (SEQ ID | (SEQ ID | (SEQ ID | (SEQ ID | (SEQ ID | |
| | NO: 18) | NO: 19) | NO: 20) | NO: 21) | NO: 22) | NO: 21) | |
| 58780 | HGQTLNE | QSGNLAR | RSDNLSE | SKQYLIK | DRSHLTR | QSGHLSR | N7a |
| (mPCSK9) | (SEQ ID | (SEQ ID | (SEQ ID | (SEQ ID | (SEQ ID | (SEQ ID | |
| | NO: 23) | NO: 5) | NO: 13) | NO: 24) | NO: 25) | NO: 15) | |
| 61748 | DRSNLSR | QSGHLSR | DRSHLSR | TSGNLTR | QSSDLSR | TSGHLSR | L0 |
| (mPCSK9) | (SEQ ID | (SEQ ID | (SEQ ID | (SEQ ID | (SEQ ID | (SEQ ID | |
| | NO: 8) | NO: 15) | NO: 26) | NO: 27) | NO: 16) | NO: 4) | |
| 48652 | LRHHLTR | LRHNLRA | DRSHLAR | TSGHLSR | QSGNLAR | N/A | 5,6 |
| (mALB) | (SEQ ID | (SEQ ID | (SEQ ID | (SEQ ID | (SEQ ID | | |
| | NO: 11) | NO: 28) | NO: 29) | NO: 4) | NO: 5) | | |
| 31527 | RSDHLSE | QSGNLAR | DRSNLSR | WRSSLRA | DSSDRKK | N/A | 5,6 |
| (mALB) | (SEQ ID | (SEQ ID | (SEQ ID | (SEQ ID | (SEQ ID | | |
| | NO: 30) | NO: 5) | NO: 8) | NO: 9) | NO: 10) | | |

TABLE 5
Target sequences

| SBS number | TaxgetSequence (5′→3′) |
|---|---|
| 30724 | ctGAAGGTgGCAATGGTTcctctctgct (SEQ ID NO: 31) |
| 30725 | ttTCCTGTAACGATCGGgaactggcatc (SEQ ID NO: 32) |
| 48641 | ctGAAGGTgGCAATGGTTcctctctgct (SEQ ID NO: 31) |
| 31523 | ttTCCTGTAACGATCGGgaactggcatc (SEQ ID NO: 32) |
| 59771/69121/69052 | gtGCCCAGGGTGCTGGAGAAtccaaatg (SEQ ID NO: 33) |
| 59790/69128/69102 | agGACTTTGACCATcAGAGGAcatttgg (SEQ ID NO: 34) |
| 58780 | ctGGAGGCTGCCAGGAACCTacattgtg (SEQ ID NO: 35) |
| 61748 | gtGGTGCTGATGGAGGAGACccagaggc (SEQ ID NO: 36) |
| 48652 | ctGAAGGTGGCAATGGTtcctctctgct (SEQ ID NO: 31) |
| 31527 | ttTCCTGTAACGATCGGgaactggcatc (SEQ ID NO: 32) |

Some ZFNs were further modified to remove potential phosphate contacting amino acids in the ZFP backbone or FokI domain. These residues have the potential to interact with the phophates on the DNA backbone, leading to non-specific cleavage (described in U.S. application Ser. No. 15/697,917). Table 6 shows parent and derivative ZFNs where the ZFP backbone has been mutated at the indicated locations to remove potential non-specific phosphate contacts.

TABLE 6
Optimized ZFN designs

| | Design | | | | | | | FokI |
|---|---|---|---|---|---|---|---|---|
| SBS# | F1 | F2 | F3 | F4 | F5 | F6 | Linker | mutants |
| SBS# | QSSNLAR | QSGHLSR | QSSDLSR | TSGHLSR | RSDNLSE | ASKTREN | N7a | ELD |
| 59771 | (SEQ ID | (SEQ ID | (SEQ ID | (SEQ ID | (SEQ ID | (SEQ ID | | |
| [Parent] | NO: 14) | NO: 15) | NO: 16) | NO: 4) | NO: 13) | NO: 17) | | |
| SBS+190 | QSSNLAR | QSGHLSR | QSSDLSR | TSGHLSR | RSDNLSE | ASKTREN | N7a | ELD |
| 69121 | (SEQ ID | (SEQ ID | (SEQ ID | (SEQ ID | (SEQ ID | (SEQ ID | | |
| | NO: 14) | NO: 15) | NO: 16) | NO: 4) | NO: 13) | NO: 17) | | |
| 69121 | none | none | none | none | none | none | | D421S, |
| mutants | | | | | | | | Q531R |
| SBS# | QSSNLAR | QSGHLSR | QSSDLSR | TSGHLSR | RSDNLSE | ASKTREN | N7a | ELD |
| 69052 | (SEQ ID | (SEQ ID | (SEQ ID | (SEQ ID | (SEQ ID | (SEQ ID | | |
| | NO: 14) | NO: 15) | NO: 16) | NO: 4) | NO: 13) | NO: 17) | | |
| 69052 | none | Qm5 | none | Qm5 | none | Qm5 | | S418P |
| mutants | | | | | | | | |
| SBS# | QSGHLAR | QLTHLNS | SKLYLNN | DRSNLTR | YRWLRNS | DRSNLTR | N7a | KKR |
| 59790 | (SEQ ID | (SEQ ID | (SEQ ID | (SEQ ID | (SEQ ID | (SEQ ID | | |
| [parent] | NO: 18) | NO: 19) | NO: 20) | NO: 21) | NO: 22) | NO: 21) | | |
| SBS# | QSGHLAR | QLTHLNS | SKLYLNN | DRSNLTR | YRWLRNS | DRSNLTR | N7a | KKR |
| 69128 | (SEQ ID | (SEQ ID | (SEQ ID | (SEQ ID | (SEQ ID | (SEQ ID | | |
| | NO: 18) | NO: 19) | NO: 20) | NO: 21) | NO: 22) | NO: 21) | | |
| 69128 | none | none | none | none | none | none | | D421S, |
| mutants | | | | | | | | Q481H |
| SBS# | QSGHLAR | QLTHLNS | SKLYLNN | DRSNLTR | YRWLRNS | DRSNLTR | N7a | |
| 69102 | (SEQ ID | (SEQ ID | (SEQ ID | (SEQ ID | (SEQ ID | (SEQ ID | | |
| | NO: 18) | NO: 19) | NO: 20) | NO: 21) | NO: 22) | NO: 21) | | |
| 69102 | none | Qm5 | none | Qm5 | none | Qm5 | | S418P |
| mutants | | | | | | | | |

ZFNs targeting intron 1 of the murine ALB gene, exon 2 of the murine PCSK9 gene, and exon 2 of the murine TTR gene (all shown above in Tables 4 and 5) were subcloned into individual vectors (pVAV-GEM) containing a T7 RNA polymerase promoter, a 5′ UTR containing a sequence derived from the Xenopus beta-globin gene, a 3′UTR containing a dual cassette of a sequence derived from the HBB gene, and a 64base pair polyA tract. The sequences of the Xenopus 5′ UTR, the HBB 3′ UTR, and the WPRE 3′ UTR were as follows:

Xenopus beta-globin 5′UTR [Falcone et al. (1991) Molecular and Cellular Biology 11(5):2656-2664]:

(SEQ ID NO: 37)
5′ TGCTTGTTCTTTTTGCAGAAGCTCAGAATAAACGCTCAACTTTGGCA
GATC
Dual HBB 3′ UTR [Russell et al. (1996) Blood 87:
5314-5323]:
(SEQ ID NO: 38)
5′CTAGAAGCTCGCTTTCTTGCTGTCCAATTTCTATTAAAGGTTCCTTTG
TTCCCTAAGTCCAACTACTAAACTGGGGGATATTATGAAGGGCCTTGAGC
ATCTGGATTCTGCCTAATAAAAAACATTTATTTTCATTGCTGCGCTAGAA
GCTCGCTTTCTTGCTGTCCAATTTCTATTAAAGGTTCCTTTGTTCCCTAA
GTCCAACTACTAAACTGGGGGATATTATGAAGGGCCTTGAGCATCTGGAT
TCTGCCTAATAAAAAACATTTATTTTCATTGCTGCG
WPRE 3′ UTR (see U.S. Pat. Application Ser. No.
15/141,333):
(SEQ ID NO: 39)
5′AATCAACCTCTGGATTACAAAATTTGTGAAAGATTGACTGGTATTCTT
AACTATGTTGCTCCTTTTACGCTATGTGGATACGCTGCTTTAATGCCTTT
GTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAA
CGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACA
GGGGCTCGGCTGTTGGGCACTGACAATTCCGTGGTGTTGTCGGGGAAGCT
GACGTCCTTTCCATGGCTGCTCGCCTGTGTTGCCACCTGGATTCTGCGCG
GGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCG
CCCTCAGACGAGTCGGATCTCCCTTTGGGCCGCCTCCCCGCCTG.

### Example 2: Methods

mRNA Synthesis:

Messenger RNA (mRNA) was produced of the SpeI-linearized ZFN constructs using in vitro transcription (IVT) at Trilink Biotechnologies with either unmodified residues or a fraction of modified nucleosides (2 thiouridine (“2tU”), and 5 methylcytosine (“5mC”)). mRNA was capped either co-transcriptionally with an anti-reverse cap analog (ARCA) cap, enzymatically post-IVT using the Vaccinia virus capping enzyme along with the mRNA Cap 2′-O-Methyltransferase enzyme to produce “Cap1” mRNA, or chemically to produce “Cap1” (CleanCap). mRNA was purified through a silica bead column (for example see Bowman et al (2012) Methods v. 941 Conn G. L. (ed), New York, N.Y. Humana Press), and then packaged (silica-purified) or subsequently ran through an HPLC column and fractionated to remove double stranded RNA species and then packaged (HPLC-purified, for example see Kariko et al (2011) Nucl Acid Res 39:e142; Weissman et al (2013) Synthetic Messenger RNA and Cell Metabolism Modulation in Methods in Molecular Biology 969, Rabinovich, P. H. Ed).

LNPs:

LNPs were prepared according to the general procedures described in WO 2015/199952, WO 2017/004143 and WO 2017/075531, which are incorporated herein by reference in its entireties. Briefly, Cationic lipid, DSPC, cholesterol and a PEG-lipid of Formula (IVa) were solubilized in ethanol at a molar ratio of approximately 50:10:38.5:1.5 or 47.5:10:40.8:1.7. Lipid nanoparticles (LNP) were prepared at a ratio of mRNA to Total Lipid of 0.03-0.04 w/w. The ZFN mRNA was diluted to 0.05 to 0.2 mg/mL in 10 to 50 mM citrate buffer, pH 4. Syringe pumps were used to mix the ethanolic lipid solution with the mRNA aqueous solution at a ratio of about 1:5 to 1:3 (vol/vol) with total flow rates above 15 ml/min. The ethanol was then removed and the external buffer replaced with PBS by dialysis. Finally, the lipid nanoparticles were filtered through a 0.2 μm pore sterile filter. Lipid nanoparticle particle diameter size was 60-90 nm as determined by quasi-elastic light scattering using a Malvern Zetasizer Nano (Malvern, UK). Formulations are used as prepared above within about 24 hours, or optionally sucrose is added at a final concentration of 300 mM as a cryprotectant for longer term storage stability.

Unique formulation numbers (e.g., I-6, I-5, and II-9) refer to unique cationic lipids, while the other 3 lipid components remain constant. Synthesis of representative lipids are described herein below and in WO 2015/199952, WO 2017/004143 and WO 2017/075531, each incorporated herein by reference.

In Vitro Transduction:

To assess the activity of LNPs containing the ZFN mRNA, LNPs were introduced into media containing Hepa1-6 cells in suspension and cells were allowed to adhere, uptake LNPs, and grow for 3 days. Transduced cells were then harvested for genomic DNA (gDNA) using Qiagen spin columns.

In Vivo Transduction:

8-10 week old C57BL6 purchased from Charles River were injected intravenously through the tail vein with 200 uL of an aqueous solution containing diluted LNPs or a mixture of diluted LNPs and AAV donor encoding a human IDS or human FIX transgene containing homology arms flanking the ZFN cut site and a splice acceptor just upstream of the coding sequence of the transgene. Some animals were injected with 5 mg/kg dexamethasone intraperitoneally 30 minutes prior to LNP dosing. Animals were then sacrificed 7 days post LNP dosing or re-dosed with LNPs at either a 7 or 14 day interval before subsequent sacrifice and harvesting of liver tissue. Livers were snap frozen and a small portion of both the left and right lobes were dissected and harvested for gDNA using a FastPrep-24 Homogenizer (MP Biomedicals), Lysis Matrix D solution (MP Biomedicals), and a MasterPure DNA Purification kit (Epicentre).

Indel Analysis (Nuclease Activity):

Primers were designed to amplify approximately 200 bp of total genomic DNA sequence containing the ZFN cut site. Amplicons were then ran on either a Miseq or Nextseq (Illumina) and insertions and deletions (indels) from the wildtype genomic sequence were quantified.

IDS Enzyme Activity Assay:

At animal sacrifice, mouse blood was collected into tubes containing sodium citrate and the cell fraction was removed to yield mouse plasma. Plasma was then diluted 1:100 in water and incubated with Iduronate-2-sulfatase (IDS) substrate (4-Methylumbelliferyl-α-iduronate 2-sulphate) for 4 hours at 37° C. A 0.4M sodium phosphate solution was then added to halt the reaction. Recombinant human iduronidase (IDUA) was then added and the solution was then incubated for 24 hours at 37° C. IDS substrate which has been successfully cleaved will yield a fluorescent product which is then measured on a fluorescent plate reader at 365 nm excitation/450 nm emission.

hFIX ELISA:

Mouse plasma was diluted 1:100 in PBS and ran for hFIX protein levels on a sandwich ELISA kit (Affinity Biologicals). Absorbance was read at 450 nm using a microplate reader.

mPCSK9 ELISA:

Mouse plasma was diluted 1:100 in PBS and ran for mPCSK9 protein levels on a sandwich ELISA kit (Boster Biological Technology). Absorbance was read at 450 nm using a microplate reader.

mTTR ELISA:

Mouse plasma was diluted 1:10,000 in PBS with 0.5% BSA and ran for mTTR protein levels on a sandwich ELISA kit (Cusabio). Absorbance was read at 450 nm using a microplate reader.

### Preparation of I-5:

Compound I-5 was prepared according to method B as follows:

A solution of hexan-1,6-diol (10 g) in methylene chloride (40 mL) and tetrahydrofuran (20 mL) was treated with 2-hexyldecanoyl chloride (10 g) and triethylamine (10 mL). The solution was stirred for an hour and the solvent removed. The reaction mixture was suspended in hexane, filtered and the filtrate washed with water. The solvent was removed and the residue passed down a silica gel (50 g) column using hexane, followed by methylene chloride, as the eluent, yielding 6-(2′-hexyldecanoyloxy)hexan-1-ol as an oil (7.4 g).

The purified product (7.4 g) was dissolved in methylene chloride (50 mL) and treated with pyridinum chlorochromate (5.2 g) for two hours. Diethyl ether (200 mL) as added and the supernatant filtered through a silica gel bed. The solvent was removed from the filtrate and resultant oil passed down a silica gel (50 g) column using a ethyl acetate/hexane (0-5%) gradient. 6-(2′-hexyldecanoyloxy)dodecanal (5.4 g) was recovered as an oil.

A solution of the product (4.9 g), acetic acid (0.33 g) and 2-N,N-dimethylaminoethylamine (0.40 g) in methylene chloride (20 mL) was treated with sodium triacetoxyborohydride (2.1 g) for two hours. The solution was washed with aqueous sodium hydroxide. The organic phase was dried over anhydrous magnesium sulfate, filtered and the solvent removed. The residue was passed down a silica gel (50 g) column using a methanol/methylene chloride (0-8%) gradient to yield the desired product (1.4 g) as a colorless oil.

### Preparation of I-6:

Compound I-6 was prepared according to method B as follows:

A solution of nonan-1,9-diol (12.6 g) in methylene chloride (80 mL) was treated with 2-hexyldecanoic acid (10.0 g), DCC (8.7 g) and DMAP (5.7 g). The solution was stirred for two hours. The reaction mixture was filtered and the solvent removed. The residue was dissolved in warmed hexane (250 mL) and allowed to crystallize. The solution was filtered and the solvent removed. The residue was dissolved in methylene chloride and washed with dilute hydrochloric acid. The organic fraction was dried over anhydrous magnesium sulfate, filtered and the solvent removed. The residue was passed down a silica gel column (75 g) using 0-12% ethyl acetate/hexane as the eluent, yielding 9-(2′-hexyldecanoyloxy)nonan-1-ol (9.5 g) as an oil.

The product was dissolved in methylene chloride (60 mL) and treated with pyridinum chlorochromate (6.4 g) for two hours. Diethyl ether (200 mL) as added and the supernatant filtered through a silica gel bed. The solvent was removed from the filtrate and resultant oil passed down a silica gel (75 g) column using a ethyl acetate/hexane (0-12%) gradient, yielding 9-(2′-ethylhexanoyloxy)nonanal (6.1 g) as an oil.

A solution of the crude product (6.1 g), acetic acid (0.34 g) and 2-N,N-dimethylaminoethylamine (0.46 g) in methylene chloride (20 mL) was treated with sodium triacetoxyborohydride (2.9 g) for two hours. The solution was diluted with methylene chloride washed with aqueous sodium hydroxide, followed by water. The organic phase was dried over anhydrous magnesium sulfate, filtered and the solvent removed. The residue was passed down a silica gel (75 g) column using a methanol/methylene chloride (0-8%) gradient, followed by a second column (20 g) using a methylene chloride/acetic acid/methanol gradient. The purified fractions were dissolved in methylene chloride, washed with dilute aqueous sodium hydroxide solution, dried over anhydrous magnesium sulfate, filtered and the solvent removed, to yield the desired product (1.6 g) as a colorless oil.

### Preparation of II-9:

Compound II-9 was prepared according to method D as follows:

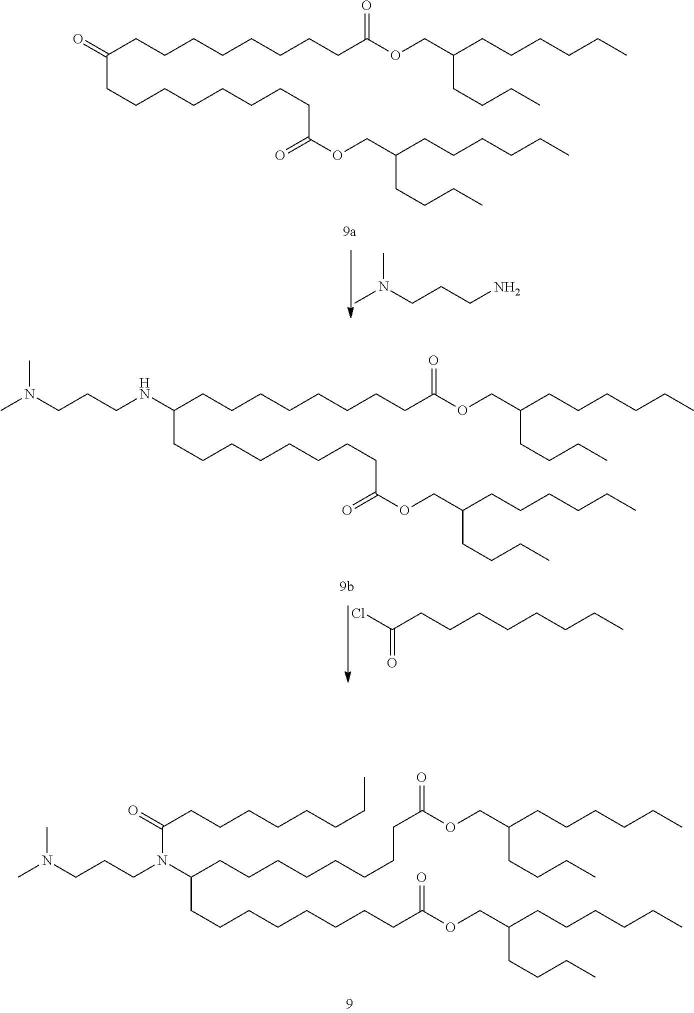

### Step 1:

3-dimethylamine-1-propylamine (1 eq. 1.3 mmol, 133 mg, 163 uL; MW102.18, d 0.812) and the ketone 9a (1 eq., 0.885 g, 1.3 mmol) were mixed in DCE (8 mL) and then treated with sodium triacetoxyborohydride (1.4 eq., 1.82 mmol, 386 mg; MW211.94) and AcOH (1 eq., 1.3 mmol, 78 mg, 74 uL, MW 60.05, d 1.06). The mixture was stirred at RT under an Ar atmosphere for 2 days. The reaction mixture was diluted with hexanes-EtOAc (9:1) and quenched by adding 0.1 N NaOH (20 mL). The organic phase was separated, washed with sat NaHCO3, brine, dried over sodium sulfate, decanted and concentrated to give the desired product 9b as a slightly yellow cloudy oil (1.07 g, 1.398 mmol).

### Step 2:

A solution of nonanoyl chloride (1.3 eq., 1.27 mmol, 225 mg) in benzene (10 mL) was added via syringe to a solution of the compound 9b from step 1 (0.75 g, 0.98 mmol) and triethylamine (5 eq, 4.90 mmol, 0.68 mL) and DMAP (20 mg) in benzene (10 mL) at RT in 10 min. After addition, the mixture was stirred at RT overnight. Methanol (5.5 mL) was added to remove excess acyl chloride. After 3 h, the mixture was filtered through a pad of silica gel (1.2 cm). Concentration gave a colorless oil (0.70 g).

The crude product (0.70 g) was purified by flash dry column chromatography on silica gel (0 to 4% MeOH in chloroform). This yielded 457 mg of colorless oil, 0.50 mmol, 51%. 1HNMR (400 MHz, CDCl3) δ: 4.54-4.36 (very br., estimated 0.3H, due to slow isomerization about amide bond), 3.977, 3.973 (two sets of doublets, 5.8 Hz, 4H), 3.63 (quintet-like, 6.8 Hz, 0.7H), 3.14-3.09 (m, 2H), 2.33-2.25 (m, 8H), 2.23, 2.22 (two sets of singlet, 6H), 1.76-1.56 (m, 10H), 1.49-1.39 (m, 4H), 1.37-1.11 (62H), 0.92-0.86 (m, 15H).

### Preparation of II-11:

Compound II-11 was prepared according to the general procedure D to yield 239 mg of colorless oil, 0.26 mmol, total yield 52% for 2 steps. ¹HNMR (400 MHz, CDCl3) δ: 4.87 (quintet-like, 6.3 Hz, 2H), 4.54-4.36 (very br., estimated 0.3H, due to slow isomerization about amide bond), 3.63 (quintet-like, 6.8 Hz, 0.7H), 3.14-3.09 (m, 2H), 2.33-2.25 (m, 8H), 2.23, 2.22 (two sets of singlet, 6H), 1.76-1.56 (m, 8H), 1.55-1.39 (m, 12H), 1.37-1.11 (62H), 0.92-0.86 (m, 15H).

### Preparation of II-36

Compound II-36 was prepared according to the general procedure D to yield 234 mg of colorless oil, 0.25 mmol, total yield 34% for 2 steps. ¹HNMR (400 MHz, CDCl3) δ: 4.54-4.36 (br., estimated 0.3H, due to slow isomerization about amide bond), 3.977, 3.973 (two sets of doublets, 5.8 Hz, 4H), 3.63 (quintet-like, 6.8 Hz, 0.7H), 3.17-3.10 (m, 2H), 2.53-2.43 (m, 6H), 2.34-2.26 (m, 6H), 1.83-1.71 (m, 6H), 1.70-1.57 (m, 8H), 1.49-1.38 (m, 4H), 1.37-1.11 (62H), 0.92-0.86 (m, 15H).

### Preparation of III-25

A solution of nonan-1,9-diol (12.0 g) in methylene chloride (150 mL) was treated with 2-butyloctanoic acid (5.0 g), DCC (7.7 g) and DMAP (4.5 g). The solution was stirred overnight. The reaction mixture was filtered and the solvent removed. The residue was suspended in hexane and filtered. The filtrate was washed with dilute hydrochloric acid. The organic phase was dried over anhydrous magnesium sulfate, filtered through a silica gel bed, and the solvent removed. The crude product was passed down a silica gel column using a methanol/methylene chloride (0-4%) gradient, to produce 9-(2′-butyloctanoyloxy)nonan-1-ol (6 g) as an oil.

The 9-(2′-butyloctanoyloxy)nonan-1-ol was dissolved in methylene chloride (100 mL) and treated with pyridinium chlorochromate (3.8 g) overnight. Hexane (300 mL) was added and the supernatant filtered through a silica gel bed. The solvent was removed from the filtrate and resultant oil dissolved in hexane. The suspension was filtered through a silica gel bed and the solvent removed, yielding 9-(2′-butyloctanoyloxy)nonan-1-al (3.1 g) was obtained as a colorless oil.

A solution of 9-(2′-butyloctanoyloxy)nonan-1-al (2.6 g), acetic acid (0.20 g) and 4-aminobutan-1-ol (0.26 g) in methylene chloride (50 mL) was treated with sodium triacetoxyborohydride (1.42 g) overnight. The solution was washed with aqueous sodium hydrogen carbonate solution. The organic phase was dried over anhydrous magnesium sulfate, filtered and the solvent removed. The residue was passed down a silica gel column using a using an acetic acid/methanol/methylene chloride (2-0/0-12/98-88%) gradient. Pure fractions were washed with aqueous sodium bicarbonate solution, yielding compound III-25 as a colorless oil (0.82 g).

### Preparation of 111-45:

III-45 was prepared as follows. A solution of 9-(2′-butyloctanoyloxy)nonan-1-al (2.6 g), acetic acid (0.17 g) and 3-aminopropan-1-ol (0.21 g) in methylene chloride (50 mL) was treated with sodium triacetoxyborohydride (1.34 g) overnight. The solution was washed with aqueous sodium hydrogen carbonate solution. The organic phase was dried over anhydrous magnesium sulfate, filtered and the solvent removed. The residue was passed down a silica gel column using a using an acetic acid/methanol/methylene chloride (2-0/0-8/98-96%) gradient. Pure fractions were washed with aqueous sodium bicarbonate solution, yielding compound 45 as a colorless oil (1.1 g).

### Preparation of 111-49:

To a solution of 2-butyloctyl 8-bromooctanoate (2 eq, 1.877 g, 4.8 mmol) in 20 ml of anhydrous THF, were added 4-amino-1-butanol (1 eq. 2.4 mmol, 214 mg, 221 ul), potassium carbonate (2 eq, 4.8 mmol, 664 mg), cesium carbonate (0.3 eq, 0.72 mmol, 234 mg) and sodium iodide (ca 5 mg). The mixture in a pressure round-bottom flask was heated (oil bath, 80° C.) for 6 days. The reaction mixture was cooled and concentrated. The residue was taken up in a mixture of hexane and ethyl acetate (ca 5:1), washed with water, brine, dried over sodium sulfate, filtered and concentrated. The residue was purified flash column chromatography on silica gel (methanol in chloroform, 1 to 4%). This gave compound 49 as a colorless oil (857 mg, 1.21 mmol, 50%). ¹HNMR (400 MHz, CDCl3) δ: 6.55 (br. s, 1H), 3.97 (d, 5.8 Hz, 4H), 3.55 (not well resolved triplet, 2H), 2.45-2.40 (m. 6H), 2.30 (t, 7.5 Hz, 4H), 1.71-1.58 (m, 10H), 1.51-1.42 (m, 4H), 1.39-1.19 (m, 44H), 0.93-0.87 (m, 12H).

Preparation of IV-12: Compound IV-12 was prepared according to the following reaction scheme:

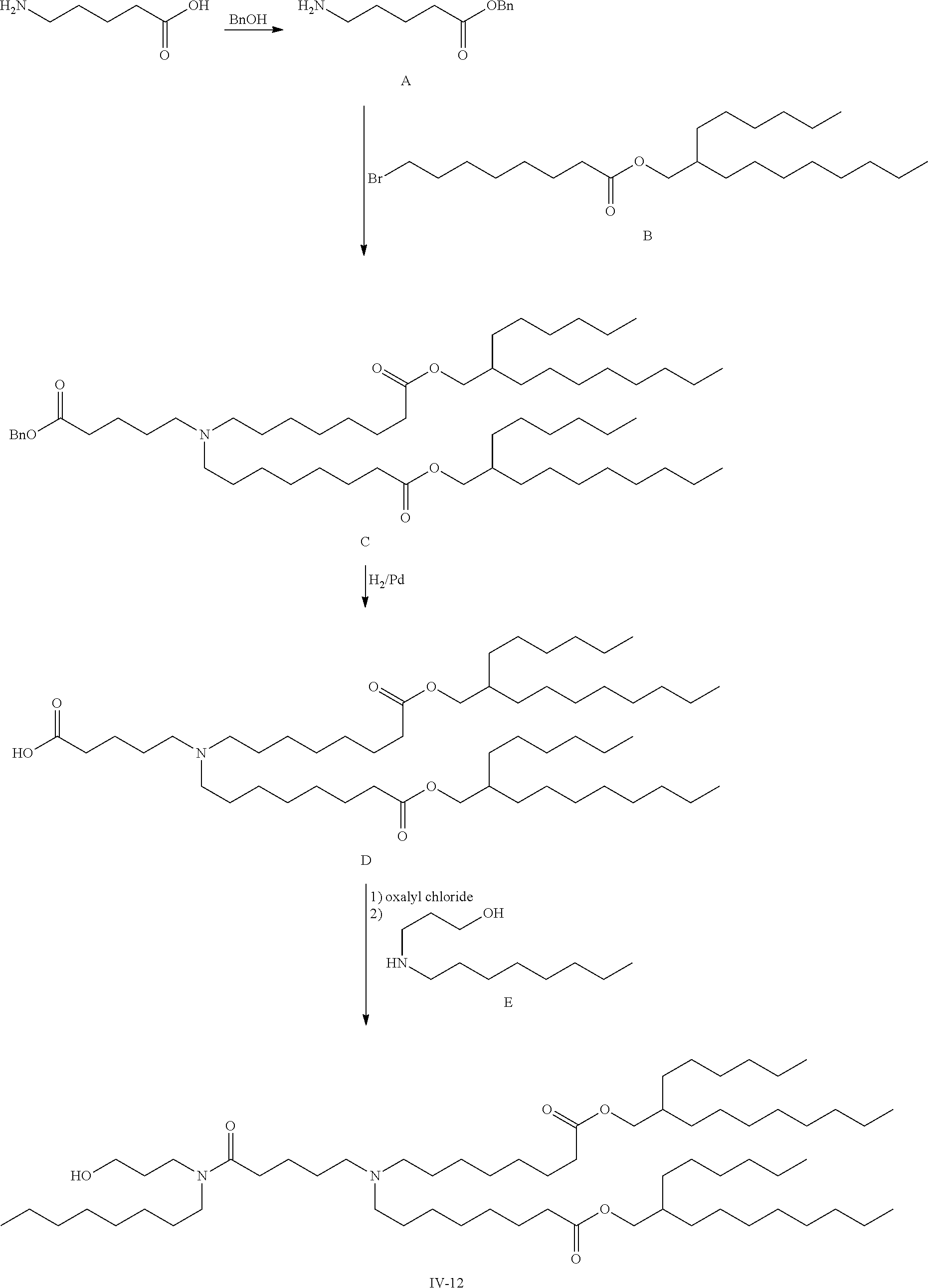

### Synthesis of A:

A mixture of 5-aminovaleric acid (1 eq. 2.9 g, 24.8 mmol), benzyl alcohol (2.3 eq, 58 mmol, 6.26 g, 6 mL), toluene (70 mL) and p-toluenesulfonic acid monohydrate (1.1 eq, 23.4 mmol, 4.45 g) was heated to reflux for 20 hours under Dean-Stark conditions. The mixture was cooled to RT. The solid was collected by filtration and was washed with toluene (20 mL×2) and diethyl ether (20 mL). The desired product (as t-TsOH salt) was obtained as a white solid (7.503 g, 19.8 mmol, 80%).

### Synthesis of C:

A mixture of A (1 eq, 5.59 mmol, 2.5 g), B (salt form, 1.4 eq., 3 g, 7.9 mmol, MW 379.47), N,N-diisopropylethylamine (3.5 equiv., 27.67 mmol, 3.58 g, 4.82 mL) and anhydrous acetonitrile (20 mL) was heated for 16 h in a sealed pressure flask (oil bath 83 C). The crude product was purified by column chromatography on silica gel (hexane-EtOAc-Et3N, from 95:5:0 to 75:25:1). This gave the desired product C as a yellow oil, 912 mg, 0.97 mmol, 35%).

### Synthesis of D:

To a solution of C (912 mg, 0.97 mmol) in EtOH-EtOAc (1:10 mL) was added 10% Pd/C (25 mg), and the mixture was stirred under hydrogen for 16 h. The reaction mixture was filtered through a pad of Celite© and washed with ethyl acetate (100 mL). The filtrate was concentrated to give the crude product as a slightly yellow oil (902 mg). The crude product was purified by column chromatography on silica gel (0 to 10% methanol in chloroform). This gave the desired product D as a pale wax (492 mg, 0.58 mmol, 60%).

### Synthesis of IV-12:

To a solution of D (492 mg, 0.58 mmol) in DCM (5 mL) and DMF (8 mg) was added oxalyl chloride (3.2 mmol, 406 mg) at RT under Ar. This mixture was stirred at RT overnight and concentrated. The residue was taken up in DCM (5 mL) and concentrated again to remove any oxalyl chloride. The residual oil (viscous yellow oil) was dissolved in 10 mL of DCM was added via syringe to a solution of E (1.9 mmol, 356 mg) and triethylamine (750 uL) and DMAP (5 mg) in DCM (10 mL) at −15 C in 5 min. After addition, the mixture was allowed to rise to RT slowly and stirred overnight. After purification by column chromatography (0 to 5% methanol in chloroform), the desired compound IV-12 was obtained as a colorless oil (100 mg). ¹HNMR (400 MHz, CDCl3) δ: 4.08 (t-like, 7.1 Hz, 1H), 3.97 (d, 5.8 Hz, 4H), 3.54-3.44 (m, 4H), 3.23-3.17 (m, 2H), 2.44-2.33 (m, 8H), 2.30 (t, 7.5 Hz, 4H), 1.71-1.55 (m, 12H), 1.52-1.36 (m, 6H), 1.36-1.08 (70H), 0.92-0.86 (m, 15H).

### Example 3: Cleavage of the Mouse Albumin by Delivery of Nucleases Via LNP

To test the ability of targeted zinc finger nucleases to cleave a site in the mouse genome in vivo, mRNAs encoding albumin specific nucleases were incorporated into LNPs as described above using the cationic lipid I-6. These were then injected intravenously through the tail vein in C57BL6 mice as described above at a range of doses from 1 mg/kg to 30 mg/kg.

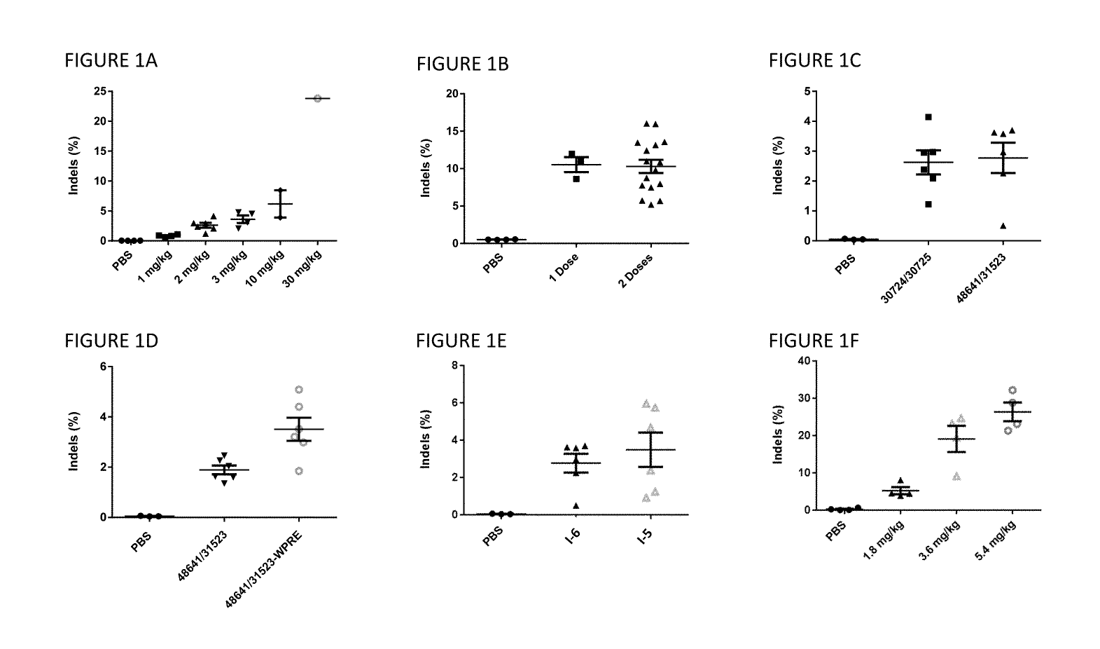

The data showed a dose response where use of higher amounts of ZFNs resulted in increased nuclease activity in the liver seven days after dosing (FIG. 1A, using 30724 and 30725 ZFN mRNA where the mRNAs comprised the dual HBB 3′ UTR, an ARCA cap and also 25% tU and 25% mC nucleoside substitution). Experiments were also done using a constant dose of 3 mg/kg comparing either a single dose or an initial dosing followed by a second dose 4, 7, 14, or 21 days later (see FIG. 1B). In these initial experiments, there was not any increase in nuclease activity detected due to the second dosing. LNPs comprising the 30724/30725 ZFN pair were also compared to a second albumin-specific pair 48641/31523 in the same LNP formulation comprising the I-6 cationic lipid, dosed at 2 mg/kg, and the results showed an equivalent level of nuclease activity in the liver for both LNP types (FIG. 1C).

Experiments were also performed to observe the nuclease activity that occurred when the individual ZFN mRNAs were supplied together as a single mRNA comprising a 2A-self cleaving peptide between them (FIG. 1D). The mRNA constructs either had the dual HBB 3′ UTR or the WPRE 3′ UTR. The animals were dosed at 2 mg/kg and the animals that had been dosed with the LNPs comprising the mRNAs including the WPRE 3′ UTR had higher observable nuclease activity in their livers. Additionally, LNPs comprising the 48641/31523 ZFN mRNAs where the LNPs comprising either the I-5 or I-6 cationic lipid were also compared for in vivo nuclease activity. In these experiments, animals were dosed with 2 mg/kg of the LNPs and the harvested livers showed very similar amounts of nuclease activity (FIG. 1E). Finally, the 48641/31523 ZFN mRNAs were incorporated into the I-5 LNPs and tested for activity using increasing doses of LNPs, from 1.8 mg/kg to 5.4 mg/kg (FIG. 1F), where increasing nuclease activity was found with increasing LNP dose.

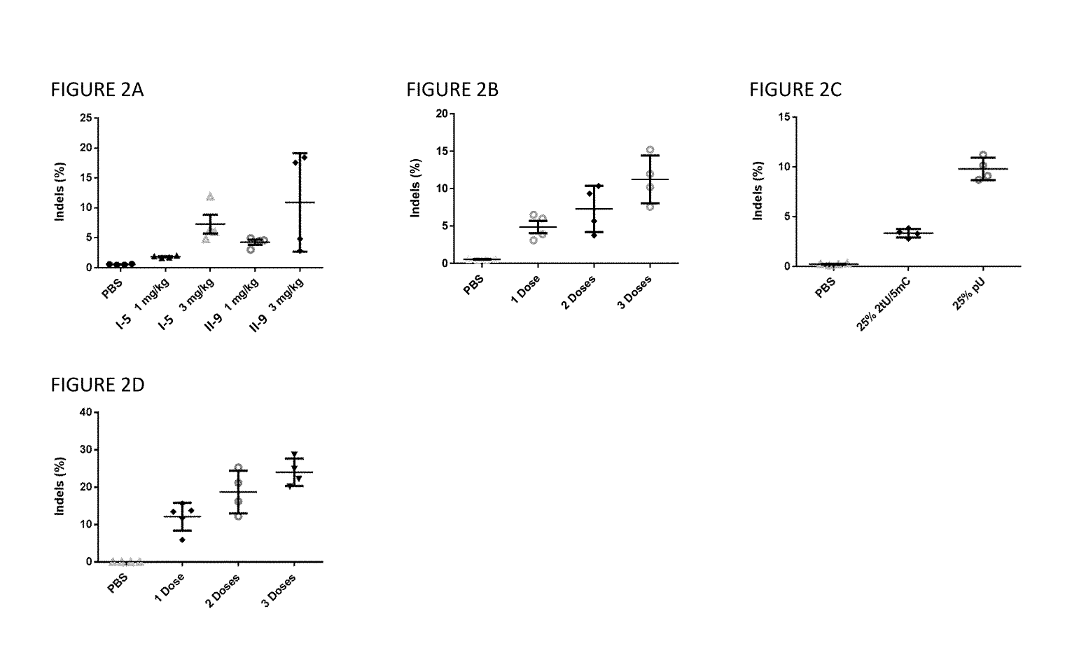

Experiments were done to compare the I-5 versus II-9 LNP formulations at two different doses (1 mg/kg or 3 mg/kg) using the 48641/31523 ZFN mRNA pair, where the mRNAs comprised the WPRE 3′ UTR and were ARCA-capped, and also comprised 25% 2tU & 25% 5mC nucleoside substitution. In these experiments (FIG. 2A), the increased amount of LNP dosed resulted in increased nuclease activity for both formulations. To further investigate the activity of multiple dosing, LNPs comprising either the I-5 or II-9 formulations and the mRNAs encoding the 48641/31523 ZFN pair were injected at 2 mg/kg at 28 day intervals (FIG. 2B).

The data indicated an increase in nuclease activity with the increasing doses. Experiments were also performed to investigate the use of other nucleoside compositions in the ZFN-encoding mRNAs incorporated into the LNPs. FIG. 2C shows the results comparing the pair made from mRNA comprising 25% 2tU/25% 5mC to mRNA comprising 25% pseudo-uridine (pU), dosed at 2 mg/kg using the II-9 cationic lipid LNP. In addition, the mRNAs comprising the 25% pseudo-uridine described above were tested for repeat dosing where each dose was given at 2 mg/kg, also in the II-9 LNP formulation, and the repeat doses were given at fourteen day intervals for up to a total of three doses. The data demonstrated that increased evidence of cutting was detectable upon the repeated dosing, reaching approximately 25% indels found in the target albumin genes after the third dose.

### Example 4: Role of Purification and Cap Structure in LNP Performance

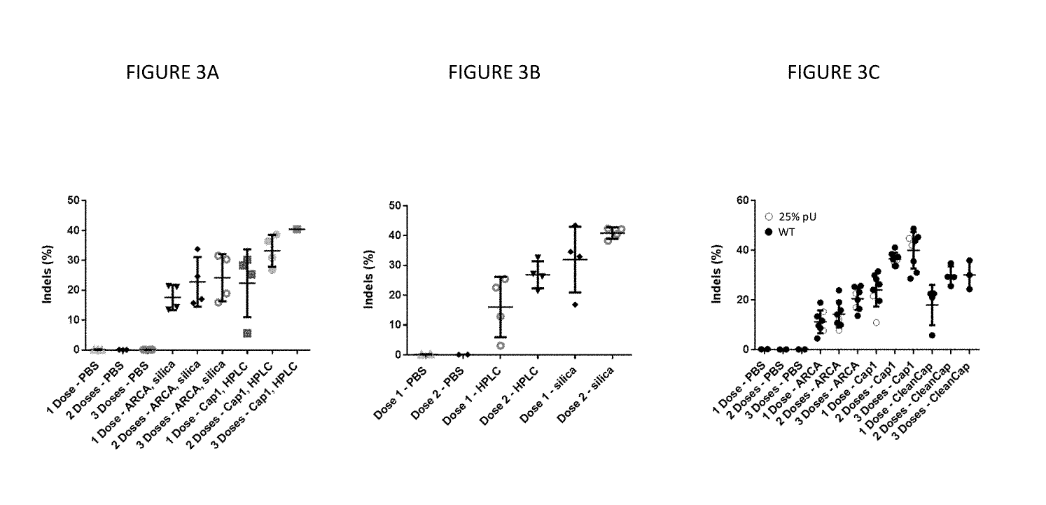

We next investigated the role that the method used for purification of the mRNAs played in the performance of the LNPs as well as the modifying what cap was incorporated into the mRNAs. Repeat dosing LNPs comprising mRNAs encoding ZFNs 48641/31523 (WPRE 3′ UTR, 25% pU) formulated in the II-9 cationic lipid formulation was done, where each dose was at 3.5 mg/kg and the dosing was done at 14-day intervals. In these experiments, animals were pretreated with 5 mg/kg dexamethasone 30 minutes prior to LNP dosing. In addition, we varied the cap structure between an ARCA cap, Cap1 or Cleancap. The data (FIG. 3) shows that the animals dosed with a total of three doses of mRNAs purified by HPLC and comprising the Cap1 cap worked better than those purified by silica chromatography comprising an ARCA cap (FIG. 3A). Repeat dosing was also performed at 14-day intervals using the 48641/31523 (WPRE 3′ UTR, 25% pU, Cap1) and dosed at 2 mg/kg where the animals were also pretreated with dexamethasone where the mRNAs were purified either via HPLC or silica chromatography.

The results (FIG. 3B) showed the silica purification regime gave the best nuclease activity in this experiment. Finally, the experiments were done with the silica purified mRNAs comprising either wild type mRNA base composition (“WT”) or 25% pU. These experiments were also done with three different cap types—ARCA, Cap1 or CleanCap where dosing was at 2 mg/kg of II-9 LNP formulation at 14-day intervals. In these studies (FIG. 3C), nucleases delivered with the Cap1 mRNAs exhibited the highest activity.

### Example 5: Use of Immunosuppression

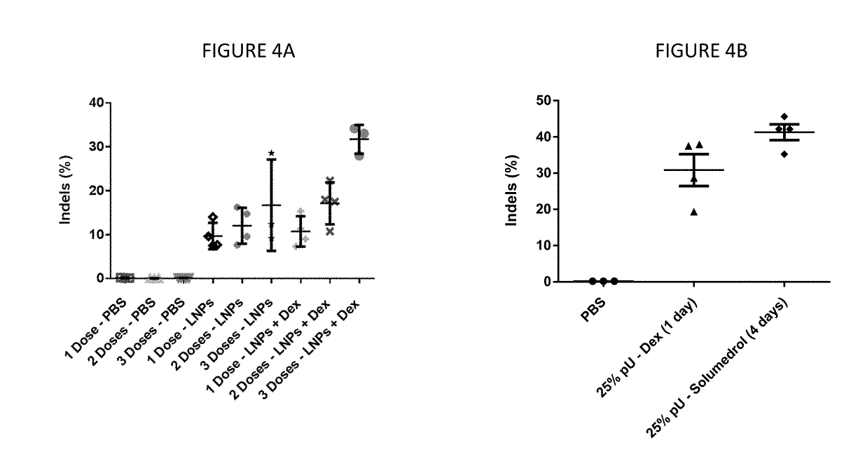

The work was continued, and explored the use of steroid treatment. Animals were subjected to repeat dosing at 14 day intervals of the 48641/31523 mRNA pair (WPRE 3′ UTR, 25% pU, ARCA capped and silica purified) at 2 mg/kg. The animals were either pre-treated with dexamethasone or not. The results (FIG. 4A) demonstrate that pretreatment with dexamethasone allows for greater detectable nuclease activity. Steroid treatment was further studied by comparing the results of pretreatment with dexamethasone with pretreatment and them 3 days treatment post-infection with Solumedrol®. We found that both treatments were effective with the Solumedrol® being slightly more efficacious (FIG. 4B).

### Example 6: In Vitro Transduction

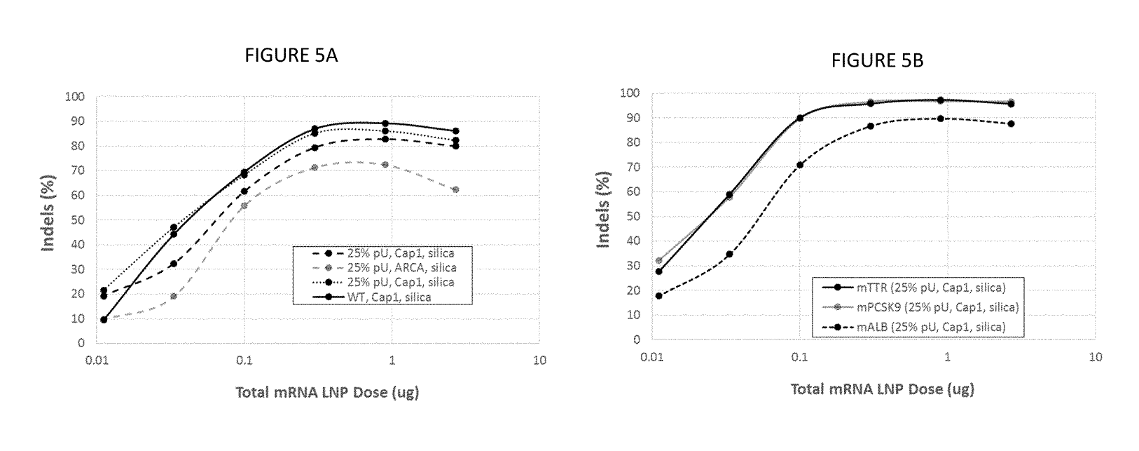

We also tested our LNP formulations in vitro to see if the nuclease activity could be detected in this setting. A range of LNP concentrations, in the LNP formulation comprising cationic lipid II-9, were tested in Hepa1-6 cells. FIG. 5A shows the experiments using the albumin specific 48641/31523 pair where the mRNAs comprises the WPRE 3′ UTR, and either wildtype RNA residues or 25% pU nucleoside substitution, and were either ARCA or Cap1 capped. The data demonstrates that all formulations were capable of introducing the nucleases into the cells and achieving excellent activity. Further, in vitro studies were also done in the II-9 formulation where ZFN pairs specific for mouse albumin (48641/31523), PCSK9 (58780/61748) or TTR (59771/59790) were tested.

The results (FIG. 5B) demonstrate that all LNP formulations were effective at transducing the cells in vitro with the mRNAs encoding the ZFNs.

### Example 7: In Vivo Targeting Mouse TTR and PCSK9

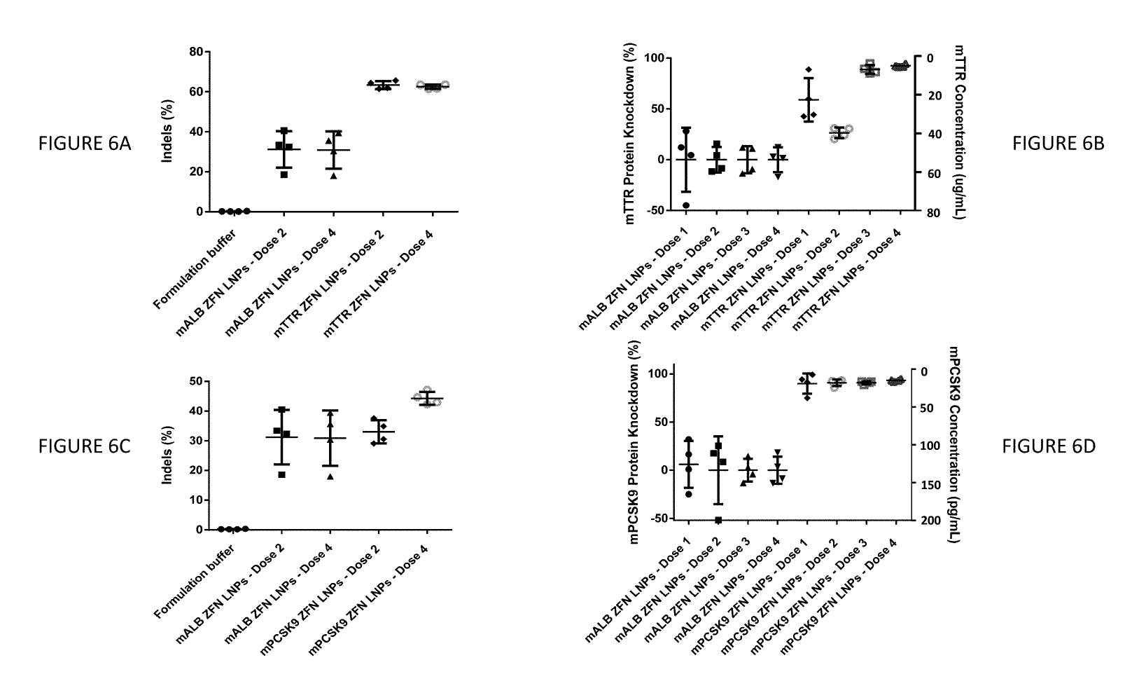

We next tested the ZFN pairs described in Example 6 in vivo using repeated dosing of 0.8 mg/kg of the ZFN encoding mRNA formulated into LNPs comprising the II-9 cationic lipid where the animals were pretreated with dexamethasone. A comparison of the TTR- and Albumin-specific LNPs was done where the 59771/59790 TTR and the 48641/31523 Albumin reagents were tested when the mRNAs comprised WPRE 3′ UTR and ARCA-caps, and the mRNA comprised 25% pU nucleoside substitution. The results are shown in FIG. 6A and demonstrated that both the mTTR and mALB-specific LNPs were active in vivo. Plasma from the mice treated with the TTR reagents (knock out mice) was collected and analyzed for TTR expression via ELISA as discussed in Example 2. The results (FIG. 6B) demonstrate a reduction in TTR protein detected by the ELISA in comparison with the mice treated with either buffer or with LNPs comprising Albumin targeting ZFN.

The experiments were also carried out using LNPs formulated with mouse PCSK9-specific ZFN-encoding mRNAs (58780/61748) in the II-9 cationic lipid formulation. As described above, the mRNAs comprised WPRE 3′ UTR and ARCA-caps, and the mRNA sequence comprised 25% pU nucleoside substitution. The animals were pretreated with dexamethasone and then subjected to repeat dosing with the LNPs and the results (FIG. 6C) demonstrated that the mALB and the mPCSK9-specific LNPs were active in vivo. Similar to the experiments with the TTR-specific ZFN, plasma was collected from the mice receiving the PCSK9-targeted LNPs and an ELISA was performed to evaluate the concentration of PCSK9 in the plasma following treatment. The results (FIG. 6D) showed that the amount of PCSK9 in the plasma was reduced as compared to the mice treated with the Albumin specific LNPs, indicating the nucleases were effective at cleaving their target in vivo.

### Example 8: Use of LNP Delivery of Targeted Nucleases In Vivo for Transgene Integration

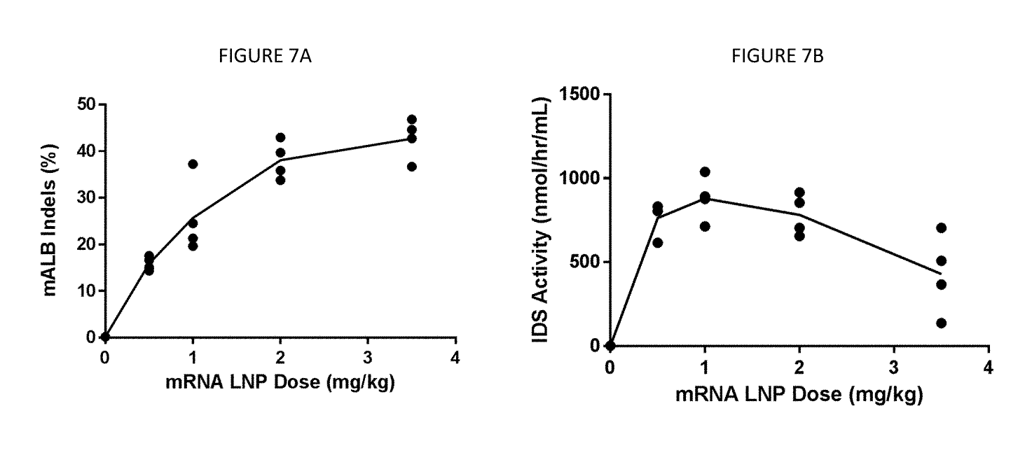

Use ZFNs delivered via LNPs in vivo in combination with a transgene was also explored. Mouse albumin-specific ZFN mRNAs (48641/31523) were delivered via LNP comprising the II-9 cationic lipid formulation where the mRNAs comprised a WPRE 3′ UTR and a Cap1, and the composition of the RNA sequence comprised 25% pU. The mice were treated intravenously with a range of doses (1-4 mg/kg) of the LNP along with 1.5e12 vector genomes of an AAV2/8 composition comprising a human IDS transgene donor with homology arms flanking the ZFN cut site and a splice acceptor just upstream of the transgene coding region. Animals were pre-treated with dexamethasone. The results (FIG. 7A) demonstrated nuclease activity up to 50% at the albumin locus, and IDS activity in the plasma (FIG. 7B) using the assay described in Example 2.

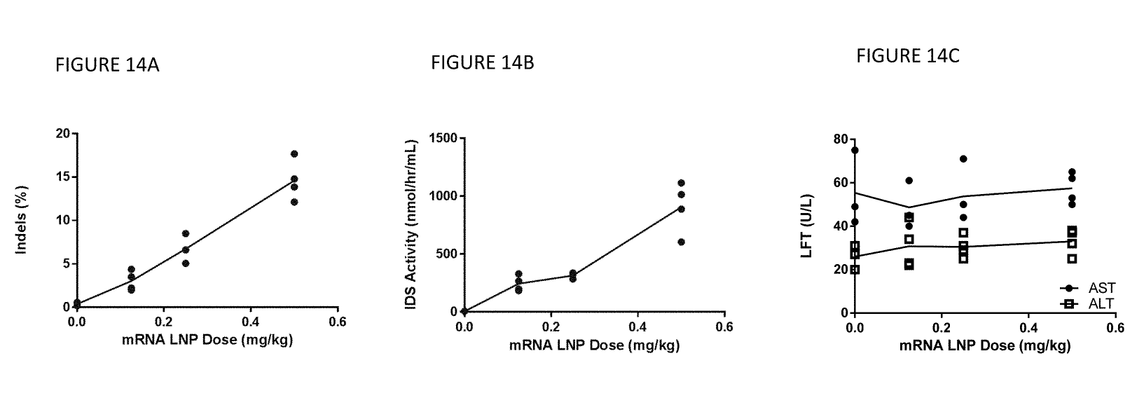

A study was also done using lower doses (up to 0.5 mg/kg) of the 48641/31523 LNP formulated comprising the II-9 cationic lipid formulation where the mRNAs comprised a WPRE 3′ UTR and a Cap1, silica purified, and the composition of the RNA sequence comprised 25% pU. As shown in FIG. 14, at these lower doses, approximately 15% liver indels were observed and approximately 800 nmol/hr/mL IDS activity was detected at the 0.5 mg/kg dose. Furthermore, analysis of liver enzymes (FIG. 14C) indicated that the doses were well tolerated in the subjects.

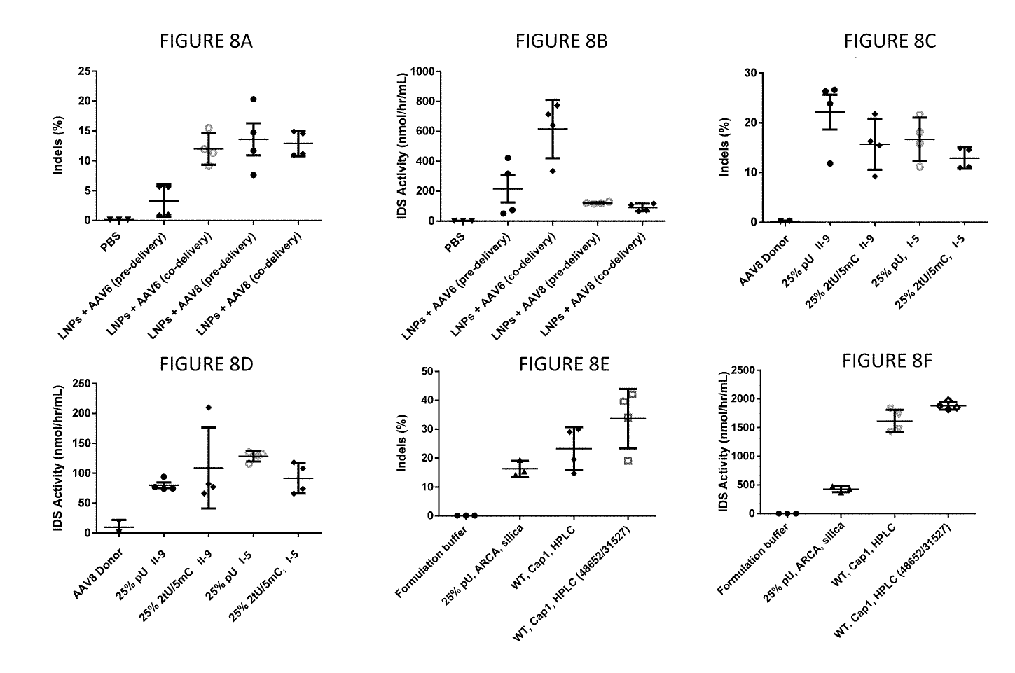

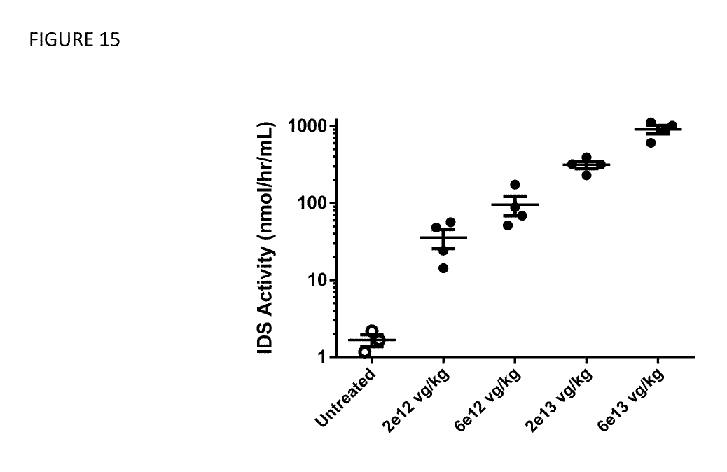

Another study to characterize more about the mRNA compositions was done using the mRNAs encoding the Albumin-specific ZFNs was done. 48641 and 31523 ZFN mRNA (WPRE 3′ UTR; 25% 2tU & 25% 5mC nucleoside substitution and ARCA-capped; silica-purified) were formulated into the LNP formulation comprising cationic lipid I-5 and injected into mice at 2 mg/kg either 1 day after (pre-delivery) or at the same time (co-delivery) as 1.5e12 vector genomes (vg) AAV2/6 or AAV2/8 encoding the human IDS transgene donor described above. Animals were pre-treated with dexamethasone. Livers were harvested for indel analysis, and the results (FIG. 8A) showed nuclease (indel) activity in the treated mice. These mice were then analyzed for plasma IDS activity (FIG. 8B). Finally, a range of donor AAV dosages was tested. In these experiments, the 48641/31523 LNP (formulated comprising the II-9 cationic lipid formulation where the mRNAs comprised a WPRE 3′ UTR and a Cap1, silica purified, and the composition of the RNA sequence comprised 25% pU) was given at a single 0.5 mg/kg dose, and the donor AAV2/8 was given over a range of 2e12 vg/kg to 6e13 vg/kg. IDS transgene activity in the plasma was then detected as described. The results (FIG. 15) demonstrated that the amount of IDS activity measured in the plasma displayed a dose-dependent response.

The data showed that the IDS activity was higher in the samples where the transgene was delivered by the AAV2/6.

A study was done using the Albumin-specific ZFN encoding mRNAs where 48641 and 31523 ZFN mRNA (WPRE 3′ UTR; 25% 2tU & 25% 5mC or 25% pU nucleoside substitution; ARCA-capped; silica-purified) were used. The mRNAs were mixed together and formulated in LNP formulation I-5 or II-9 and injected into mice at 2 mg/kg at the same time as 1.5e12 vector genomes (vg) AAV2/8 encoding the human IDS transgene donor above. Animals were pre-treated with dexamethasone. Livers were harvested for indel analysis.

The results (FIG. 8C) demonstrated that all formulations were active and that the II-9 LNP formulation comprising the mRNA with the 25% pU nucleoside composition was the most active. The plasma from those mice was also collected and analyzed for IDS activity (FIG. 8D). The results demonstrated that all samples comprising the LNPs and AAV donor were active.

Finally, a study was done using LNPs comprising either the 48641/31523 or the 48652/31527 ZFN encoding mRNA pairs (the mRNAs comprised WPRE 3′ UTR; unmodified residues or 25% pU nucleoside substitution; ARCA-capped or Cap1; silica- or HPLC-purified) formulated into LNP formulation II-9 and injected into mice at 2 mg/kg at the same time as 1.5e12 vector genomes (vg) AAV2/8 encoding the human IDS transgene donor. Animals were pre-treated with dexamethasone. Livers were harvested for indel analysis.

The nuclease activity results are shown in FIG. 8E and show that all compositions comprising the LNPs and donor AAV were active. In this experiment, the data sets labeled “25% pU, ARCA, silica” and “WT, Cap1, HPLC” are the results of experiments done with the Albumin-specific 48641/31523 comprising LNPs. Plasma from the treated mice was collected and analyzed for IDS activity (FIG. 8F). The results demonstrated that the formulations comprising wild type nucleosides and a Cap1 type of cap, purified by HPLC gave the highest activity.

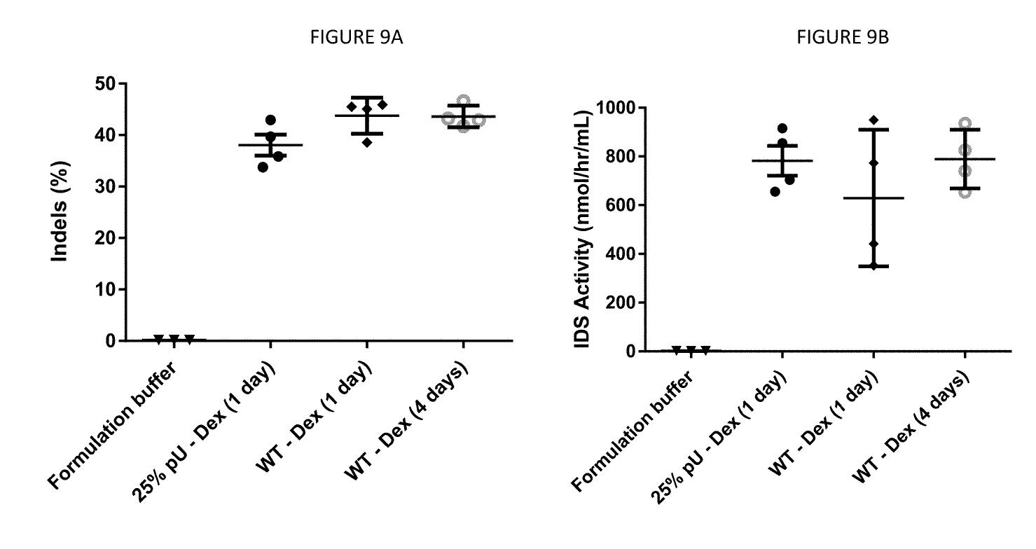

Additional experiments were done using the 48641/31523 pair to further characterize the effect of immunosuppression on the transgene integration. In these experiments, 48641 and 31523 ZFN mRNA (WPRE 3′ UTR; unmodified residues or 25% pU nucleoside substitution; Cap1; silica-purified) were mixed together and formulated into LNP formulation II-9 and injected into mice at 2 mg/kg at the same time as 1.5e12 vector genomes (vg) AAV8 encoding the human IDS transgene donor. Animals were either pre-treated with 5 mg/kg dexamethasone just prior to LNP dosing or just prior to and for an additional 3 days after LNP dosing at 5 mg/kg each time. Livers were harvested for indel analysis. The results (FIG. 9A) demonstrated nuclease activity in all samples comprising the LNP and AAV donor. Plasma was harvested from these mice and analyzes for IDS activity (FIG. 9B). All treated mice had IDS activity in their plasma.

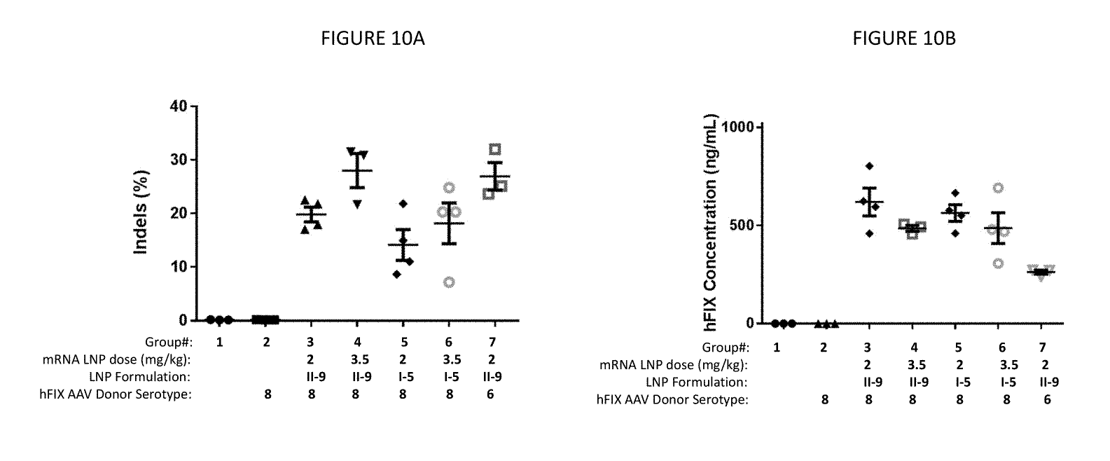

Experiments were also performed using a human Factor IX (hFIX) encoding donor in combination with the Albumin-specific ZFN. In these experiments, 48641 and 31523 ZFN mRNA (WPRE 3′ UTR; 25% pU nucleoside substitution; ARCA-capped; silica-purified) were mixed together and formulated into LNP formulation II-9 or I-5 and injected into mice at 2 or 3.5 mg/kg. At the same time, 1.5e12 vector genomes (vg) AAV8 comprising a human FIX transgene donor with homology arms flanking the ZFN cut site and a splice acceptor just upstream of the transgene coding region were injected. Animals were pre-treated with dexamethasone just prior to LNP dosing. Livers were harvested for indel analysis and the results are shown in FIG. 10A. The study showed that all mice receiving the LNP and AAV donors had nuclease activity. The plasma from the treated mice was analyzed for the presence of hFIX as described in Example 2, and the results are shown in FIG. 10B. All mice treated with the LNPs and AAV donors had detectable levels of hFIX in the plasma, including therapeutic levels of hFIX.

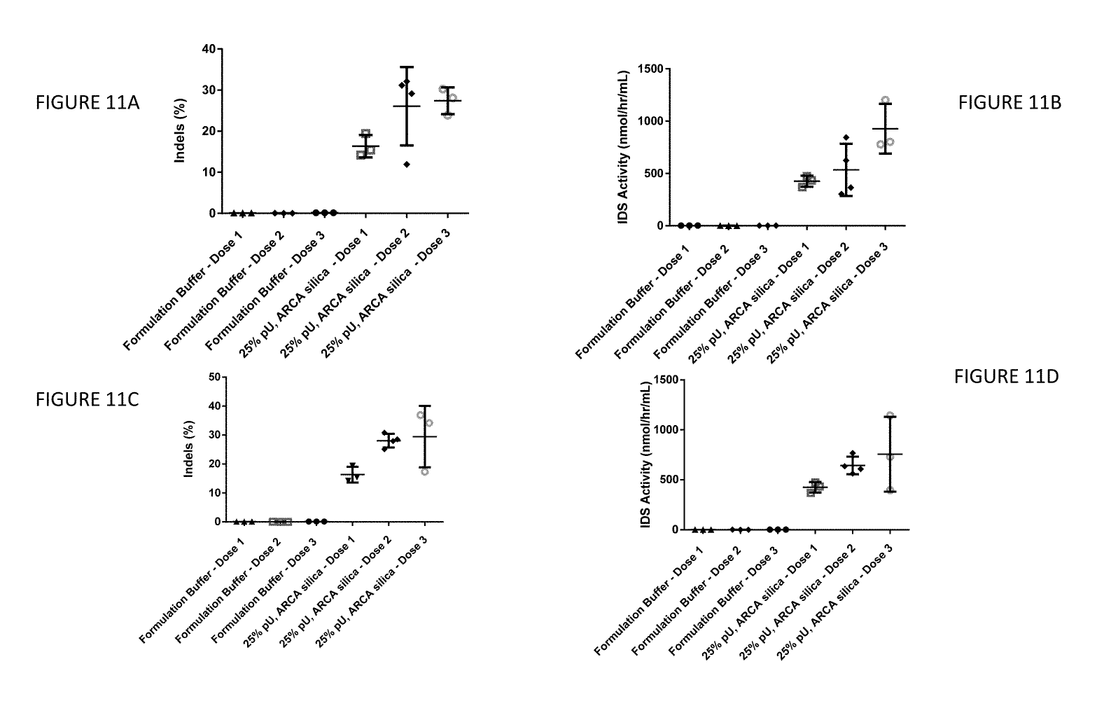

Repeat dosing was also performed where mice received the 48641/31523 ZFN pair via LNP delivery of mRNAs encoding the ZFNs formulated using the II-9 cationic lipid. The mRNAs comprised a WPRE 3′ UTR and an ARCA cap, and had a 25% pU nucleoside composition. The LNPs were injected at 2 mg/kg. The first dosing also included co-delivery of 1.5e12 vector genomes (vg) AAV8 encoding the human IDS transgene donor described above and animals were pre-treated with dexamethasone prior to each LNP dosing. Thus, the initial dose included the LNPs comprising the ZFN pair, and the AAV donor. Subsequent doses, done at 14 day intervals for a total of three doses. Livers were harvested for indel analysis, and the results are shown in FIG. 11A. The data demonstrated that all mice that received the LNPs and the AAV donor were active, and that the nuclease activity increased with a subsequent dose. Plasma was collected and assayed for IDS activity (FIG. 11B) and the results demonstrated that IDS activity was present in all animals receiving the donor and the LNPs, and that activity increased with each subsequence dose. Another set of studies was done shortening the dosing schedule to every 7 days. In these experiments, the same LNPs were used (48641 and 31523 ZFN mRNA, WPRE 3′ UTR; 25% pU nucleoside substitution; ARCA-capped; silica-purified), formulated comprising the II-9 cationic lipid where the mice were treated at a dose of 2 mg/kg LNP and 1.5e12 vector genomes (vg) AAV8 comprising the IDS donor at the first dose. The results are shown in FIG. 11C, and demonstrated that all mice that received the LNPs and AAV donor showed nuclease activity in the liver. Plasma was also harvested and IDS activity assayed as before (FIG. 11D). The results demonstrated that IDS activity was present in all samples receiving the LNP/AAV-donor treatment, and that activity increased over the dosing period.

Repeat dosing was also performed where mice received the 48641/31523 ZFN pair via LNP delivery of mRNAs encoding the ZFNs formulated using the II-9 cationic lipid. The mRNAs comprised a WPRE 3′ UTR, Cap1, were HPLC-purified, and had unmodified residues. The LNPs were injected at 0.5 mg/kg. The first dosing also included co-delivery of 1.5e12 vector genomes (vg) AAV8 or AAV6 encoding the human IDS transgene donor described above and animals were pre-treated with dexamethasone prior to each LNP dosing. Thus, the initial dose included the LNPs comprising the ZFN pair, and the AAV donor. Subsequent doses, done at 7-day intervals for a total of three doses.

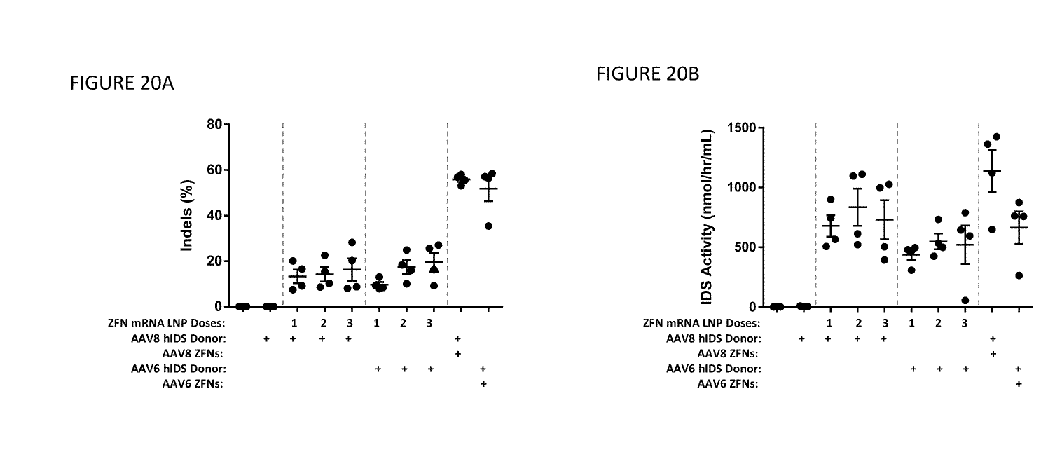

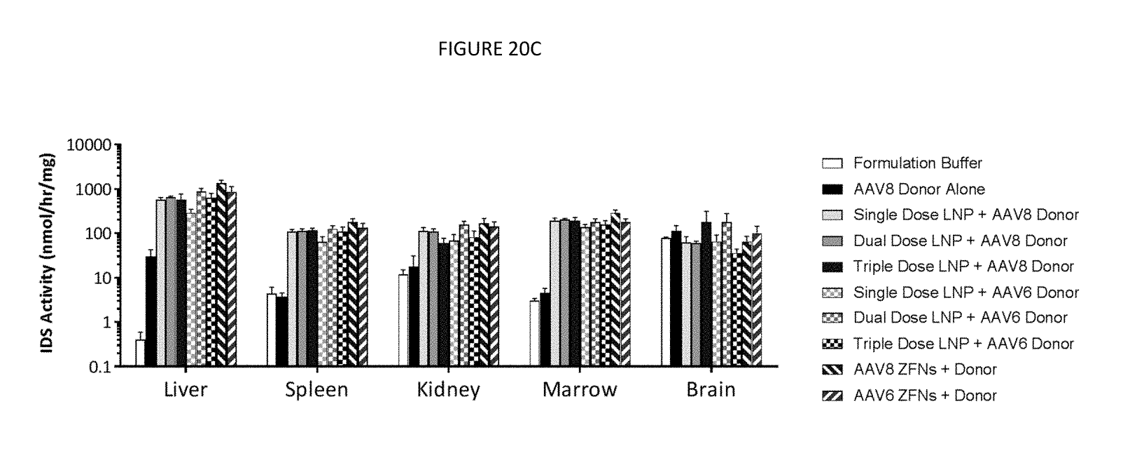

Livers were harvested for indel analysis, and the results are shown in FIG. 20A. The data demonstrated that all mice that received the LNPs and the AAV donor were active, and that the nuclease activity increased with a subsequent dose.

Plasma and tissues were collected and assayed for IDS activity (FIGS. 20B and 20C, respectively) and the results demonstrated that IDS activity was present in all animals receiving the donor and the LNPs, and that activity increased with each subsequent dose.

### Example 9: Intradermal Delivery of Nuclease

A study was performed where the LNPs comprising the nucleases were delivered to the mice intradermally. Briefly, LNPs comprising the 48641 and 31523 ZFN mRNAs were formulated using either the I-5 or II-9 cationic lipids. Mice were then shaved on their dorsal region and then injected intradermally with 50 uL of formulated mRNA LNPs diluted in PBS at a range of doses. Mice were sacrificed and nuclease activity in the skin immediately surrounding the injection site was harvested and analyzed as described above.

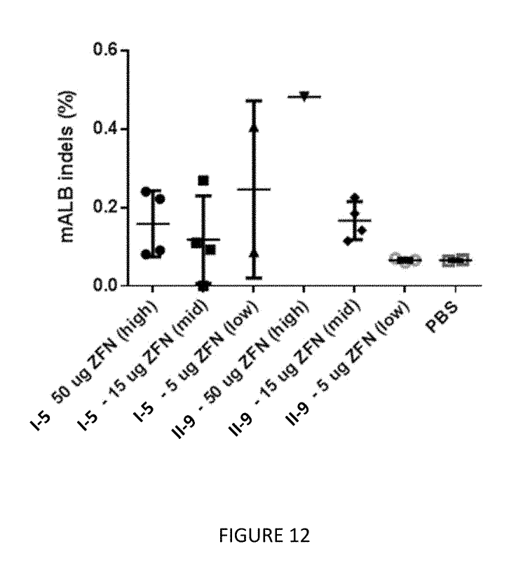

The results (FIG. 12) demonstrate that the mice that received the LNPs had detectable nuclease activity in the skin surrounding the injection site.

### Example 10: In Vivo Targeting Mouse Albumin

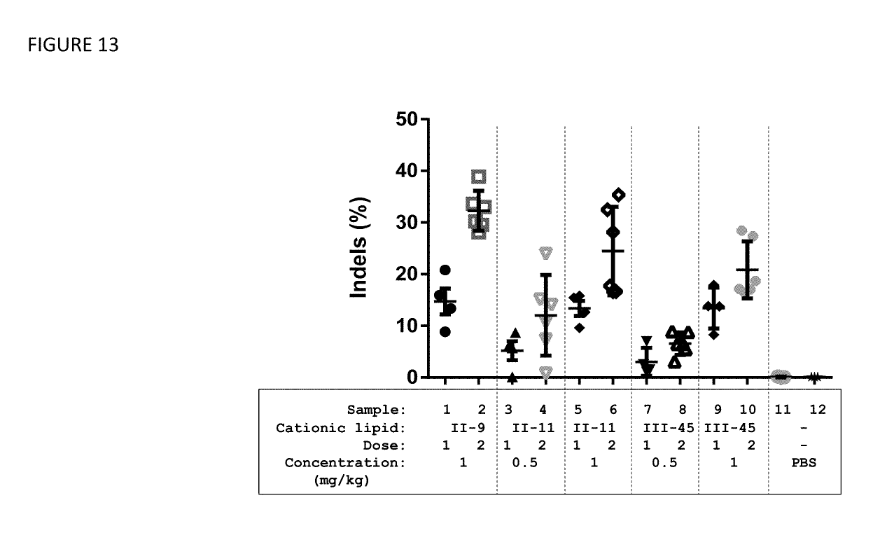

Mouse albumin was targeted using various different formulations, II-9, II-11 or III-45 cationic lipids (FIG. 13) and II-9, II-36, III-25, III-45, III-49, or IV-12 (FIG. 23). All were formulated at an intermediate amino lipid (N):mRNA (P) ratio. In FIG. 23, Formulation II-9 was also formulated at low and high N:P ratios as well as at an intermediate N:P ratio but a larger LNP of ˜100 nm. ZFNs 48641 and 31523 were used as described above where the mRNAs encoding the ZFN pairs comprised 25% pU substitutions, WPRE and Cap1, and were purified by silica. The mice were pretreated with dexamethasone (5 mg/kg) 30 minutes prior to dosing. Dose repeats were done 14 days apart, and the mice were sacrificed 7 days following the second dose. The data (FIGS. 13 and 23) showed that all LNP formulations were active.

Experiments were also performed using the LNP II-9 formulation containing ZFNs 48641 and 31523 where the mRNAs encoding the ZFN pairs comprised 25% pU substitutions, WPRE 3′ UTR, Cap1, and were purified by either silica membrane (0.5 mg/kg LNP dose) or HPLC-purification (2.0 mg/kg dose). The mice were pretreated with dexamethasone (5 mg/kg) 30 minutes prior to each LNP dosing. Dose repeats were done 14 days apart, and a cohort of mice were sacrificed 7 days following each dose.

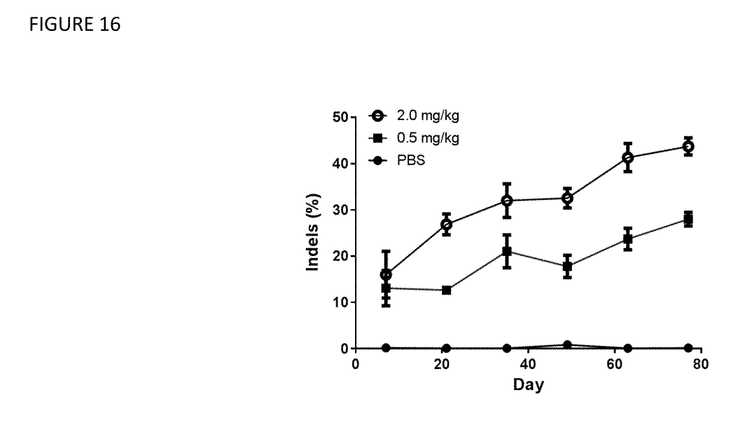

As shown in FIG. 16, an accumulation of genome modification (indels) following each subsequent LNP dose was observed.

Experiments were also performed using the LNP II-9 formulation comparing 25% pU substituted or unmodified mRNAs encoding ZFNs 48641 and 3152, where the mRNAs comprised WPRE 3′ UTR, Cap1, and silica-purified. The mice were pretreated with dexamethasone (5 mg/kg) 30 minutes prior to dosing. Mice were dose at 0.5 mg/kg LNP and livers were harvested for genome modification analysis 7 days later.

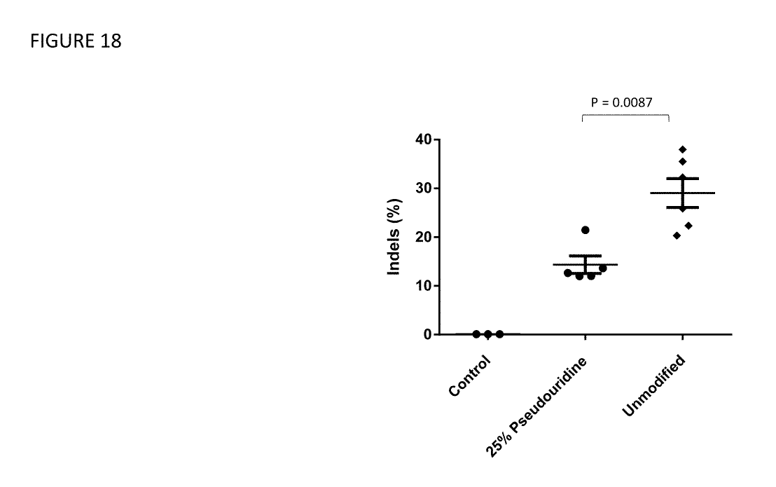

As shown in FIG. 18, unmodified ZFN mRNA yielded higher genome modification levels than the 25% pU substituted sample.

### Example 11: Biodistribution of Genome Modification

Mouse albumin was targeted using the LNP II-9 formulation containing ZFNs 48641 and 31523 where the mRNAs encoding the ZFN pairs comprised 25% pU substitution, WPRE 3′ UTR, Cap1, and silica membrane purification. The mice were pretreated with dexamethasone (5 mg/kg) 30 minutes prior to LNP dosing. Mice were dosed at 2.0 mg/kg LNP and mice were sacrificed 7 days later and livers, bone marrow, and spleens were harvested for genome modification analysis.

In addition, a cohort of mice was either sacrificed and unmanipulated prior to liver harvest (“unperfused”) or perfused transcardially with buffered saline prior to liver harvest to remove blood cells within the liver (“unsorted”). A fraction of the perfused liver was digested with collagenase to create a single cell suspension, then fluorescently immunostained with a kupffer cell-specific marker and an endothelial cell-specific marker. Stained cells were then FACS-sorted into endothelial cell marker positive, kupffer cell marker positive, or marker negative (hepatocyte) cell populations. Genomic DNA was then harvested from these sorted cells and analyzed for genome modification (indels).

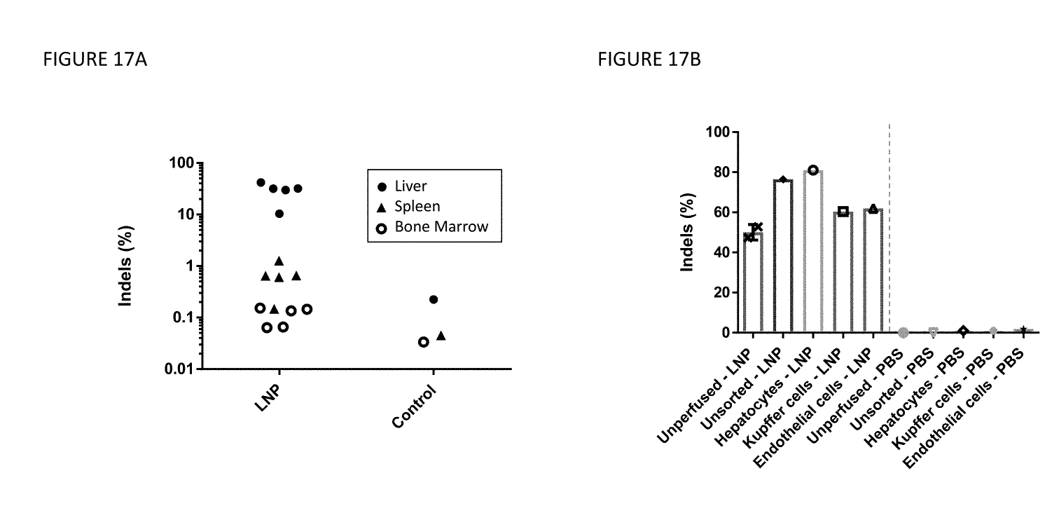

FIG. 17A shows genome modification (indels) in the various organs (liver, spleen and bone marrow). FIG. 17B shows genome modification in mice bulk liver tissue is substantially lower than in perfused mice, likely due to presence of untargeted nucleated cells within the blood. Additionally, hepatocytes are the cell population which are most highly targeted within the liver.

### Example 12: Fasting Animals Prior to LNP Dosing

Mouse albumin was targeted using formulation II-9 cationic lipid (LNP II-9 formulation) containing ZFNs 48641 and 31523 where the mRNAs encoding the ZFN pairs comprised 25% pU substitution, WPRE 3′ UTR, Cap1, and silica membrane purification. A cohort of animals were denied access to food overnight for approximately 16 hours prior to the beginning of the immunosuppression and LNP dosing. All mice were pretreated with dexamethasone (5 mg/kg) 30 minutes prior to LNP dosing. Mice were dosed at 0.5 mg/kg LNP and mice were sacrificed 7 days later and livers were harvested for genome modification analysis.

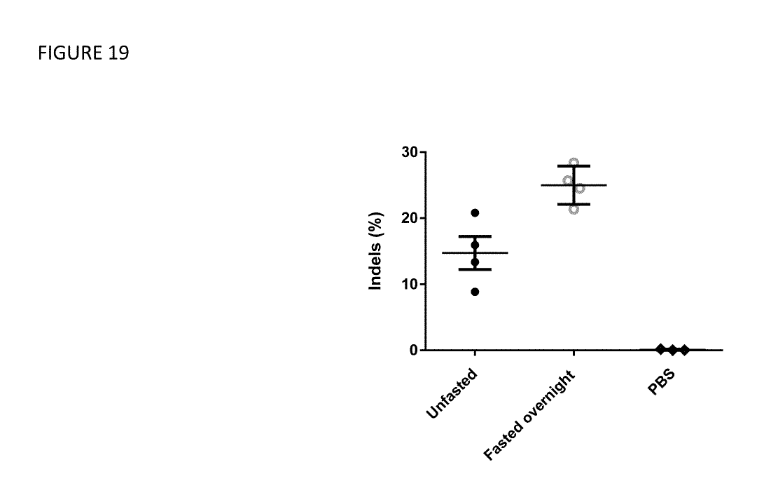

As shown in FIG. 19, genome modification (indels) in animals which were fasted overnight was higher than animals which had access to food ad libitum overnight prior to LNP dosing.

### Example 13: Extending the polyA Tail and Removing Uridines from Coding Region of mRNA Transcript

Mouse albumin was targeted using formulation II-9 cationic lipid containing ZFNs 48641 and 31523 where the mRNAs encoding the ZFN pairs comprised unmodified residues, WPRE 3′ UTR, Cap1, and silica membrane purification. All mice were pretreated with dexamethasone (5 mg/kg) 30 minutes prior to LNP dosing. Mice were dosed at 0.5 mg/kg LNP and mice were sacrificed 7 days later and livers were harvested for genome modification analysis. Mice which were repeatedly dosed were dosed at 14-day intervals.

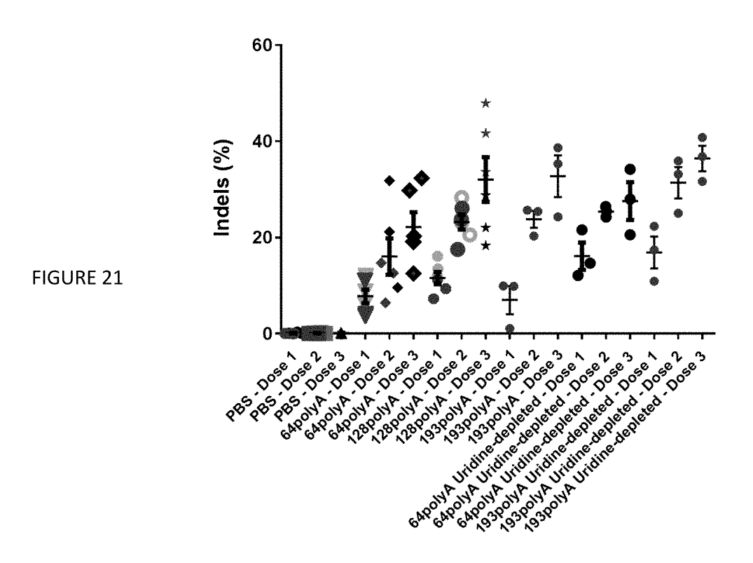

As shown in FIG. 21, the highest levels of genome modification (indels) in animals was obtained when longer polyA tails were present on the mRNAs, and when as many uridines as possible are removed (replaced) at the wobble positions in the coding region (˜50-60 uridines removed from ˜1250 bp coding region) of the mRNA transcript while retaining the same resulting amino acid sequence.

### Example 14: Optimized ZFN Constructs

Mouse TTR was targeted using formulation II-9 cationic lipid containing ZFNs 69121/69128 and 69052/69102 where the mRNAs encoding the ZFN pairs comprised unmodified residues, WPRE 3′ UTR, Cap1, 193polyA tail, uridine-depleted coding domain, and silica membrane purification. All mice were pretreated with dexamethasone (5 mg/kg) 30 minutes prior to LNP dosing. Mice were dosed at a range of LNP doses and mice were sacrificed 35 days following the initial LNP dose and livers were harvested for genome modification analysis.

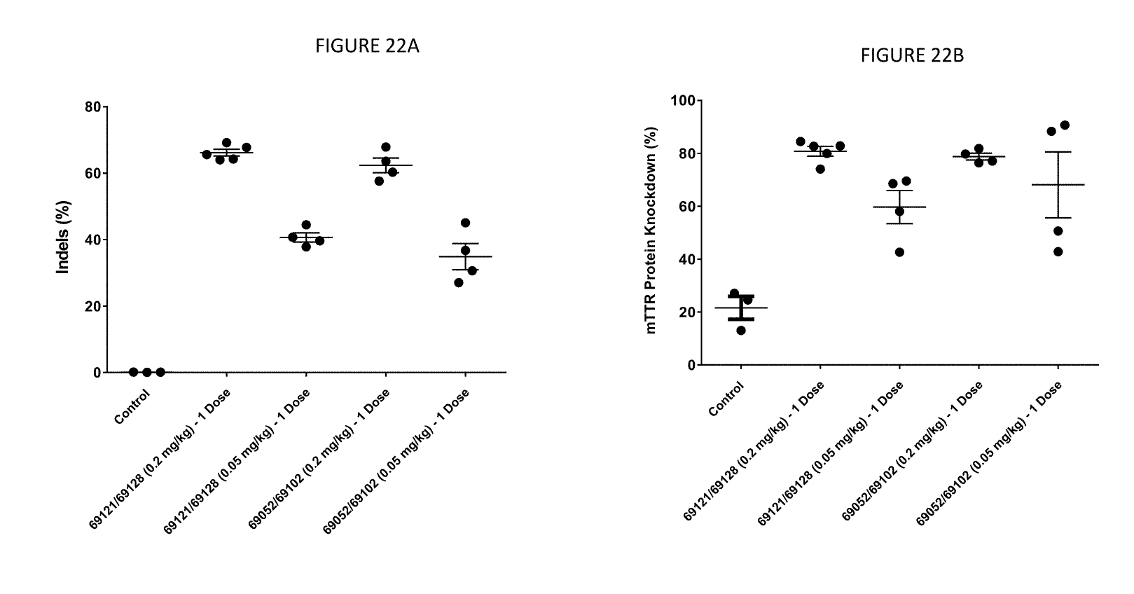

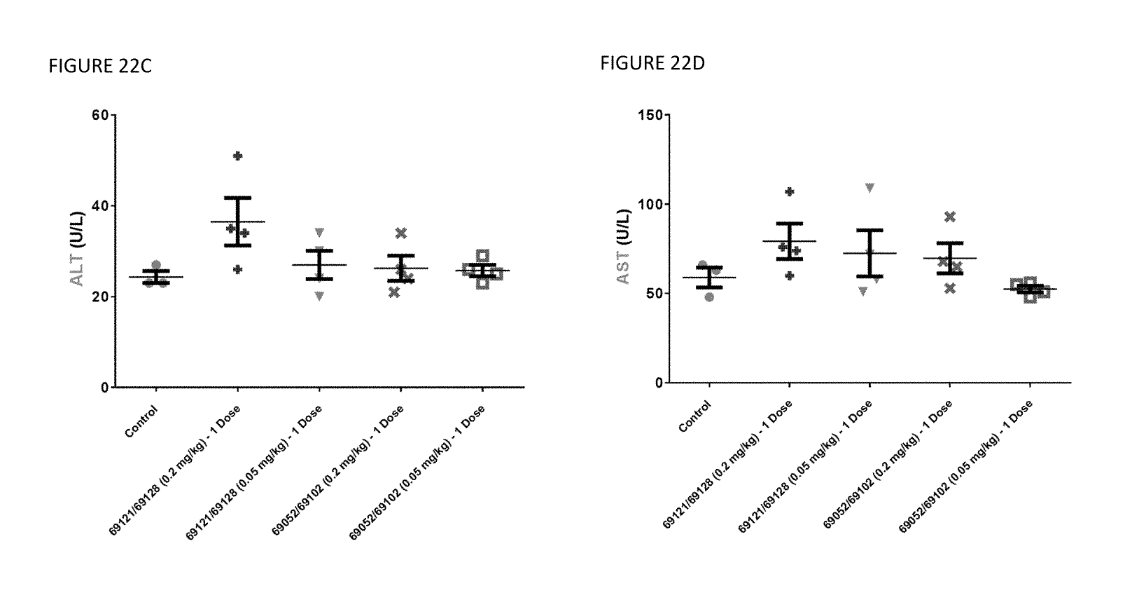

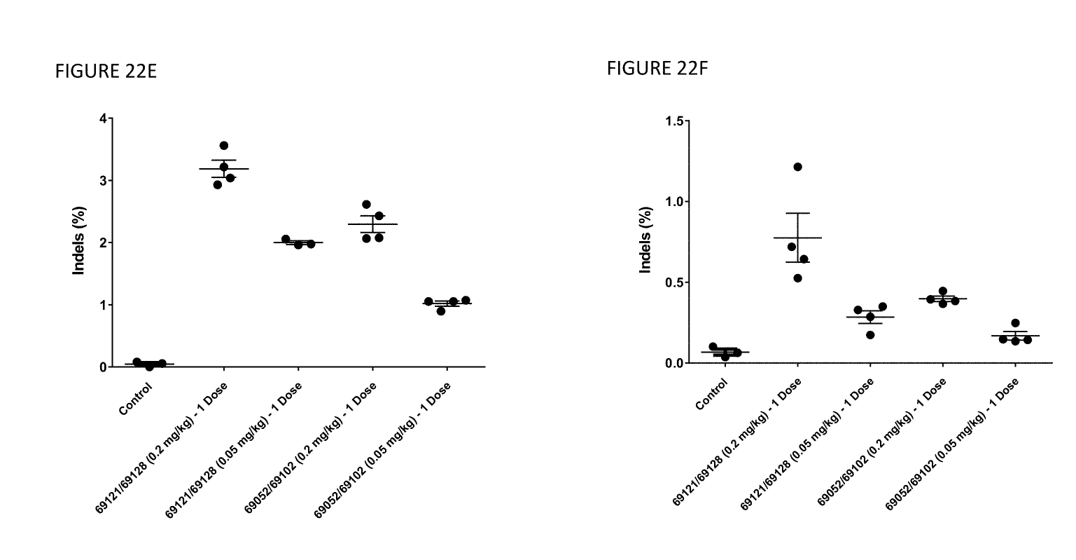

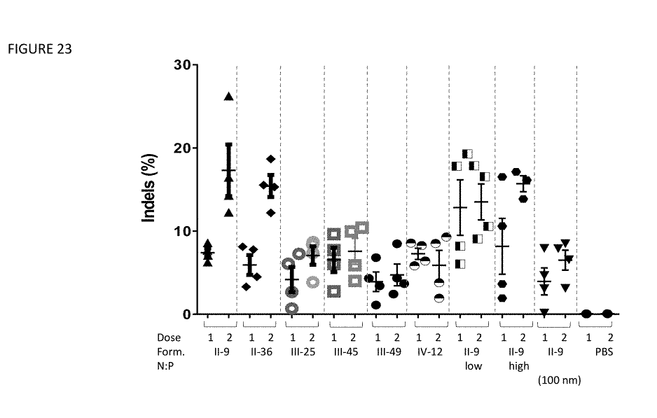

As shown in FIG. 22A, on-target genome modification (indels) was observed at the intended murine TTR locus in animals. FIG. 22B shows murine TTR ELISA assay in plasma collected from the mice described in FIG. 22A. FIGS. 22C and 22D shows results of liver function test (LFT) in serum collected from the mice described in FIG. 22A one-day post-dosing. “LFT” refers to liver function test; “ALT” refers to alanine transaminase; “AST” refers to aspartate transaminase. FIGS. 22E and 22F show minimal genome editing in off-target organs, spleen and kidney, respectively.

The results demonstrated dose-dependent on-target cleavage in vivo as well as dose dependent knockdown of mTTR protein expression. Additionally, treatment of the animals with the LNPs did not cause any notable changes in liver function.

These experiments have demonstrated that delivery of mRNAs encoding specific nucleases via LNP can result in targeted cleavage in the liver and skin of treated animals, and that inclusion of a donor transgene can result in the targeted integration and expression of the transgene, including from the livers, in vivo in treated animals. In particular, co-delivering the mRNA-LNP with AAV comprising either a promoterless human IDS or FIX transgene donor resulted in therapeutically-relevant levels of enzymatic activity (1950 nmol/hour/mL) and protein expression (1015 ng/mL), respectively, within the plasma (up to 7700-fold wild type levels, and 8-fold higher than in previous mouse studies for human IDS). In addition, repeat administration of the mRNA-LNP after a single AAV donor dose significantly increased levels of genome editing and transgene expression (approximately double after 2-3 doses). For gene knockout applications, ZFNs targeted to the TTR gene (a clinically-validated gene knockout/knockdown target for treatment of transthyretin-related amyloidosis) delivered as mRNA via electroporation, were capable of yielding >99% indels within murine liver cell lines in vitro. These ZFNs were then produced as mRNA, packaged into LNP, and injected intravenously into wildtype mice. After a single dose (0.2 mg/kg), 66% indels in liver tissue and 81% protein knockdown in plasma were observed with no significant increase in liver-associated transaminases within the serum.

In sum, LNP-mediated ZFN mRNA delivery drives highly efficient levels of in vivo genome editing, including for genome editing that provides therapeutic transgenes for treatment and/or prevention of subjects with genetic diseases.

What is claimed is:

1. A lipid nanoparticle (LNP) comprising one or more polynucleotides that encode one or more transgenes.

2. The LNP of claim 1, wherein the one or more transgenes encode one or more engineered nucleases, one or more engineered transcription factors, one or more transgenes encoding therapeutic proteins, or combinations thereof.

3. The LNP of claim 1, wherein the polynucleotides are randomly integrated into the genome, integrated in a targeted manner into the genome or expressed episomally in a cell.

4. The LNP of claim 2, wherein the nucleases and the transcription factors comprise a DNA-binding domain comprising a zinc finger protein, a TAL-effector domain or a single guide RNA, the nucleases further comprise a cleavage domain, and the transcription factors further comprise a transcriptional regulatory domain.

5. The LNP of claim 1, wherein the polynucleotides comprise DNA, RNA or both.

6. The LNP of claim 5, wherein the RNA is mRNA and the DNA is a plasmid, a minigene, or a linear DNA.

7. The LNP of claim 1, comprising a first polynucleotide encoding a nuclease and a second polynucleotide comprising a transgene.

8. The LNP of claim 7, wherein administration of the LNP to a cell, and expression of the nuclease therein, results in targeted integration of the transgene into the genome of a cell.

9. The LNP of claim 1, comprising cationic lipid molecules and optionally neutral lipids, charged lipids, steroids, steroid analogs, polymer conjugated lipids, or combinations thereof.

10. The LNP of claim 9, wherein the cationic lipid is selected from compounds having the following Formulas (I, II, III and IV): 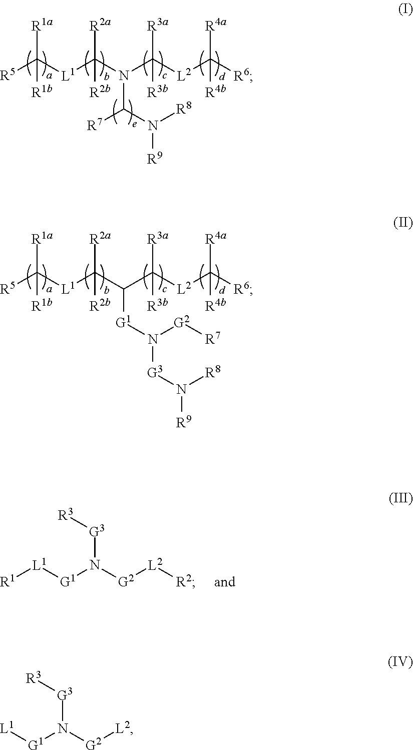
or a pharmaceutically acceptable salt, tautomer or stereoisomer thereof, wherein, for Formula (I):
L¹ and L² are each independently —O(C═O)—, (C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2,
for Formula (II):
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2,
for Formula (III):
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2,
and for Formula (IV):
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

11. The LNP of claim 10, comprising one of the following compounds I-5, I-6, II-9, II-11, II-36, III-25, III-45, III-49 or IV-12:

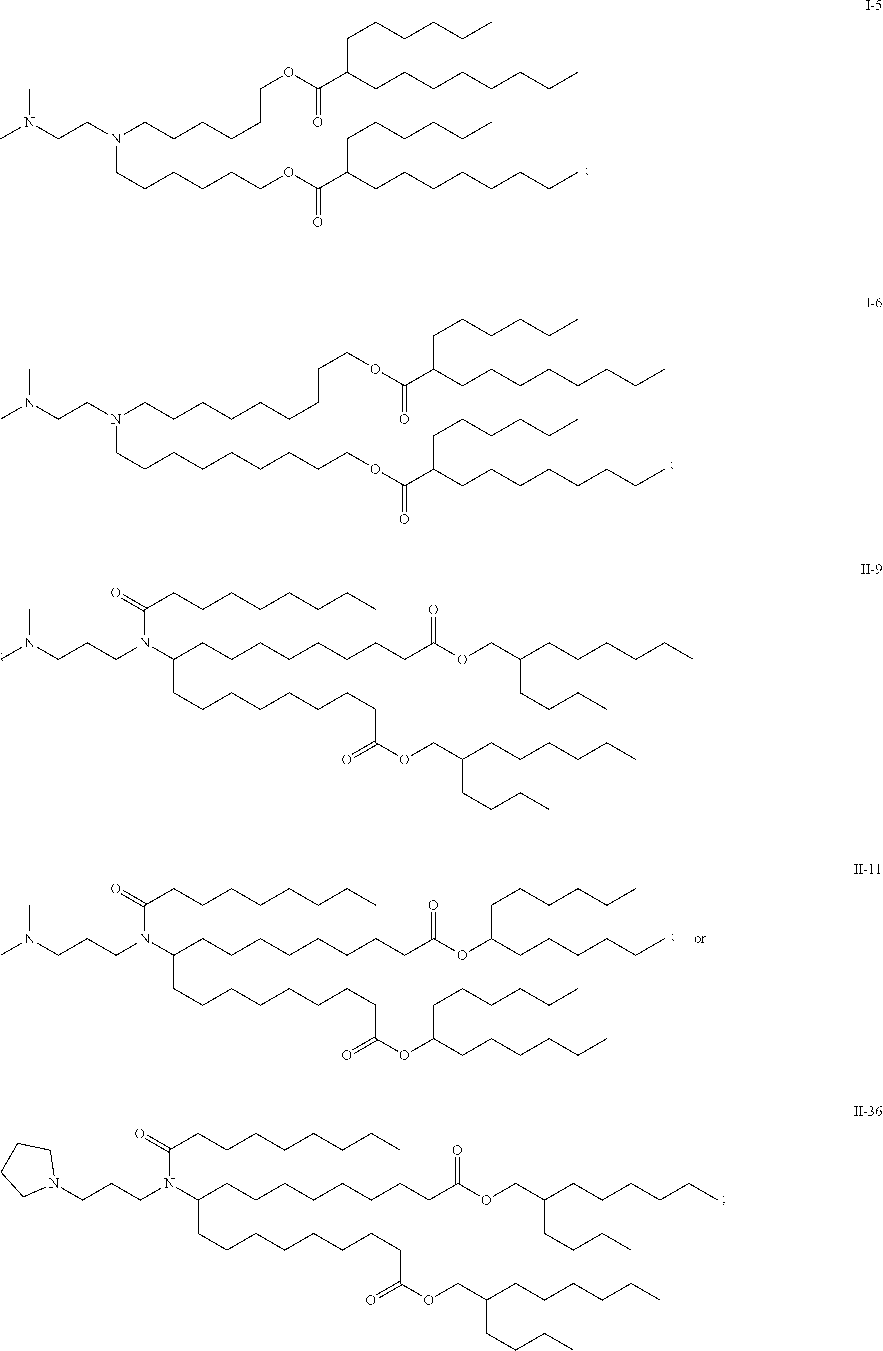

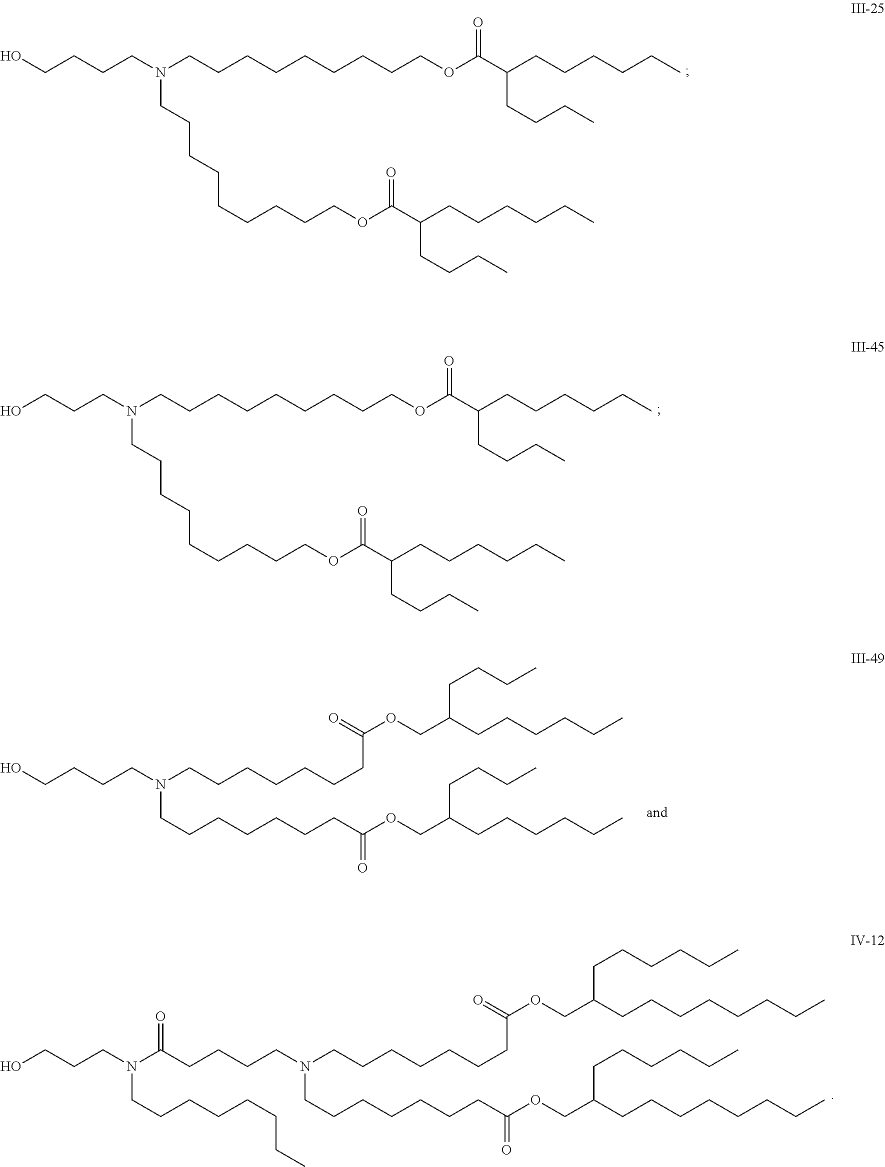 [SMILES: CCCCCCC(CCCC)C(=O)OCCCCCCCCCN(CCCCO)CCCCCCCCCOC(=O)C(CCCC)CCCCCC ; CCCCCCC(CCCC)C(=O)OCCCCCCCCCN(CCCO)CCCCCCCCCOC(=O)C(CCCC)CCCCCC ; CCCCCCC(CCCC)COC(=O)CCCCCCCN(CCCCO)CCCCCCCC(=O)OCC(CCCC)CCCCCC ; CCCCCCCCC(CCCCCC)COC(=O)CCCCCCCN(CCCCCCCC(=O)OCC(CCCCCC)CCCCCCCC)CCCCC(=O)N(CCCO)CCCCCCCC]

12. The LNP of claim 1, comprising a pegylated lipid having the structure of formula (V), 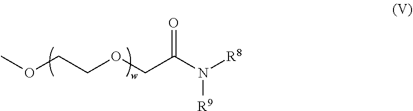
wherein R⁸ and R⁹ are each independently straight, saturated alkyl chains containing from 12 to 16 carbon atoms and w is 42 to 55.

13. A pharmaceutical composition comprising one or more LNPs according to claim 1.

14. The pharmaceutical composition of claim 13, further comprising a viral vector.

15. The pharmaceutical composition of claim 14, wherein the viral vector comprises a transgene donor.

16. A cell comprising one or more LNPs according to claim 1.

17. A cell descended from the cell of claim 16.

18. A cell of claim 16, wherein the cell is genetically modified, the genetic modification comprising an insertion, a deletion, or both.

19. A method of delivering one or more polynucleotides to a cell or a subject, the method comprising administering one or more LNPs according to claim 1 to the cell or the subject.

20. A method of cleaving a region of interest in a cell's genome, the method comprising delivering one or more LNPs according to claim 1, or a pharmaceutical composition comprising the one or more LNPs, to the cell, wherein at least one LNP comprises a polynucleotide encoding a nuclease that cleaves the genome.

21. The method of 20, wherein the region of interest is in a safe harbor gene.

22. The method of claim 20, wherein the safe harbor gene is an AAVS1 gene, an albumin gene, a Rosa gene, a CCR5 gene, a CXCR gene or an HPRT gene.

23. The method of claim 20, wherein the one or more LNPs comprise a donor comprising a transgene and the transgene is integrated into the genome of the cell following cleavage by the nuclease.

24. The method of claim 20, wherein the method further comprises delivering one or more viral vectors comprising at least one transgene to the cell, and the transgene is inserted into the genome of the cell following cleavage by the nuclease.

25. The method of treating a patient in need thereof, the method comprising administering one or more LNPs according to the method of claim 20 to the patient.

26. The method of treating a patient in need thereof, the method comprising administering one or more LNPs and viral vectors according to the method of claim 24.

27. The method of claim 23, wherein the method comprises sequentially and/or repeatedly administering the one or more LNPs comprising the nuclease and the one or more LNPs comprising the transgene.

28. The method of claim 24, wherein the method comprises sequentially and/or repeatedly administering the one or more LNPs comprising the nuclease and the one or more viral vectors.

29. The method of claim 19, wherein the method is performed in vitro, ex vivo, or in vivo.

30. The method of claim 29, wherein the LNPs are administered two or more times.

31. The method of claim 25, wherein the LNPs or pharmaceutical composition comprising the LNPs are administered to a patient and re-administered 7, 14, 21, 28, 30, 40, 50, 75, 100, and/or 200 or more days, or combinations thereof, after the initial administration.

32. The method of claim 26, wherein the LNPs or a pharmaceutical composition comprising the LNPs are administered to a patient and re-administered 7, 14, 21, 28, 30, 40, 50, 75, 100, and/or 200 or more days, or combinations thereof, after the initial administration.

33. A kit comprising one or more LNPs according to claim 1.


SEQUENCE LISTING (48 sequences)

SEQ ID NO: 1 - protein, 7 aa, Artificial Sequence
  Description of Artificial Sequence Synthetic peptide
TSGSLTR

SEQ ID NO: 2 - protein, 7 aa, Artificial Sequence
  Description of Artificial Sequence Synthetic peptide
RSDALST

SEQ ID NO: 3 - protein, 7 aa, Artificial Sequence
  Description of Artificial Sequence Synthetic peptide
QSATRTK

SEQ ID NO: 4 - protein, 7 aa, Artificial Sequence
  Description of Artificial Sequence Synthetic peptide
TSGHLSR

SEQ ID NO: 5 - protein, 7 aa, Artificial Sequence
  Description of Artificial Sequence Synthetic peptide
QSGNLAR

SEQ ID NO: 6 - protein, 7 aa, Artificial Sequence
  Description of Artificial Sequence Synthetic peptide
RSDHLSA

SEQ ID NO: 7 - protein, 7 aa, Artificial Sequence
  Description of Artificial Sequence Synthetic peptide
TKSNRTK

SEQ ID NO: 8 - protein, 7 aa, Artificial Sequence
  Description of Artificial Sequence Synthetic peptide
DRSNLSR

SEQ ID NO: 9 - protein, 7 aa, Artificial Sequence
  Description of Artificial Sequence Synthetic peptide
WRSSLRA

SEQ ID NO: 10 - protein, 7 aa, Artificial Sequence
  Description of Artificial Sequence Synthetic peptide
DSSDRKK

SEQ ID NO: 11 - protein, 7 aa, Artificial Sequence
  Description of Artificial Sequence Synthetic peptide
LRHHLTR

SEQ ID NO: 12 - protein, 7 aa, Artificial Sequence
  Description of Artificial Sequence Synthetic peptide
QAGQRRV

SEQ ID NO: 13 - protein, 7 aa, Artificial Sequence
  Description of Artificial Sequence Synthetic peptide
RSDNLSE

SEQ ID NO: 14 - protein, 7 aa, Artificial Sequence
  Description of Artificial Sequence Synthetic peptide
QSSNLAR

SEQ ID NO: 15 - protein, 7 aa, Artificial Sequence
  Description of Artificial Sequence Synthetic peptide
QSGHLSR

SEQ ID NO: 16 - protein, 7 aa, Artificial Sequence
  Description of Artificial Sequence Synthetic peptide
QSSDLSR

SEQ ID NO: 17 - protein, 7 aa, Artificial Sequence
  Description of Artificial Sequence Synthetic peptide
ASKTRKN

SEQ ID NO: 18 - protein, 7 aa, Artificial Sequence
  Description of Artificial Sequence Synthetic peptide
QSGHLAR

SEQ ID NO: 19 - protein, 7 aa, Artificial Sequence
  Description of Artificial Sequence Synthetic peptide
QLTHLNS

SEQ ID NO: 20 - protein, 7 aa, Artificial Sequence
  Description of Artificial Sequence Synthetic peptide
SKLYLNN

SEQ ID NO: 21 - protein, 7 aa, Artificial Sequence
  Description of Artificial Sequence Synthetic peptide
DRSNLTR

SEQ ID NO: 22 - protein, 7 aa, Artificial Sequence
  Description of Artificial Sequence Synthetic peptide
YRWLRNS

SEQ ID NO: 23 - protein, 7 aa, Artificial Sequence
  Description of Artificial Sequence Synthetic peptide
HGQTLNE

SEQ ID NO: 24 - protein, 7 aa, Artificial Sequence
  Description of Artificial Sequence Synthetic peptide
SKQYLIK

SEQ ID NO: 25 - protein, 7 aa, Artificial Sequence
  Description of Artificial Sequence Synthetic peptide
DRSHLTR

SEQ ID NO: 26 - protein, 7 aa, Artificial Sequence
  Description of Artificial Sequence Synthetic peptide
DRSHLSR

SEQ ID NO: 27 - protein, 7 aa, Artificial Sequence
  Description of Artificial Sequence Synthetic peptide
TSGNLTR

SEQ ID NO: 28 - protein, 7 aa, Artificial Sequence
  Description of Artificial Sequence Synthetic peptide
LRHNLRA

SEQ ID NO: 29 - protein, 7 aa, Artificial Sequence
  Description of Artificial Sequence Synthetic peptide
DRSHLAR

SEQ ID NO: 30 - protein, 7 aa, Artificial Sequence
  Description of Artificial Sequence Synthetic peptide
RSDHLSE

SEQ ID NO: 31 - DNA, 28 bp, Artificial Sequence
  Description of Artificial Sequence Synthetic oligonucleotide
ctgaaggtggcaatggttcctctctgct

SEQ ID NO: 32 - DNA, 28 bp, Artificial Sequence
  Description of Artificial Sequence Synthetic oligonucleotide
tttcctgtaacgatcgggaactggcatc

SEQ ID NO: 33 - DNA, 28 bp, Artificial Sequence
  Description of Artificial Sequence Synthetic oligonucleotide
gtgcccagggtgctggagaatccaaatg

SEQ ID NO: 34 - DNA, 28 bp, Artificial Sequence
  Description of Artificial Sequence Synthetic oligonucleotide
aggactttgaccatcagaggacatttgg

SEQ ID NO: 35 - DNA, 28 bp, Artificial Sequence
  Description of Artificial Sequence Synthetic oligonucleotide
ctggaggctgccaggaacctacattgtg

SEQ ID NO: 36 - DNA, 28 bp, Artificial Sequence
  Description of Artificial Sequence Synthetic oligonucleotide
gtggtgctgatggaggagacccagaggc

SEQ ID NO: 37 - DNA, 51 bp, Xenopus sp.
tgcttgttctttttgcagaagctcagaataaacgctcaactttggcagatc

SEQ ID NO: 38 - DNA, 284 bp, Homo sapiens
ctagaagctcgctttcttgctgtccaatttctattaaaggttcctttgttccctaagtccaactactaaactgggggatattatgaagggccttgagcatctggattctgcctaataaaaaacatttattttcattgctgcgctagaagctcgctttcttgctgtccaatttctattaaaggttcctttgttccctaagtccaactactaaactgggggatattatgaagggccttgagcatctggattctgcctaataaaaaacatttattttcattgctgcg

SEQ ID NO: 39 - DNA, 592 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 40 - protein, 196 aa, Flavobacterium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 41 - protein, 5 aa, Artificial Sequence
  Description of Artificial Sequence Synthetic peptide
TGEKP

SEQ ID NO: 42 - protein, 6 aa, Artificial Sequence
  Description of Artificial Sequence Synthetic peptide
TGGQRP

SEQ ID NO: 43 - protein, 5 aa, Artificial Sequence
  Description of Artificial Sequence Synthetic peptide
TGQKP

SEQ ID NO: 44 - protein, 6 aa, Artificial Sequence
  Description of Artificial Sequence Synthetic peptide
TGSQKP

SEQ ID NO: 45 - protein, 9 aa, Unknown
  Description of Unknown “LAGLIDADG” family motif peptide
LAGLIDADG

SEQ ID NO: 46 - DNA, 64 bp, Artificial Sequence
  Description of Artificial Sequence Synthetic oligonucleotide
aaaaaaaaaaaaaaaaaaaaaaaaaaaaaaaaaaaaaaaaaaaaaaaaaaaaaaaaaaaaaaaa

SEQ ID NO: 47 - DNA, 128 bp, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - DNA, 193 bp, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa